SEQUENCE LISTING (159 sequences)

SEQ ID NO: 1 - DNA, 20 bp, synthetic construct
  misc_feature 1..20: note = Description of sequence: hATF3-F
  source 1..20: mol_type = other DNA
cctctgcgctggaatcagtc

SEQ ID NO: 2 - DNA, 22 bp, synthetic construct
  misc_feature 1..22: note = Description of sequence: hATF3-R
  source 1..22: mol_type = other DNA
ttctttctcgtcgcctcttttt

SEQ ID NO: 3 - DNA, 20 bp, synthetic construct
  misc_feature 1..20: note = Description of sequence: qPCR-S18-F
  source 1..20: mol_type = other DNA
cttcaacctccctcaccatt

SEQ ID NO: 4 - DNA, 20 bp, synthetic construct
  misc_feature 1..20: note = Description of sequence: qPCR-S18-R
  source 1..20: mol_type = other DNA
tagatagggctcaggcgttt

SEQ ID NO: 5 - DNA, 18 bp, synthetic construct
  misc_feature 1..18: note = Description of sequence: tRNA(Phe)-F
  source 1..18: mol_type = other DNA
gccgaaatagctcagttg

SEQ ID NO: 6 - DNA, 19 bp, synthetic construct
  misc_feature 1..19: note = Description of sequence: tRNA(Phe)-R
  source 1..19: mol_type = other DNA
cagggacctttagatcttc

SEQ ID NO: 7 - DNA, 19 bp, synthetic construct
  misc_feature 1..19: note = Description of sequence: tRNA(Leu)-F
  source 1..19: mol_type = other DNA
gtcaggatggccgagcggt

SEQ ID NO: 8 - DNA, 19 bp, synthetic construct
  misc_feature 1..19: note = Description of sequence: tRNA(Leu)-R
  source 1..19: mol_type = other DNA
tgtcaggagtgggattcga

SEQ ID NO: 9 - DNA, 30 bp, synthetic construct
  misc_feature 1..30: note = Description of sequence: tRNAPhe-probe-1
  source 1..30: mol_type = other DNA
cgaaacccgggatcgaaccagggaccttta

SEQ ID NO: 10 - DNA, 30 bp, synthetic construct
  misc_feature 1..30: note = Description of sequence: tRNAPhe-probe-2
  source 1..30: mol_type = other DNA
ctaacgctctcccaactgagctatttcggc

SEQ ID NO: 11 - DNA, 30 bp, synthetic construct
  misc_feature 1..30: note = Description of sequence: tRNALeu-probe
  source 1..30: mol_type = other DNA
tgtcagaagtgggattcgaacccacgcctc

SEQ ID NO: 12 - DNA, 30 bp, synthetic construct
  misc_feature 1..30: note = Description of sequence: tRNAAla-probe
  source 1..30: mol_type = other DNA
agcacgcgctctaccactgagctacacccc

SEQ ID NO: 13 - DNA, 20 bp, synthetic construct
  misc_feature 1..20: note = Description of sequence: tRNAGly-probe
  source 1..20: mol_type = other DNA
taccactgaaccaccaatgc

SEQ ID NO: 14 - DNA, 20 bp, synthetic construct
  misc_feature 1..20: note = Description of sequence: tRNALys-probe
  source 1..20: mol_type = other DNA
accgactgagctagccgggc

SEQ ID NO: 15 - DNA, 20 bp, synthetic construct
  misc_feature 1..20: note = Description of sequence: tRNASer-probe
  source 1..20: mol_type = other DNA
aaccactcggccacctcgtc

SEQ ID NO: 16 - DNA, 30 bp, synthetic construct
  misc_feature 1..30: note = Description of sequence: tRNAThr-probe
  source 1..30: mol_type = other DNA
ccagtgctctaaccactgagctatggagcc

SEQ ID NO: 17 - DNA, 30 bp, synthetic construct
  misc_feature 1..30: note = Description of sequence: tRNATyr-probe
  source 1..30: mol_type = other DNA
tagtcctctgctctaccagctgaactattg

SEQ ID NO: 18 - RNA, 38 nt, synthetic construct
  misc_feature 1..38: note = Description of sequence: tRNA-Phe-Left-side
  source 1..38: mol_type = other RNA
  modified_base 25: mod_base = gm
gctcagttgggagagcgttagactgaagatctaaggtc

SEQ ID NO: 19 - RNA, 38 nt, synthetic construct; identical to SEQ ID NO: 18
  misc_feature 1..38: note = Description of sequence: Phe-Left-No-O
  source 1..38: mol_type = other RNA

SEQ ID NO: 20 - RNA, 38 nt, synthetic construct
  misc_feature 1..38: note = Description of sequence: Phe-anticodon-mut
  source 1..38: mol_type = other RNA
  modified_base 25: mod_base = gm
gctcagttgggagagcgttagactgttgatctaaggtc

SEQ ID NO: 21 - RNA, 34 nt, synthetic construct
  misc_feature 1..34: note = Description of sequence: Phe-Left-34
  source 1..34: mol_type = other RNA
  modified_base 25: mod_base = gm
gctcagttgggagagcgttagactgaagatctaa

SEQ ID NO: 22 - RNA, 34 nt, synthetic construct
  misc_feature 1..34: note = Description of sequence: coli-Phe-left-34
  source 1..34: mol_type = other RNA
  modified_base 25: mod_base = gm
gctcagtcggtagagcagggcattgaaaatgccc

SEQ ID NO: 23 - RNA, 38 nt, synthetic construct; identical to SEQ ID NO: 18
  misc_feature 1..38: note = Description of sequence: Phe-half-DNA
  source 1..38: mol_type = other RNA
  modified_base 25: mod_base = gm

SEQ ID NO: 24 - RNA, 17 nt, synthetic construct
  misc_feature 1..17: note = Description of sequence: Phe-17-No-O
  source 1..17: mol_type = other RNA
ttagactgaagatctaa

SEQ ID NO: 25 - RNA, 14 nt, synthetic construct
  misc_feature 1..14: note = Description of sequence: Phe-17-mut1
  source 1..14: mol_type = other RNA
  modified_base 8: mod_base = gm
ttagagagttctaa

SEQ ID NO: 26 - RNA, 17 nt, synthetic construct
  misc_feature 1..17: note = Description of sequence: Phe-17-mut2
  source 1..17: mol_type = other RNA
  modified_base 8: mod_base = gm
ggctgctgaagacagcc

SEQ ID NO: 27 - RNA, 17 nt, synthetic construct
  misc_feature 1..17: note = Description of sequence: Phe-17-mut3
  source 1..17: mol_type = other RNA
  modified_base 6: mod_base = cm
ttagactgaagatctaa

SEQ ID NO: 28 - DNA, 17 bp, synthetic construct
  misc_feature 1..17: note = Description of sequence: Phe-17-DNA Phe-17-DNA#2 and Phe-17-DNA#3
  source 1..17: mol_type = other DNA
  modified_base 8: mod_base = gm
ttagactgaagatctaa

SEQ ID NO: 29 - DNA, 17 bp, synthetic construct
  misc_feature 1..17: note = Description of sequence: Phe-17-DNA#4 and Phe-17-DNA#5
  source 1..17: mol_type = other DNA
  modified_base 8: mod_base = gm
ttagactgaagatctaa

SEQ ID NO: 30 - DNA, 17 bp, synthetic construct
  misc_feature 1..17: note = Description of sequence: Phe-17-DNA#6
  source 1..17: mol_type = other DNA
  modified_base 8: mod_base = gm
ttagactgaagatctaa

SEQ ID NO: 31 - RNA, 17 nt, synthetic construct
  misc_feature 1..17: note = Description of sequence: Phe-17-WT and Phe-17-DNA#8
  source 1..17: mol_type = other RNA
  modified_base 8: mod_base = gm
ttagactgaagatctaa

SEQ ID NO: 32 - RNA, 17 nt, synthetic construct
  misc_feature 1..17: note = Description of sequence: Phe-17-mut4
  source 1..17: mol_type = other RNA
  modified_base 8: mod_base = cm
ttagactcaagatctaa

SEQ ID NO: 33 - RNA, 17 nt, synthetic construct
  misc_feature 1..17: note = Description of sequence: Phe-17-mut5
  source 1..17: mol_type = other RNA
  modified_base 8: mod_base = OTHER; note = 2'O-methyl-adenosine
ttagactaaagatctaa

SEQ ID NO: 34 - RNA, 17 nt, synthetic construct
  misc_feature 1..17: note = Description of sequence: Phe-17-mut6
  source 1..17: mol_type = other RNA
  modified_base 8: mod_base = um
ttagacttaagatctaa

SEQ ID NO: 35 - RNA, 17 nt, synthetic construct
  misc_feature 1..17: note = Description of sequence: Phe-17-mut7
  source 1..17: mol_type = other RNA
  modified_base 8: mod_base = gm
ggctgctgaagatctaa

SEQ ID NO: 36 - RNA, 17 nt, synthetic construct
  misc_feature 1..17: note = Description of sequence: Phe-17-mut8
  source 1..17: mol_type = other RNA
  modified_base 8: mod_base = gm
ttagaatgaagatctaa

SEQ ID NO: 37 - RNA, 17 nt, synthetic construct
  misc_feature 1..17: note = Description of sequence: Phe-17-mut9
  source 1..17: mol_type = other RNA
  modified_base 8: mod_base = gm
ttagattgaaggtctaa

SEQ ID NO: 38 - RNA, 17 nt, synthetic construct
  misc_feature 1..17: note = Description of sequence: Phe-17-mut10
  source 1..17: mol_type = other RNA
  modified_base 8: mod_base = gm
ttagagtgaagatctaa

SEQ ID NO: 39 - RNA, 17 nt, synthetic construct
  misc_feature 1..17: note = Description of sequence: Phe-17-mut11
  source 1..17: mol_type = other RNA
  modified_base 8: mod_base = gm
ttagacagaagatctaa

SEQ ID NO: 40 - RNA, 17 nt, synthetic construct
  misc_feature 1..17: note = Description of sequence: Phe-17-mut12
  source 1..17: mol_type = other RNA
  modified_base 8: mod_base = gm
ttagaccgaagatctaa

SEQ ID NO: 41 - RNA, 17 nt, synthetic construct
  misc_feature 1..17: note = Description of sequence: Phe-17-mut13
  source 1..17: mol_type = other RNA
  modified_base 8: mod_base = gm
ttagacggaagatctaa

SEQ ID NO: 42 - RNA, 17 nt, synthetic construct
  misc_feature 1..17: note = Description of sequence: Phe-17-DNA#7
  source 1..17: mol_type = other RNA
  modified_base 8: mod_base = gm
ttagactgaagatctaa

SEQ ID NO: 43 - RNA, 17 nt, synthetic construct
  misc_feature 1..17: note = Description of sequence: Phe-17-mut13
  source 1..17: mol_type = other RNA
  modified_base 9: mod_base = gm
ttagaactgaagtctaa

SEQ ID NO: 44 - RNA, 17 nt, synthetic construct
  misc_feature 1..17: note = Description of sequence: Phe-17-mut14
  source 1..17: mol_type = other RNA
  modified_base 10: mod_base = gm
ttagagactgaatctaa

SEQ ID NO: 45 - RNA, 17 nt, synthetic construct
  misc_feature 1..17: note = Description of sequence: Phe-17-mut15
  source 1..17: mol_type = other RNA
  modified_base 11: mod_base = gm
ttagaagactgatctaa

SEQ ID NO: 46 - RNA, 14 nt, synthetic construct
  misc_feature 1..14: note = Description of sequence: Phe-17-m7-com1
  source 1..14: mol_type = other RNA
ttagatcttcagcc

SEQ ID NO: 47 - RNA, 17 nt, synthetic construct
  misc_feature 1..17: note = Description of sequence: Phe-17-m7-com2
  source 1..17: mol_type = other RNA
ttagatagaacgcagcc

SEQ ID NO: 48 - RNA, 15 nt, synthetic construct
  misc_feature 1..15: note = Description of sequence: Phe-17-mut16
  source 1..15: mol_type = other RNA
  modified_base 7: mod_base = gm
tagactgaagatcta

SEQ ID NO: 49 - RNA, 13 nt, synthetic construct
  misc_feature 1..13: note = Description of sequence: Phe-17-mut17
  source 1..13: mol_type = other RNA
  modified_base 6: mod_base = gm
agactgaagatct

SEQ ID NO: 50 - RNA, 11 nt, synthetic construct
  misc_feature 1..11: note = Description of sequence: Phe-17-mut18
  source 1..11: mol_type = other RNA
  modified_base 5: mod_base = gm
gactgaagatc

SEQ ID NO: 51 - RNA, 16 nt, synthetic construct
  misc_feature 1..16: note = Description of sequence: Phe-17-mut19
  source 1..16: mol_type = other RNA
  modified_base 8: mod_base = gm
ttagactgagatctaa

SEQ ID NO: 52 - RNA, 15 nt, synthetic construct
  misc_feature 1..15: note = Description of sequence: Phe-17-mut20
  source 1..15: mol_type = other RNA
  modified_base 8: mod_base = gm
ttagactggatctaa

SEQ ID NO: 53 - RNA, 14 nt, synthetic construct
  misc_feature 1..14: note = Description of sequence: Phe-17-mut21
  source 1..14: mol_type = other RNA
  modified_base 8: mod_base = gm
ttagactgatctaa

SEQ ID NO: 54 - RNA, 13 nt, synthetic construct
  misc_feature 1..13: note = Description of sequence: Phe-17-mut22
  source 1..13: mol_type = other RNA
  modified_base 8: mod_base = gm
ttagattgtctaa

SEQ ID NO: 55 - DNA, 20 bp, synthetic construct
  misc_feature 1..20: note = Description of sequence: FTJS1 guide
  source 1..20: mol_type = other DNA
atctgtaccacacctggtag

SEQ ID NO: 56 - DNA, 21 bp, synthetic construct
  misc_feature 1..21: note = Description of sequence: tRNAPhe (GAA)
  source 1..21: mol_type = other DNA
tcagttgggagagcgttagac

SEQ ID NO: 57 - DNA, 21 bp, synthetic construct
  misc_feature 1..21: note = Description of sequence: tRNALeu (CAA)
  source 1..21: mol_type = other DNA
tctaaggcgccagactcaagt

SEQ ID NO: 58 - DNA, 61 bp, synthetic construct
  misc_feature 1..61: note = Description of sequence: Ligation adaptor
  source 1..61: mol_type = other DNA
aatgatacggcgaccaccgagatctacactctttccctacacgacgctcttccgatctggg

SEQ ID NO: 59 - DNA, 81 bp, synthetic construct
  misc_feature 1..81: note = Description of sequence: RT Primer
  source 1..81: mol_type = other DNA
  variation 81: note = n is a, c, g, or t
gcatctcggtggtccaatctgaactccagtcgtgtgactggagttcagacgtgtgctcttccgatctttttttttttttvn

SEQ ID NO: 60 - DNA, 50 bp, synthetic construct
  misc_feature 1..50: note = Description of sequence: Forward primer
  source 1..50: mol_type = other DNA
aatgatacggcgaccaccgagatctacactctttccctacacgacgctct

SEQ ID NO: 61 - DNA, 58 bp, synthetic construct
  misc_feature 1..58: note = Description of sequence: Library-PCR-R-13
  source 1..58: mol_type = other DNA
caagcagaagacggcatacgagattgttgactgtgactggagttcagacgtgtgctct

SEQ ID NO: 62 - DNA, 58 bp, synthetic construct
  misc_feature 1..58: note = Description of sequence: Library-PCR-R-14
  source 1..58: mol_type = other DNA
caagcagaagacggcatacgagatacggaactgtgactggagttcagacgtgtgctct

SEQ ID NO: 63 - DNA, 58 bp, synthetic construct
  misc_feature 1..58: note = Description of sequence: Library-PCR-R-15
  source 1..58: mol_type = other DNA
caagcagaagacggcatacgagattctgacatgtgactggagttcagacgtgtgctct

SEQ ID NO: 64 - DNA, 58 bp, synthetic construct
  misc_feature 1..58: note = Description of sequence: Library-PCR-R-16
  source 1..58: mol_type = other DNA
caagcagaagacggcatacgagatcgggacgggtgactggagttcagacgtgtgctct

SEQ ID NO: 65 - DNA, 58 bp, synthetic construct
  misc_feature 1..58: note = Description of sequence: Library-PCR-R-18
  source 1..58: mol_type = other DNA
caagcagaagacggcatacgagatgtgcggacgtgactggagttcagacgtgtgctct

SEQ ID NO: 66 - DNA, 58 bp, synthetic construct
  misc_feature 1..58: note = Description of sequence: Library-PCR-R-19
  source 1..58: mol_type = other DNA
caagcagaagacggcatacgagatcgtttcacgtgactggagttcagacgtgtgctct

SEQ ID NO: 67 - DNA, 58 bp, synthetic construct
  misc_feature 1..58: note = Description of sequence: Library-PCR-R-20
  source 1..58: mol_type = other DNA
caagcagaagacggcatacgagataaggccacgtgactggagttcagacgtgtgctct

SEQ ID NO: 68 - DNA, 58 bp, synthetic construct
  misc_feature 1..58: note = Description of sequence: Library-PCR-R-21
  source 1..58: mol_type = other DNA
caagcagaagacggcatacgagattccgaaacgtgactggagttcagacgtgtgctct

SEQ ID NO: 69 - DNA, 34 bp, synthetic construct
  misc_feature 1..34: note = Description of sequence: tRNALeu-Forward
  source 1..34: mol_type = other DNA
atgcgaattccagacactggagagccgacccagc

SEQ ID NO: 70 - DNA, 34 bp, synthetic construct
  misc_feature 1..34: note = Description of sequence: tRNALeu-Reverse
  source 1..34: mol_type = other DNA
atgcctgcagcccggtacctaaaagccgattctt

SEQ ID NO: 71 - DNA, 62 bp, synthetic construct
  misc_feature 1..62: note = Description of sequence: tRNAPhe-Forward
  source 1..62: mol_type = other DNA
atctaaaggtccctggttcgatcccgggtttcggcacatacttttctttttcctctccttac

SEQ ID NO: 72 - DNA, 60 bp, synthetic construct
  misc_feature 1..60: note = Description of sequence: tRNAPhe-Reverse
  source 1..60: mol_type = other DNA
cttcagtctaacgctctcccaactgagctatttcggcacactgcataacagccctgattt

SEQ ID NO: 73 - DNA, 25 bp, synthetic construct
  misc_feature 1..25: note = Description of sequence: CRISPR-FTSJ1#1S
  source 1..25: mol_type = other DNA
caccgatctgtaccacacctggtag

SEQ ID NO: 74 - DNA, 25 bp, synthetic construct
  misc_feature 1..25: note = Description of sequence: CRISPR-FTSJ1#1C
  source 1..25: mol_type = other DNA
aaacctaccaggtgtggtacagatc

SEQ ID NO: 75 - DNA, 32 bp, synthetic construct
  misc_feature 1..32: note = Description of sequence: FTSJ1-NheI
  source 1..32: mol_type = other DNA
ctatcagctagcatgggacggacgtcaaagga

SEQ ID NO: 76 - DNA, 30 bp, synthetic construct
  misc_feature 1..30: note = Description of sequence: FTSJ1-BamHI
  source 1..30: mol_type = other DNA
taatggatccttaaggtgaacaactcattt

SEQ ID NO: 77 - DNA, 45 bp, synthetic construct
  misc_feature 1..45: note = Description of sequence: EcoRI-FTSJ1-A26P
  source 1..45: mol_type = other DNA
tggaattccttatccagttgtagaagcttgaaggggctgcgagca

SEQ ID NO: 78 - DNA, 43 bp, synthetic construct
  misc_feature 1..43: note = Description of sequence: EcoRI-FTSJ1-K28A
  source 1..43: mol_type = other DNA
tggaattccttatccaattgtagcagtgcgaaggcgctgcgag

SEQ ID NO: 79 - DNA, 40 bp, synthetic construct
  misc_feature 1..40: note = Description of sequence: 5 -SacII-hSAMD9
  source 1..40: mol_type = other DNA
ctccgcggccgcccccttcaccatggcaaagcaacttaac

SEQ ID NO: 80 - DNA, 25 bp, synthetic construct
  misc_feature 1..25: note = Description of sequence: hSAMD9-Hind3
  source 1..25: mol_type = other DNA
ggctttctttgtaagctttgaccac

SEQ ID NO: 81 - DNA, 32 bp, synthetic construct
  misc_feature 1..32: note = Description of sequence: h9-E184A-F
  source 1..32: mol_type = other DNA
agtctacagccggcaacaggaccaggcaatct

SEQ ID NO: 82 - DNA, 32 bp, synthetic construct
  misc_feature 1..32: note = Description of sequence: h9-E184A-R
  source 1..32: mol_type = other DNA
ctggtcctgttgccggctgtagactaaaatcc

SEQ ID NO: 83 - DNA, 35 bp, synthetic construct
  misc_feature 1..35: note = Description of sequence: h9-D192A-F
  source 1..35: mol_type = other DNA
atctcattgctccgattcatgaattcaaagccttc

SEQ ID NO: 84 - DNA, 35 bp, synthetic construct
  misc_feature 1..35: note = Description of sequence: h9-D192A-R
  source 1..35: mol_type = other DNA
gaattcatgaatcggagcaatgagattgcctggtc

SEQ ID NO: 85 - DNA, 31 bp, synthetic construct
  misc_feature 1..31: note = Description of sequence: h9-E196A-F
  source 1..31: mol_type = other DNA
atccgatacatgcattcaaagccttcacaaa

SEQ ID NO: 86 - DNA, 30 bp, synthetic construct
  misc_feature 1..30: note = Description of sequence: h9-E196A-R
  source 1..30: mol_type = other DNA
aggctttgaatgcatgtatcggatcaatga

SEQ ID NO: 87 - DNA, 34 bp, synthetic construct
  misc_feature 1..34: note = Description of sequence: hD9-E218A-F
  source 1..34: mol_type = other DNA
gaaatttagcaatgcggttttccgatttgcttca

SEQ ID NO: 88 - DNA, 34 bp, synthetic construct
  misc_feature 1..34: note = Description of sequence: hD9-E218A-R
  source 1..34: mol_type = other DNA
caaatcggaaaaccgcattgctaaatttcatctt

SEQ ID NO: 89 - DNA, 34 bp, synthetic construct
  misc_feature 1..34: note = Description of sequence: hD9-D241A-F
  source 1..34: mol_type = other DNA
ttttggagtcaaagcgaaaccccatgggaaaatt

SEQ ID NO: 90 - DNA, 34 bp, synthetic construct
  misc_feature 1..34: note = Description of sequence: hD9-D241A-R
  source 1..34: mol_type = other DNA
tcccatggggtttcgctttgactccaaaatgaat

SEQ ID NO: 91 - DNA, 34 bp, synthetic construct
  misc_feature 1..34: note = Description of sequence: hD9-D345A-F
  source 1..34: mol_type = other DNA
actatttgtgcgagcggggaccagctctaaggac

SEQ ID NO: 92 - DNA, 34 bp, synthetic construct
  misc_feature 1..34: note = Description of sequence: hD9-D345A-R
  source 1..34: mol_type = other DNA
tagagctggtccccgctcgcacaaatagtgagaa

SEQ ID NO: 93 - DNA, 22 bp, synthetic construct
  misc_feature 1..22: note = Description of sequence: qATF3-F2
  source 1..22: mol_type = other DNA
agaggcgacgagaaagaaataa

SEQ ID NO: 94 - DNA, 20 bp, synthetic construct
  misc_feature 1..20: note = Description of sequence: qATF3-R2
  source 1..20: mol_type = other DNA
tcacactttccagcttctcc

SEQ ID NO: 95 - DNA, 22 bp, synthetic construct
  misc_feature 1..22: note = Description of sequence: qActb-F
  source 1..22: mol_type = other DNA
caccattggcaatgagcggttc

SEQ ID NO: 96 - DNA, 22 bp, synthetic construct
  misc_feature 1..22: note = Description of sequence: qActb-R
  source 1..22: mol_type = other DNA
aggtctttgcggatgtccacgt

SEQ ID NO: 97 - DNA, 20 bp, synthetic construct
  misc_feature 1..20: note = Description of sequence: hATF3-F
  source 1..20: mol_type = other DNA
cctctgcgctggaatcagtc

SEQ ID NO: 98 - DNA, 22 bp, synthetic construct
  misc_feature 1..22: note = Description of sequence: hATF3-R
  source 1..22: mol_type = other DNA
ttctttctcgtcgcctcttttt

SEQ ID NO: 99 - DNA, 20 bp, synthetic construct
  misc_feature 1..20: note = Description of sequence: qPCR-S18-F
  source 1..20: mol_type = other DNA
cttcaacctccctcaccatt

SEQ ID NO: 100 - DNA, 20 bp, synthetic construct
  misc_feature 1..20: note = Description of sequence: qPCR-S18-R
  source 1..20: mol_type = other DNA
tagatagggctcaggcgttt

SEQ ID NO: 101 - DNA, 18 bp, synthetic construct
  misc_feature 1..18: note = Description of sequence: tRNA(Phe)-F
  source 1..18: mol_type = other DNA
gccgaaatagctcagttg

SEQ ID NO: 102 - DNA, 19 bp, synthetic construct
  misc_feature 1..19: note = Description of sequence: tRNA(Phe)-R
  source 1..19: mol_type = other DNA
cagggacctttagatcttc

SEQ ID NO: 103 - DNA, 19 bp, synthetic construct
  misc_feature 1..19: note = Description of sequence: tRNA(Leu)-F
  source 1..19: mol_type = other DNA
gtcaggatggccgagcggt

SEQ ID NO: 104 - DNA, 19 bp, synthetic construct
  misc_feature 1..19: note = Description of sequence: tRNA(Leu)-R
  source 1..19: mol_type = other DNA
tgtcaggagtgggattcga

SEQ ID NO: 105 - DNA, 30 bp, synthetic construct; identical to SEQ ID NO: 9
  misc_feature 1..30: note = Description of sequence: tRNAPhe-probe-1
  source 1..30: mol_type = other DNA

SEQ ID NO: 106 - DNA, 30 bp, synthetic construct; identical to SEQ ID NO: 10
  misc_feature 1..30: note = Description of sequence: tRNAPhe-probe-2
  source 1..30: mol_type = other DNA

SEQ ID NO: 107 - DNA, 30 bp, synthetic construct; identical to SEQ ID NO: 11
  misc_feature 1..30: note = Description of sequence: TRNALeu-probe
  source 1..30: mol_type = other DNA

SEQ ID NO: 108 - DNA, 30 bp, synthetic construct; identical to SEQ ID NO: 12
  misc_feature 1..30: note = Description of sequence: tRNAAla-probe
  source 1..30: mol_type = other DNA

SEQ ID NO: 109 - DNA, 20 bp, synthetic construct
  misc_feature 1..20: note = Description of sequence: tRNAGly-probe
  source 1..20: mol_type = other DNA
taccactgaaccaccaatgc

SEQ ID NO: 110 - DNA, 20 bp, synthetic construct
  misc_feature 1..20: note = Description of sequence: tRNALys-probe
  source 1..20: mol_type = other DNA
accgactgagctagccgggc

SEQ ID NO: 111 - DNA, 20 bp, synthetic construct
  misc_feature 1..20: note = Description of sequence: tRNASer-probe
  source 1..20: mol_type = other DNA
aaccactcggccacctcgtc

SEQ ID NO: 112 - DNA, 30 bp, synthetic construct; identical to SEQ ID NO: 16
  misc_feature 1..30: note = Description of sequence: tRNAThr-probe
  source 1..30: mol_type = other DNA

SEQ ID NO: 113 - DNA, 30 bp, synthetic construct; identical to SEQ ID NO: 17
  misc_feature 1..30: note = Description of sequence: tRNATyr-probe
  source 1..30: mol_type = other DNA

SEQ ID NO: 114 - RNA, 38 nt, synthetic construct; identical to SEQ ID NO: 18
  misc_feature 1..38: note = Description of sequence: 38WT
  source 1..38: mol_type = other RNA
  modified_base 25: mod_base = gm

SEQ ID NO: 115 - RNA, 38 nt, synthetic construct; identical to SEQ ID NO: 18
  misc_feature 1..38: note = Description of sequence: 38m-
  source 1..38: mol_type = other RNA

SEQ ID NO: 116 - RNA, 38 nt, synthetic construct; identical to SEQ ID NO: 20
  misc_feature 1..38: note = Description of sequence: 38-mut1
  source 1..38: mol_type = other RNA
  modified_base 25: mod_base = gm

SEQ ID NO: 117 - RNA, 34 nt, synthetic construct; identical to SEQ ID NO: 21
  misc_feature 1..34: note = Description of sequence: 34WT
  source 1..34: mol_type = other RNA
  modified_base 25: mod_base = gm

SEQ ID NO: 118 - RNA, 34 nt, synthetic construct; identical to SEQ ID NO: 22
  misc_feature 1..34: note = Description of sequence: 34-mut1
  source 1..34: mol_type = other RNA
  modified_base 25: mod_base = gm

SEQ ID NO: 119 - RNA, 17 nt, synthetic construct
  misc_feature 1..17: note = Description of sequence: 17WT
  source 1..17: mol_type = other RNA
  modified_base 8: mod_base = gm
ttagactgaagatctaa

SEQ ID NO: 120 - RNA, 17 nt, synthetic construct
  misc_feature 1..17: note = Description of sequence: 17m-
  source 1..17: mol_type = other RNA
ttagactgaagatctaa

SEQ ID NO: 121 - RNA, 17 nt, synthetic construct
  misc_feature 1..17: note = Description of sequence: mut1
  source 1..17: mol_type = other RNA
  modified_base 8: mod_base = gm
ttagagagttcttctaa

SEQ ID NO: 122 - RNA, 17 nt, synthetic construct
  misc_feature 1..17: note = Description of sequence: mut2
  source 1..17: mol_type = other RNA
  modified_base 8: mod_base = gm
ggctgctgaagacagcc

SEQ ID NO: 123 - RNA, 17 nt, synthetic construct
  misc_feature 1..17: note = Description of sequence: mut3
  source 1..17: mol_type = other RNA
  modified_base 6: mod_base = cm
ttagactgaagatctaa

SEQ ID NO: 124 - RNA, 17 nt, synthetic construct
  misc_feature 1..17: note = Description of sequence: mut4
  source 1..17: mol_type = other RNA
  modified_base 8: mod_base = cm
ttagactcaagatctaa

SEQ ID NO: 125 - RNA, 17 nt, synthetic construct
  misc_feature 1..17: note = Description of sequence: mut5
  source 1..17: mol_type = other RNA
  modified_base 8: mod_base = OTHER; note = 2'O-methyl-adenosine
ttagactaaagatctaa

SEQ ID NO: 126 - RNA, 17 nt, synthetic construct
  misc_feature 1..17: note = Description of sequence: mut6
  source 1..17: mol_type = other RNA
  modified_base 8: mod_base = um
ttagacttaagatctaa

SEQ ID NO: 127 - RNA, 17 nt, synthetic construct
  misc_feature 1..17: note = Description of sequence: mut7
  source 1..17: mol_type = other RNA
  modified_base 8: mod_base = gm
ggctgctgaagatctaa

SEQ ID NO: 128 - RNA, 17 nt, synthetic construct
  misc_feature 1..17: note = Description of sequence: mut8
  source 1..17: mol_type = other RNA
  modified_base 8: mod_base = gm
ttagaatgaagatctaa

SEQ ID NO: 129 - RNA, 17 nt, synthetic construct
  misc_feature 1..17: note = Description of sequence: mut9
  source 1..17: mol_type = other RNA
  modified_base 8: mod_base = gm
ttagattgaaggtctaa

SEQ ID NO: 130 - RNA, 17 nt, synthetic construct
  misc_feature 1..17: note = Description of sequence: mut10
  source 1..17: mol_type = other RNA
  modified_base 8: mod_base = gm
ttagagtgaagatctaa

SEQ ID NO: 131 - RNA, 17 nt, synthetic construct
  misc_feature 1..17: note = Description of sequence: mut11
  source 1..17: mol_type = other RNA
  modified_base 8: mod_base = gm
ttagacagaagatctaa

SEQ ID NO: 132 - RNA, 17 nt, synthetic construct
  misc_feature 1..17: note = Description of sequence: mut12
  source 1..17: mol_type = other RNA
  modified_base 8: mod_base = gm
ttagaccgaagatctaa

SEQ ID NO: 133 - RNA, 17 nt, synthetic construct
  misc_feature 1..17: note = Description of sequence: mut13A
  source 1..17: mol_type = other RNA
  modified_base 9: mod_base = gm
ttagaactgaagtctaa

SEQ ID NO: 134 - RNA, 17 nt, synthetic construct
  misc_feature 1..17: note = Description of sequence: mut13B
  source 1..17: mol_type = other RNA
  modified_base 8: mod_base = gm
ttagacggaagatctaa

SEQ ID NO: 135 - RNA, 17 nt, synthetic construct
  misc_feature 1..17: note = Description of sequence: mut14
  source 1..17: mol_type = other RNA
  modified_base 10: mod_base = gm
ttagagactgaatctaa

SEQ ID NO: 136 - RNA, 17 nt, synthetic construct
  misc_feature 1..17: note = Description of sequence: mut15
  source 1..17: mol_type = other RNA
  modified_base 11: mod_base = gm
ttagaagactgatctaa

SEQ ID NO: 137 - RNA, 15 nt, synthetic construct
  misc_feature 1..15: note = Description of sequence: mut16
  source 1..15: mol_type = other RNA
  modified_base 7: mod_base = gm
tagactgaagatcta

SEQ ID NO: 138 - RNA, 13 nt, synthetic construct
  misc_feature 1..13: note = Description of sequence: mut17
  source 1..13: mol_type = other RNA
  modified_base 6: mod_base = gm
agactgaagatct

SEQ ID NO: 139 - RNA, 11 nt, synthetic construct
  misc_feature 1..11: note = Description of sequence: mut18
  source 1..11: mol_type = other RNA
  modified_base 5: mod_base = gm
gactgaagatc

SEQ ID NO: 140 - RNA, 16 nt, synthetic construct
  misc_feature 1..16: note = Description of sequence: mut19
  source 1..16: mol_type = other RNA
  modified_base 8: mod_base = gm
ttagactgagatctaa

SEQ ID NO: 141 - RNA, 15 nt, synthetic construct
  misc_feature 1..15: note = Description of sequence: mut20
  source 1..15: mol_type = other RNA
  modified_base 8: mod_base = gm
ttagactggatctaa

SEQ ID NO: 142 - RNA, 14 nt, synthetic construct
  misc_feature 1..14: note = Description of sequence: mut21
  source 1..14: mol_type = other RNA
  modified_base 8: mod_base = gm
ttagactgatctaa

SEQ ID NO: 143 - RNA, 13 nt, synthetic construct
  misc_feature 1..13: note = Description of sequence: mut22
  source 1..13: mol_type = other RNA
  modified_base 8: mod_base = gm
ttagattgtctaa

SEQ ID NO: 144 - RNA, 18 nt, synthetic construct
  misc_feature 1..18: note = Description of sequence: mut23
  source 1..18: mol_type = other RNA
  modified_base 8: mod_base = gm
ttagactgatagatctaa

SEQ ID NO: 145 - RNA, 19 nt, synthetic construct
  misc_feature 1..19: note = Description of sequence: mut24
  source 1..19: mol_type = other RNA
  modified_base 8: mod_base = gm
ttagactgatgagatctaa

SEQ ID NO: 146 - DNA, 38 bp, synthetic construct
  misc_feature 1..38: note = Description of sequence: 38DNA
  source 1..38: mol_type = other DNA
  modified_base 25: mod_base = gm
gctcagttgggagagcgttagactgaagatctaaggtc

SEQ ID NO: 147 - DNA, 17 bp, synthetic construct
  misc_feature 1..17: note = Description of sequence: 17DNA DNA#2 and DNA #3
  source 1..17: mol_type = other DNA
  modified_base 8: mod_base = gm
ttagactgaagatctaa

SEQ ID NO: 148 - DNA, 17 bp, synthetic construct
  misc_feature 1..17: note = Description of sequence: DNA#4 and DNA#5
  source 1..17: mol_type = other DNA
  modified_base 8: mod_base = gm
ttagactgaagatctaa

SEQ ID NO: 149 - DNA, 17 bp, synthetic construct
  misc_feature 1..17: note = Description of sequence: DNA#6
  source 1..17: mol_type = other DNA
  modified_base 8: mod_base = gm
ttagactgaagatctaa

SEQ ID NO: 150 - DNA, 17 bp, synthetic construct
  misc_feature 1..17: note = Description of sequence: DNA#7
  source 1..17: mol_type = other DNA
  modified_base 8: mod_base = gm
ttagactgaagatctaa

SEQ ID NO: 151 - RNA, 17 nt, synthetic construct
  misc_feature 1..17: note = Description of sequence: DNA#8
  source 1..17: mol_type = other RNA
  modified_base 8: mod_base = gm
ttagactgaagatctaa

SEQ ID NO: 152 - skipped

SEQ ID NO: 153 - RNA, 78 nt, synthetic construct
  misc_feature 1..78: note = Description of sequence: yeast tRNAPhe
  source 1..78: mol_type = other RNA
  modified_base 32: mod_base = cm
  modified_base 35: mod_base = gm
gcggatttagctcagttgggagagcgccagactgaayatctggaggtcctgtgtgttcgatccacagaattcgcacca

SEQ ID NO: 154 - RNA, 76 nt, synthetic construct
  misc_feature 1..76: note = Description of sequence: human tRNAPhe
  source 1..76: mol_type = other RNA
  modified_base 32: mod_base = cm
  modified_base 35: mod_base = gm
gccgaaatagctcagttgggagagcgttagactgaayatctaaaggtccctggttcgatcccgggtttcggcacca

SEQ ID NO: 155 - RNA, 76 nt, Homo sapiens
  misc_feature 1..76: note = Description of sequence: human tRNAPhe
  source 1..76: mol_type = other RNA
  modified_base 32: mod_base = cm
  modified_base 34: mod_base = gm
gccgaaatagctcagttgggagagcgttagactgaagatctaaaggtccctggttcgatcccgggtttcggcacca

SEQ ID NO: 156 - RNA, 18 nt, synthetic construct
  misc_feature 1..18: note = Description of sequence: Phe17-Fret
  source 1..18: mol_type = other RNA
  modified_base <1: mod_base = OTHER; note = 6-Carboxyfluorescein
  modified_base 9: mod_base = gm
  modified_base >18: mod_base = OTHER; note = Iowa Black fluorescent quencher
ttagactggaagatctaa

SEQ ID NO: 157 - RNA, 18 nt, synthetic construct
  misc_feature 1..18: note = Description of sequence: Phe17-Fret-noM
  source 1..18: mol_type = other RNA
  modified_base <1: mod_base = OTHER; note = 6-Carboxyfluorescein
  modified_base >18: mod_base = OTHER; note = Iowa Black fluorescent quencher
ttagactggaagatctaa

SEQ ID NO: 158 - RNA, 12 nt, synthetic construct
  misc_feature 1..12: note = Description of sequence: Phe17-no6-Fam
  source 1..12: mol_type = other RNA
  modified_base 3: mod_base = gm
  modified_base >12: mod_base = OTHER; note = Iowa Black fluorescent quencher
ctgaagatctaa

SEQ ID NO: 159 - skipped

(19) United States
(12) Patent Application Publication
(10) Pub. No.: US 2026/0242789 A1
(43) Pub. Date: Aug. 20, 2026

(54) METHODS OF TARGETING AND TREATING SAMD9 OR SAMD9L RELATED DISEASES

(71) Applicants: THE BOARD OF REGENTS FOR THE OKLAHOMA AGRICULTURAL AND MECHANICAL COLLEGES, Stillwater, OK (US); CORNELL UNIVERSITY, Ithaca, NY (US); BOARD OF REGENTS, THE UNIVERSITY OF TEXAS SYSTEM, Austin, TX (US)

(72) Inventors: Yan XIANG, Austin, TX (US); Fushun ZHANG, Austin, TX (US); Junpeng DENG, Stillwater, OK (US); Shu-bing QIAN, Ithaca, NY (US)

(21) Appl. No.: 19/161,457

(22) PCT Filed: Mar. 1, 2024
(86) PCT No.: PCT/US2024/017983

§ 371 Date: Sep. 2, 2025

Related U.S. Application Data
(60) Provisional application No. 63/487,939, filed on Mar. 2, 2023.

(51) Int. Cl. C12N 15/113; A61P 31/12; A61P 35/00

(52) U.S. Cl. CPC C12N 15/113; A61P 31/12; A61P 35/00

(57) ABSTRACT

The present disclosure relates to compositions to treat diseases caused by SAMD9 and/or SAMD9L mutations, and methods for uses thereof.

### CROSS-REFERENCE TO RELATED APPLICATIONS

This PCT application claims priority to, and the benefit of, U.S. Provisional Patent Application No. 63/487,939, filed Mar. 2, 2023, which is incorporated by reference herein in its entirety.

### STATEMENT REGARDING FEDERALLY SPONSORED RESEARCH

This was made with Government Support under Grant No. AI151638 awarded by the National Institutes of Health. The Government has certain rights in the invention.

### REFERENCE TO SEQUENCE LISTING

The sequence listing submitted on Mar. 1, 2024, as an .XML file entitled “11024-014WO1_ST.26” created on Feb. 29, 2024, and having a file size of 235,431 bytes is hereby incorporated by reference pursuant to 37 C.F.R. § 1.52(e)(5).

### FIELD

The present disclosure relates to compositions to treat diseases caused by SAMD9 and/or SAMD9L mutations, and methods for uses thereof.

### BACKGROUND

Transfer RNA (tRNA) is essential for protein synthesis and are subjected to modification and regulation. tRNA molecules are heavily modified post-transcriptionally to ensure translational accuracy and cell survival. The physiological function of the tRNA modification is not entirely understood, however it is targeted by microbial toxins. tRNAs are a major target for protein toxins employed by microbes to impede virus infection, alleviate cellular stress, or eliminate competing organisms. Many tRNA-targeting protein toxins are anticodon nucleases (ACNase) that cleave at the Anticodon Stem Loops (ASL), leading to depletion of specific tRNAs from cellular pools and a block in protein synthesis. Notable examples of ACNases include PrrC, which is activated upon 14 phage infection and serves as a bacterial innate defense against phages, and Colicin E5, which is secreted by some bacteria to kill competing bacteria. Cleavage of tRNAs is also associated with some stress responses in eukaryotic cells, but the cleavage does not significantly reduce cellular tRNA pools, and no tRNA-depleting ACNases have been reported for multicellular organisms. Thus, new compositions and methods are needed to treat diseases caused by SAMD9 and/or SAMD9L mutations.

### SUMMARY

The present disclosure provides herein methods of treating diseases caused by SAMD9 and/or SAMD9L mutations. Herein, it was found that human SAMD9 is a virus-activatable ACNase that specifically depletes phenylalanine tRNA (tRNA^{Phe}). SAMD9 ACNase can be activated by poxvirus infection or by SAMD9 mutations associated with a spectrum of developmental or immunological disorders, implicating tRNA^{Phe} deficiency as a major toxic effect in these human diseases and providing a new therapeutic strategy.

In one aspect, disclosed herein is a method of treating a pediatric myelodysplastic syndrome in a subject in need thereof, wherein the method comprises administering a pharmaceutically effective amount of a Sterile Alpha Motif Domain-containing 9 (SAMD9) inhibitor or a Sterile Alpha Motif Domain-containing 9 Like (SAMD9L) inhibitor. In one embodiment, the SAMD9 inhibitor or the SAMD9L inhibitor is a synthetic transfer RNA (tRNA). In some embodiments, the synthetic tRNA is a synthetic phenylalanine tRNA (tRNA^{Phe}) In other embodiments, the SAMD9 inhibitor or SAMD9L inhibitor decreases an activity of a SAMD9 gain of function (GoF) mutation or an activity of a SAMD9L GoF mutation. In another embodiments, the SAMD9 GoF mutation or the SAMD9L GoF mutation decreases protein synthesis in the subject.

In some embodiments, the pediatric myelodysplastic syndrome is any one of the following disorders selected from the group consisting of refractory cytopenia of childhood (RCC), refractory anemia with excess blasts (RAEB), and RAEB in transformation (RAEB-t).

In another aspect, disclosed herein is a method of treating an adult ataxia or an adult pancytopenia in a subject in need thereof, wherein the method comprises administering a pharmaceutically effective amount of a Sterile Alpha Motif Domain-containing 9 (SAMD9) inhibitor or a Sterile Alpha Motif Domain-containing 9 Like (SAMD9L) inhibitor. In one embodiment, the SAMD9 inhibitor or the SAMD9L inhibitor is a synthetic transfer RNA (tRNA). In some embodiments, the synthetic tRNA is a synthetic phenylalanine tRNA (tRNA^{Phe}).

In another embodiment, the SAMD9 inhibitor or SAMD9L inhibitor decreases an activity of a SAMD9 gain of function (GoF) mutation or an activity of a SAMD9L GoF mutation. In other embodiments, the SAMD9 GoF mutation or the SAMD9L GoF mutation decreases protein synthesis in the subject.

In some embodiments, the adult ataxia is any one of the following disorders selected from the group consisting of Friedreich's ataxia, Ataxia-telangiectasia, spinocerebellar ataxias, episodic ataxia, acquired ataxia, idiopathic late-onset cerebellar ataxia (ILOCA), and ataxia with vitamin E deficiency. In some embodiments, the adult pancytopenia comprises the subject presenting with any one of following blood diseases selected from the group consisting of anemia, leukopenia, and thrombocytopenia.

In another aspect, disclosed herein is a method of treating a pediatric myelodysplastic syndrome in a subject in need thereof, wherein the method comprises administering a vector comprising a phenylalanine tRNA (tRNA^{Phe}) gene. In some embodiments, the vector is a plasmid, a viral vector, or a nanoparticle. In some embodiments, the method increases protein synthesis in the subject.

In another embodiment, the pediatric myelodysplastic syndrome is any one of the following disorders selected from the group consisting of refractory cytopenia of childhood (RCC), refractory anemia with excess blasts (RAEB), and RAEB in transformation (RAEB-t).

In another aspect, disclosed herein is a method of treating an adult ataxia or an adult pancytopenia in a subject in need thereof, wherein the method comprises administering a vector comprising a phenylalanine tRNA (tRNA^{Phe}) gene. In some embodiments, the vector is a plasmid, a viral vector, or a nanoparticle. In some embodiments, the method increases protein synthesis in the subject.

In another embodiment, the adult ataxia is any one of the following disorders selected from the group consisting of Friedreich's ataxia, Ataxia-telangiectasia, spinocerebellar ataxias, episodic ataxia, acquired ataxia, idiopathic late-onset cerebellar ataxia (ILOCA), and ataxia with vitamin E deficiency. In other embodiments, the adult ataxia is any one of the following disorders selected from the group consisting of Friedreich's ataxia, Ataxia-telangiectasia, spinocerebellar ataxias, episodic ataxia, acquired ataxia, idiopathic late-onset cerebellar ataxia (ILOCA), and ataxia with vitamin E deficiency.

In some embodiments, the adult pancytopenia comprises the subject presenting with any one of following blood diseases selected from the group consisting of anemia, leukopenia, and thrombocytopenia.

In some embodiments, the subject is a human.

In one aspect, disclosed herein is an assay for screening a SAMD9 inhibitor or a SAMD9L inhibitor, wherein the assay comprises a synthetic RNA probe, wherein the synthetic RNA probe mimics a human tRNA-Phenylalanine (tRNA^{Phe}) anticodon stem loop.

In some embodiments, the synthetic RNA probe comprises at least 80% sequence identity to SEQ ID NO: 156. In some embodiments, the synthetic RNA probe comprises at least 90% sequence identity to SEQ ID NO: 156. In some embodiments, the synthetic RNA probe comprises SEQ ID NO: 156.

In some embodiments, the synthetic RNA probe comprises a dye molecule. In some embodiments, the dye molecule comprises a 6-carboxyfluorescein (6-FAM) dye. In some embodiments, the synthetic RNA probe comprises a quencher. In some embodiments, the quencher comprises an Iowa Black FQ (IABkFQ) quencher.

In some embodiments, the assay further comprises a cleavage buffer. In some embodiments, the assay is a Fluorescence Resonance Energy Transfer (FRET)-based assay. In some embodiments, the assay is a gel-based assay. In some embodiments, the assay further measures that activity of a SAMD9 tRNase or SAMD9L tRNase.

In another aspect, disclosed herein is a method of screening an inhibitor of a SAMD9 activity or a SAMD9L activity, wherein the method comprises using the assay of any preceding aspect. In one embodiment, the inhibitor is a tRNA, a siRNA, a mRNA, or a microRNA. In some embodiments, the SAMD9 activity or the SAMD9L activity is detected measuring a chemiluminescent signal, a fluorescent signal, or a colorimetric signal.

The present disclosure also provides a synthetic nucleic acid composition as a SAMD9 or SAMD9L inhibitor.

In another aspect, disclosed herein is a synthetic nucleic acid comprising a stem-loop with a SAMD9 cleavage site or a stem-loop with a SAMD9L cleavage site, wherein said nucleic acid further comprises at least 10 nucleotides. In one embodiment, the stem-loop comprises a stem segment with greater than 2 nucleotides. In another embodiment, the stem-loop comprises a loop segment with 7 or 8 nucleotides.

In other embodiments, the loop segment comprises a methylated purine at a third position of the loop segment. In other embodiments, the methylated purine is a 2′-O-methyl purine. In other embodiments, a pyrimidine is at a first position of the loop segment, and wherein said pyrimidine is a SAMD9 cleavage site or a SAMD9L cleavage site. In some embodiments, the pyrimidine is separated from the methylated purine by one nucleotide. In some embodiments, the synthetic nucleic acid is a SAMD9 inhibitor or a SAMD9L inhibitor.

### BRIEF DESCRIPTION OF FIGURES

The accompanying figures, which are incorporated in and constitute a part of this specification, illustrate several aspects described below.

FIGS. 1A-1E show that SAMD9 is a vaccinia virus (VACV)-activable endonuclease that specifically depletes tRNA^{Phe} with FTSJ-mediated 2′-O methylation. FIG. 1A shows that HeLa cells with a Dox-inducible WT FTSJ1 allele were induced to express different amount of FTSJ1. The abilities of cells to restrict a SAMD9-sensitive mutant VACV (vK1⁻C7⁻) were determined by measuring the viral titers at 0 and 24 hour post-infection (hpi). FIG. 1B shows that HeLa cells were engineered to express no FSTJ1 or different FTSJ1 mutants. FTSJ1-K28A mutant is catalytically inactive, while FTSJ1-A26P mutant is defective only at methylation of at position 34 of tRNA. Host restriction of vK1⁻C7⁻ was determined as in A. FIG. 1C shows that WT and ΔFTSJ1 HeLa cells were infected with vK1⁻C7⁻ for the indicated hour. 8+A, infection for 8 hours in the presence of AraC. Total RNA from HeLa cells were incubated with purified recombinant SAMD9¹³⁴⁻³⁸⁵ protein. RNAs were resolved on denaturing gel and stained with SYBR Gold. Northern blots against tRNA^{Phe} or tRNA^{Leu} were performed with two or one probes, respectively. The levels of the tRNA with respect to uninfected cells were quantified from the Northern. Arrows point to the cleavage products. FIG. 1D shows that total HeLa cell RNA was incubated with SAMD9¹³⁴⁻³⁸⁵ for the indicated time. FIG. 1E shows that total RNA from HeLa (WT) or ΔFTSJ1 (ΔFt1) cells were incubated with SAMD9¹³⁴⁻³⁸⁵ protein for one hour.

FIGS. 2A-2E show that putative tRNA binding and active site of SAMD9¹³⁴⁻³⁸⁵. FIG. 2A shows that SAMD9 residues were studied with mutagenesis are shown in the SAMD9 structure with DNA. Underlined residues are essential for SAMD9¹³⁴⁻³⁸⁵ cleavage of tRNA^{Phe} with cyan and red indicating putative binding and active site residues, respectively. FIG. 2B shows the effects of the indicated mutations on SAMD9¹³⁴⁻³⁸⁵ binding of yeast tRNA^{Phe} in a gel mobility shift assay. FIG. 2C shows that total HeLa cell RNAs were incubated with SAMD9¹³⁴⁻³⁸⁵ in the presence of different divalent cation for 1 hour. tRNA^{Phe} fragments were detected by Northern blot. FIG. 2D shows the effect of acidic residue mutations on SAMD9 activities. FIG. 2E shows the effects of the mutations on SAMD9¹³⁴⁻³⁸⁵ cleavage of human tRNA^{Phe}.

FIGS. 3A-3F show that SAMD9¹³⁴⁻³⁸⁵ is an anticodon nuclease with a unique substrate specificity. FIG. 3A shows that a 38-nt synthetic RNA was incubated with SAMD9¹³⁴⁻³⁸⁵ for one hour. The cleavage products were resolved on the gel and their MWs determined by mass spectrometry. The arrow points to the cleavage site. MWs of different fragments are listed. The XrnI symbol indicates that the 3′-end fragment can be degraded by XrnI. FIG. 3B shows the reaction was performed in the absence or presence of XrnI. The arrow points to the 3′ end product that disappeared in the presence of XrnI FIGS. 3C-3E shows that RNA with the indicated sequence were incubated with SAMD9¹³⁴⁻³⁸⁵ for 1 hour. The size of arrow next to the cleavage site indicates the cleavage efficiency. Positions where sequence differ from that of tRNA^{Phe} were highlighted. FIG. 3F shows the consensus RNA structure that is cleaved by SAMD9¹³⁴⁻³⁸⁵.

FIGS. 4A-4D show that SAMD9 activated by VACV or through a GoF mutation specifically depletes cellular tRNA^{Phe} and causes ribosomal pausing at Phe codons. FIG. 4A shows that tRNA-seq revealed a specific depletion of tRNA^{Phe} in vK1⁻C7⁻-infected HeLa cells with respect to WT VACV infected cells. FIG. 4B shows that SAMD9 causes ribosomal pausing at the Phe codons in infected cells. Global codon occupancy of all cellular transcripts was analyzed in HeLa cells infected with either vK1⁻C7⁻ or WT VACV. One set of infection was performed in the presence of an inhibitor of DNA replication to limit the infection to the early phase. The relative codon occupancy in vK1⁻C7⁻-infected cells versus WT VACV-infected showed an increase in ribosomal occupancy at Phe codons in the former cells. FIG. 4C shows that GoF SAMD9 causes the depletion of cellular tRNA^{Phe} but not tRNA^{Leu}. FIG. 4D shows that GoF SAMD9 causes ribosomal pausing at the Phe codons. Global codon occupancy of all cellular transcripts was analyzed in cells that were uninduced or induced to express SAMD9^{R1293W}.

FIGS. 5A-5D show that activated SAMD9 causes stress response associated with ATF3. FIG. 5A shows the transcriptome analysis of HeLa cells infected with different VACV. FIG. 5B shows the transcriptome analysis of HeLa cells infected with VACV shows a common set of 9 human genes that were induced by a GoF SAMD9 and by infection with the mutant VACV (but not the WT VACV). FIG. 5C shows the depletion of cellular tRNA^{Phe} and activation of ATF3 in VACV-infected cells in a SAMD9 and FTSJ1 dependent manner. HeLa cells with KO of SAMD9 and SAMD9L (AhSAMD9&L) or FTSJ1 (ΔFTSJ1) were infected with vK1⁻C7⁻ or WT VACV for 8 hours. tRNA^{Phe} and ATF3 level in the cells were determined by Northern blot and RT-qPCR, respectively.ATF3 level is related to 18S level. FIG. 5D shows that SAMD9^{R1293W} activates ATF3 and p38. FIG. 5E shows that knockdown of cellular tRNA^{Phe} induces ATF3 activation.

FIGS. 6A-6B show that overexpression of tRNA^{Phe} reduces SAMD9 activities. FIG. 6A shows the SAMD9 inhibition of global protein synthesis was reduced by tRNA^{Phe} overexpression. HEK 293T cells were transfected with mCherry-SAMD9^{R982C} and a plasmid expressing tRNA^{Phe} or tRNA^{Leu} and labeled with O-propargyl-puromycin (OPP) for 30 minutes. Representative flow cytometry plots of OPP level relative to cellular mCherry level are shown. Nascent protein synthesis levels in SAMD9^{R982C}-expressing cells relative to non-transfected cells from the same culture wells are derived from the flow cytometry. Each replicate and SD are shown. FIG. 6B shows that SAMD9 inhibitions of protein synthesis and viral replication were reduced by tRNA^{Phe} in a dose-dependent manner. HEK 293T cells were co-transfected with a plasmid expressing mCherry-SAMD9^{R982C} and a plasmid expressing tRNA^{Phe} or a mutant tRNA^{Pe} and infected with vK1⁻C7⁻/GFP⁺. Infection rates (GFP+%) amongst SAMD9-expressing and non-transfected control cells from the same culture well were simultaneously determined with flow cytometry. Representative flowcytometry plots are shown. The MFI of mCherry-SAMD9 in transfected cells and the relative infection rates between SAMD9-expressing and non-transfected cells are derived from the flow cytometry data. Each biological replicate and SD are shown.

FIG. 7 shows that SAMD9 is a virus-activated endonuclease with a specificity for tRNA^{Phe}. The effect of recombinant SAMD9¹³⁴⁻³⁸⁵ or VACV infection on seven cellular tRNAs were shown.

FIGS. 8A-8C show that tRNA binding and active site of SAMD9¹³⁴⁻³⁸⁵. FIG. 8A shows that representative flow cytometry plots of the antiviral assays on SAMD9 mutants. The effect of SAMD9 on protein synthesis is reflected by their MFI in the cells. FIG. 8B shows the total RNA staining showing the cleavage product by different SAMD9 mutants. FIG. 8C shows that dose dependent cleavage of tRNA^{Phe} by recombinant SAMD9¹³⁴⁻³⁸⁵ protein.

FIGS. 9A-9I show that SAMD9 is an anticodon nuclease with a unique substrate specificity. FIG. 9A shows that SAMD9¹³⁴⁻³⁸⁵ cleaves human and yeast tRNA^{Phe} between A31 and C32. Total HeLa RNA and purified yeast tRNAP^{Phe} were incubated with SAMD9¹³⁴⁻³⁸⁵. Bands were cut from the gel (labeled from 1 to 4) and their molecule weight were determined by mass spectrometry. The sequence and molecular weight of different fragments of human and yeast tRNA^{Phe} were also indicated. Modified nucleosides in human tRNA^{Phe} is shown in green, and the location of the 2′-O methylation is labeled with the red m. Sequences in yeast tRNA^{Phe} that differ from human tRNA^{Phe} are highlighted. FIG. 9B shows that SAMD9¹³⁴⁻³⁸⁵ cleaves synthetic RNA with the anticodon stem loop. RNA with the indicated structure were digested with SAMD9¹³⁴⁻³⁵. The molecular weights of digested products were determined by mass spectrometry. Sequences in RNAs that differ from human tRNA^{Phe} are highlighted. FIG. 9C shows that SAMD9¹³⁴⁻³⁸⁵ cleaves a 17-nt synthetic RNA with a stem-loop structure. RNA with the indicated structure were digested with SAMD9¹³⁴⁻³⁸⁵. The molecular weights of digested products were determined by mass spectrometry. FIG. 9D shows the summary of predicated and observed molecular weight of different RNA fragments shown in panel A-C. FIG. 9E shows that RNA or DNA with the indicated structure were digested with SAMD9¹³⁴⁻³⁸⁵ Deoxynucleotides in the sequence are shown in blue. X indicates no cleavage, while the size of the arrows corresponds to relative cleavage efficiency. FIGS. 9F-9I shows that SAMD9¹⁵⁶⁻³⁸⁵ and SAMD9L¹⁵⁸⁻³⁸⁸ can cleave yeast and human tRNA^{Phe} as well as a 38-nt synthetic RNA with 2′-O-methylation at the wobble base.

FIGS. 10A-10H show the tRNA-seq and Ribo-seq analyses. FIG. 10A shows that tRNA-seq revealed a specific depletion of tRNA^{Phe} in vK1⁻C7⁻ infected HeLa cells with respect to WT VACV infected cells. Data in FIG. 4A is shown again in comparison with additional controls. One step of tRNA-seq was performed with RNA that was not specifically demethylated. FIG. 10B shows the Ribo-seq of viral infected cells showed good 3-nt periodicity. FIG. 10C shows the Ribo-seq of BT20 cells showed good 3-nt periodicity. FIG. 10D shows the relative codon occupancy in VACV-infected cells versus uninfected HeLa cells showed an increase in ribosomal occupancy at Phe codons in vK1⁻C7⁻ infected cells. FIG. 10E-10H shows the effect of transient expression of different GoF/LoF SAMD9 on tRNA^{Phe} level measured by qPCR and Northern blot.

FIG. 11 shows the VACV transcriptome of cells infected with vK1⁻C7⁻ or WT VACV, in the presence or absence of AraC, are shown in the context of VACV genome. Gene expression in one area of the genome is enlarged. The lack of expression of K1L and C7L in the mutant is indicated. AraC limits WT and mutant to only early gene expression. The mutant has reduced expression in the late genes compared to WT.

FIGS. 12A-12C show the SAMD9/9L inhibitions of protein synthesis and viral replication were reduced by overexpressing tRNA^{Phe}. HEK 293T cells were co-transfected with a plasmid expressing mCherry-SAMD9/9L and a plasmid expressing tRNA^{Phe} or tRNA^{Leu} and infected with vK1⁻C7⁻/GFP*. Infection rates (GFP+%) amongst SAMD9/9L-expressing and non-transfected control cells from the same culture well were simultaneously determined with flow cytometry. FIG. 12A shows the representative flowcytometry plots and GFP histogram are shown. FIGS. 12B-12C shows the MFI of mCherry-SAMD9/9L in transfected cells and the relative infection rates between SAMD9-expressing and non-transfected cells are derived from the flow cytometry data. Each biological replicate and SD are shown. FIG. 12D shows that SAMD9^{R1293W} inhibitions of protein synthesis were reduced by overexpressing tRNA^{Phe} shown by puromycin labeling.

FIGS. 13A-13B show that 2′-O methylation at the wobble position is a conserved feature for eukaryotic but not prokaryotic tRNA^{Phe}. a). The pie chart categorizes the 38 species with known tRNA^{Phe} modification status in the RNA modification database. 17 of the 19 species with 2′-O methylation at G34 (Gm34, left) are eukaryotes, while 18 of the 19 species with no such modification (G34, right) are prokaryotes or of prokaryotic origins. b). Phylogenetic tree of the 38 species was retrieved from timetree.org. The species with 2′-O methylation at the wobble position of tRNA^{Phe} have their name labeled green. MYA: million years ago.

FIGS. 14A-14C show the fluorescence signal depends on wild type (WT) SAMD and the substrate being methylated. FIG. 14A shows the fluorescent substrate. FIG. 14B shows that the signal depends on the WT SAMD9. FIG. 14C shows that the signal depends on the substrate being methylated.

FIGS. 15A-15H show that the initial velocity depends on enzyme concentration and substrate concentrations.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the disclosure described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the disclosure, examples of which are illustrated in the drawings and the examples. This disclosure may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising,” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a,” “an,” “the,” include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising,” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Amino acid” is used herein to refer to a chemical compound with the general formula: NH₂—CRH COOH, where R, the side chain, is H or an organic group. Where R is organic, R can vary and is either polar or nonpolar (i.e., hydrophobic). The following abbreviations are used throughout the application: A=Ala=Alanine, T=Thr=Threonine, V=Val=Valine, C=Cys=Cysteine, L=Leu=Leucine, Y=Tyr=Tyrosine, I=Ile=Isoleucine, N=Asn=Asparagine, P=Pro=Proline, Q=Gln=Glutamine, F=Phe=Phenylalanine, D=Asp=Aspartic Acid, W=Trp=Tryptophan, E=Glu=Glutamic Acid, M=Met=Methionine, K=Lys=Lysine, G=Gly=Glycine, R=Arg=Arginine, S=Ser=Serine, H=His=Histidine. Unless otherwise indicated, the term “amino acid” as used herein also includes amino acid derivatives that nonetheless retain the general formula.

A “promoter,” as used herein, refers to a sequence in DNA that mediates the initiation of transcription by an RNA polymerase. Transcriptional promoters may comprise one or more of a number of different sequence elements as follows: 1) sequence elements present at the site of transcription initiation; 2) sequence elements present upstream of the transcription initiation site and; 3) sequence elements down-stream of the transcription initiation site. The individual sequence elements function as sites on the DNA, where RNA polymerases and transcription factors facilitate positioning of RNA polymerases on the DNA bind.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists.

As used herein, by a “subject” means an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

A “gene” refers to a polynucleotide containing at least one open reading frame that is capable of encoding a particular polypeptide or protein after being transcribed and translated.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, palliatively, or remedially. Treatments are administered to a subject prior to onset (e.g., before obvious signs of disease), during early onset (e.g., upon initial signs and symptoms of disease), or after an established development of disease.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material. A nucleic acid can also be a transfer RNA (tRNA), a messenger RNA, a small interfering RNA (siRNA), a short hairpin RNA (shRNA), or a microRNA (miRNA).

As used herein, a “mutation” refers to changing the structure of a gene, resulting in a variant form that may be transmitted to later generations. A mutation can be caused by the alteration of single nucleotides in DNA, or the deletion, insertion, or rearrangement of larger sections of genes. A mutation can lead to the expression of a protein that has been changed physically or functionally leading to lethality, non-lethal dysfunction effects, or no effects. For example, a “gain of function” or “GoP” mutation refers to a type of mutation in which the altered gene produces a product (protein, polypeptide, or peptide) that possesses new molecular functions or a new pattern of gene expression. These new functions or expression patterns caused by GoF mutations usually enhance or increase the innate functions, whether they be inhibitory or activating.

The term “detect” or “detecting” refers to an output signal released for the purpose of sensing of physical phenomenon. An event or change in environment is sensed and signal output released in the form of light or a change in color.

A “vector” refers to a DNA composition used as a vehicle to artificially carry foreign genetic material into another cell or tissue. A vector can be a DNA vector, bacterial vector, a viral vector, or a nanoparticle. A “DNA plasmid” is a circular DNA molecule (usually of the same species as the subject) that is genetically engineered to carry therapeutic genes into a subject's cells. A “bacterial vector” is a small extrachromosomal DNA molecule that has been engineered to not caused infection, and that can be incorporated into another cell that is physically separated from the chromosomal DNA and is easily replicated. A viral vector is a vehicle originally from a non-infectious virus used to artificially carry foreign genetic material into another cell or tissue.

### Methods of Treatment

Sterile Alpha Motif Domain-containing 9 (SAMD9) and SAMD9-Like (SAMD9L) are tumor suppressors and antiviral factors located on human chromosome 7. A myriad of human diseases including many pediatric myelodysplastic syndromes are caused by mutations in SAMD9 and/or SAMD9L. SAMD9 and SAMD9L are endonucleases that specifically cleave, or breakdown, tRNA nucleic acids. More specifically, SAMD9 and SAMD9L are anticodon nucleases (ACNases) that cleaves tRNA, specifically within the loop segment comprising the anticodon sequence. tRNAs are small RNA molecules that are essential for protein synthesis that serve as link between messenger RNA (mRNA) and newly synthesized amino acids that make up a protein or peptide. Each tRNA molecule comprises a three nucleotide code (or anticodon) that is complementary to an mRNA code (codon). Each tRNA molecule also decodes a specific amino acid, including phenylalanine, alanine, valine, isoleucine, leucine, methionine, tyrosine, tryptophan, serine, threonine, asparagine, glutamine, cysteine, glycine, proline, arginine, histidine, lysine, aspartate, and glutamate. SAMD9 and SAMD9L ACNases have further specificity of cleaving phenylalanine tRNA (tRNA^{Phe}).

In normal settings, SAMD9 and SAMD9L ACNase activities are induced by viral infection and enhanced by patient derived mutations to disrupt protein synthesis. SAMD9 and/or SAMD9L gain of function (GoF) mutations, as seen in pediatric myelodysplastic syndromes and adult ataxias, impede or block cell growth by exacerbating inhibited protein synthesis. Thus, there is a need to develop methods for targeting SAMD9 and/or SAMD9L in human diseases caused by SAMD9 and/or SAMD9L GoF mutations.

The present disclosure provides methods of treating diseases caused by SAMD9 and/or SAMD9L mutations.

In one aspect, disclosed herein is a method of treating a pediatric myelodysplastic syndrome in a subject in need thereof, wherein the method comprises administering a pharmaceutically effective amount of a Sterile Alpha Motif Domain-containing 9 (SAMD9) inhibitor or a Sterile Alpha Motif Domain-containing 9 Like (SAMD9L) inhibitor. In one embodiment, the SAMD9 inhibitor or the SAMD9L inhibitor is a synthetic tRNA. In some embodiments, the synthetic tRNA is a synthetic phenylalanine tRNA (tRNA^{Phe}).

In other embodiments, the SAMD9 inhibitor or SAMD9L inhibitor decreases an activity of a SAMD9 gain of function (GoF) mutation or an activity of a SAMD9L GoF mutation. In another embodiments, the SAMD9 GoF mutation or the SAMD9L GoF mutation decreases protein synthesis in the subject.

In some embodiments, the pediatric myelodysplastic syndrome is any one of the following disorders selected from the group consisting of refractory cytopenia of childhood (RCC), refractory anemia with excess blasts (RAEB), and RAEB in transformation (RAEB-t), and pediatric bone marrow failure syndromes.

In another aspect, disclosed herein is a method of treating an adult ataxia or an adult pancytopenia in a subject in need thereof, wherein the method comprises administering a pharmaceutically effective amount of a Sterile Alpha Motif Domain-containing 9 (SAMD9) inhibitor or a Sterile Alpha Motif Domain-containing 9 Like (SAMD9L) inhibitor. In one embodiment, the SAMD9 inhibitor or the SAMD9L inhibitor is a synthetic transfer RNA (tRNA). In some embodiments, the synthetic tRNA is a synthetic phenylalanine tRNA (tRNA^{Phe}).

In another embodiment, the SAMD9 inhibitor or SAMD9L inhibitor decreases an activity of a SAMD9 gain of function (GoF) mutation or an activity of a SAMD9L GoF mutation. In other embodiments, the SAMD9 GoF mutation or the SAMD9L GoF mutation decreases protein synthesis in the subject.

In some embodiments, the adult ataxia is any one of the following disorders selected from the group consisting of Friedreich's ataxia, Ataxia-telangiectasia, spinocerebellar ataxias, episodic ataxia, acquired ataxia, idiopathic late-onset cerebellar ataxia (ILOCA), and ataxia with vitamin E deficiency.

In some embodiments, the adult pancytopenia comprises the subject presenting with any one of following blood diseases selected from the group consisting of anemia, leukopenia, and thrombocytopenia.

Gene therapy techniques are utilized to modify a subject's genes to treat or cure a disease. These therapies work by replacing a disease-causing gene with a healthy copy of the gene, inactivating a disease-causing gene, and/or introducing a new or modified gene to the subject to help treat the disease. Delivery of genetic material to a subject can be carried in any one of the following methods, including, but not limited to plasmid DNA vectors, viral vectors, bacterial vectors, human genome editing (such as CRISPR Cas systems), or patient-derived cellular gene therapy products. Another gene therapy method comprises genetic material encapsulated in a nanoparticle for delivery into a subject's cells.

In another aspect, disclosed herein is a method of treating a pediatric myelodysplastic syndrome in a subject in need thereof, wherein the method comprises administering a vector comprising a phenylalanine tRNA (tRNA^{Phe}) gene. In some embodiments, the vector is a DNA plasmid, a bacterial vector, a viral vector, or a nanoparticle. In some embodiments, the method comprises a genome editing system to deliver tRNAs to a subject. In some embodiments, the method comprises a gene editing system to deliver tRNA^{Phe} to a subject. In some embodiments, the method increases protein synthesis in the subject.

In another embodiment, the pediatric myelodysplastic syndrome is any one of the following disorders selected from the group consisting of refractory cytopenia of childhood (RCC), refractory anemia with excess blasts (RAEB), and RAEB in transformation (RAEB-t).

In another aspect, disclosed herein is a method of treating an adult ataxia or an adult pancytopenia in a subject in need thereof, wherein the method comprises administering a vector comprising a phenylalanine tRNA (tRNA^{Phe}) gene. In some embodiments, the vector is a DNA plasmid, a bacterial vector, a viral vector, or a nanoparticle. In some embodiments, the method comprises a genome editing system to deliver tRNAs to a subject. In some embodiments, the method comprises a gene editing system to deliver tRNA^{Phe} to a subject. In some embodiments, the method increases protein synthesis in the subject.

In another embodiment, the adult ataxia is any one of the following disorders selected from the group consisting of Friedreich's ataxia, Ataxia-telangiectasia, spinocerebellar ataxias, episodic ataxia, acquired ataxia, idiopathic late-onset cerebellar ataxia (ILOCA), and ataxia with vitamin E deficiency. In other embodiments, the adult ataxia is any one of the following disorders selected from the group consisting of Friedreich's ataxia, Ataxia-telangiectasia, spinocerebellar ataxias, episodic ataxia, acquired ataxia, idiopathic late-onset cerebellar ataxia (ILOCA), and ataxia with vitamin E deficiency.

In some embodiments, the adult pancytopenia comprises the subject presenting with any one of following blood diseases selected from the group consisting of anemia, leukopenia, and thrombocytopenia. In some embodiments, the subject is a human.

Understanding an enzyme's activity is essential for drug screening and development. During a screening for inhibitors or activators of a target enzyme, the target enzyme is initially exposed to a starting material, or substrate, and its activity is determined by the disappearance of the substrate or the appearance of a product, or byproduct. Addition of an inhibitor or activator will decrease or increase the enzyme's activity, respectively, with the best inhibitors or activators being further tested against disease. Therefore, screening for inhibitors of SAMD9 and/or SAMD9L provides methods of identifying drug molecules to treat diseases caused by SAMD9 and/or SAMD9L mutations.

### Screening Assays and Methods of Use

In one aspect, disclosed herein is an assay for screening SAMD9 or SAMD9L inhibitor, wherein the assay comprises a synthetic RNA probe, wherein the synthetic RNA probe mimics a human tRNA-Phenylalanine (tRNA^{Phe}) anticodon stem loop.

In some embodiments, the synthetic RNA probe comprises at least 70% sequence identity to SEQ ID NO: 156. In some embodiments, the synthetic RNA probe comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to SEQ ID NO: 156.

In some embodiments, the synthetic RNA probe comprises a dye molecule. In some embodiments, the dye molecule includes, but is not limited to a 6-carboxyfluorescein (6-FAM) dye, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4- I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO- TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis- BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow: Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD- Lipophilic Tracer; DiD (DilC18(5)); DIDS; Dihydorhodamine t23 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (1t) chloride; EYFP; Fast Blue; FDA; Feulgen (Pararosaniline); FIF (Formaldehyde Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO- I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy- N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1;YOYO-3; Sybr Green; Thiazole orange (interchelating dyes); semiconductor nanoparticles such as quantum dots; or a combination thereof.

In some embodiments, the synthetic RNA probe comprises a quencher. In some embodiments, the quencher includes, but is not limited to an Iowa Black FQ (IABkFQ) quencher, an Iowa Black RQ quencher, a BlackHole quencher (BHQ), such as for example a BHQ-1, a BHQ-2, and a BHQ-2; a Blackberry Quencher (BBQ), such as for example a BBQ-650; an Atto quencher, such as, for example Atto 540Q, Atto 575Q, and Atto 612Q; a Dabcyl quencher, such as for example a Dabcyl quencher deoxythymidine (dT), Dabcyl Quencher-3′, and Dabcyl Quencher-5′; an MGB-5′ probe, an MGB-3′ probe, or a Tamra quencher, such as for example Tamra NHS, TAMRA-3′, and TAMRA-dT.

In some embodiments, the synthetic RNA probe comprises a dye molecule of any preceding aspect and a quencher of any preceding aspect. In some embodiments, the dye molecule is located on the 5′ end of the stem loop of the RNA probe. In some embodiments, the dye molecule is located on the 3′ end of the stem loop of the RNA probe. In some embodiments, the quencher is located on the 3′ end of the stem loop of the RNA probe. In some embodiments, the quencher is located on the 5′ end of the stem loop of the RNA probe.

In some embodiments, the assay further comprises a cleavage buffer. In some embodiments, the assay further comprises an activity buffer or a reaction buffer. In some embodiments, the assay further comprises a storage buffer. In some embodiments, the assay further comprises a stabilizing buffer. In some embodiments, the assay further comprises a dissolving buffer. In some embodiments, the assay is a Fluorescence Resonance Energy Transfer (FRET)-based assay. In some embodiments, the assay is an in vitro assay. In some embodiments, the assay is a gel-based assay.

In some embodiments, the assay further measures that activity of a SAMD9 tRNase or SAMD9L tRNase. In some embodiments, the assay comprises a high-throughput screening assay for inhibitors of SAMD9 and/or SAMD9L. In some embodiments, the assay of any preceding aspect screens at least 10 or more small molecules. In some embodiments, the assay of any preceding aspect screens 10, 100, 1000, 10000, or more small molecules. In some embodiments, the assay of any preceding aspect screens more than 20,000 small molecules. In some embodiments, the assay of any preceding aspect screens more than 30,000 small molecules. In some embodiments, the small molecules of any preceding aspect include, but are not limited to tRNAs, siRNAs, mRNAs, and/or microRNAs (miRNAs).

In another aspect, disclosed herein is a method of screening an inhibitor of SAMD9 activity or SAMD9L activity, wherein the method comprises using the assay of any preceding aspect.

In another aspect, disclosed herein is a method of screening an inhibitor of a SAMD9 activity or a SAMD9L activity, wherein the method comprises preparing a solution comprising a SAMD9 enzyme, a SAMD9L enzyme, or any combination thereof, combining the inhibitor with any solution of the preceding step, and measuring the SAMD9 activity or the SAMD9L activity.

Blocking the expression of mutation SAMD9 or SAMD9L genes is essential for treating disease, therefore the present disclosure describes small interfering nucleic acids as inhibitors to bind to SAMD9 or SAMD9L mutant genes to prevent expression. In one embodiment, the inhibitor is a tRNA, a siRNA, a mRNA, or a microRNA. In some embodiments, the SAMD9 activity or the SAMD9L activity is detected measuring a chemiluminescent signal, a fluorescent signal, or a colorimetric signal.

tRNA Compositions and Compounds

Transfer RNA (tRNA) molecules have a distinctive folded nucleic acid structure with three hairpin loops attached to a central stem structure that form the shape of a three-leaf clover. One of the three stem loops comprise the three nucleotide anticodon that is complementary to the mRNA codon. The central stem structure, located opposite the anticodon, holds to amino acid specific to the tRNA. When the anticodon of the tRNA binds the codon of the mRNA, the tRNA transfers the amino acid to initiate or continue synthesis of a peptide molecule. Thus, tRNA are essential for protein synthesis. When SAMD9 and/or SAMD9L is expressed normally or with a GoF mutation, SAMD9L and/or SAMD9L decrease protein synthesis by cleaving tRNA molecules, specifically tRNA^{Phe}. Thus, there is a need to develop a synthetic tRNA molecule that binds and inhibits SAMD9 and/or SAMD9L activity in pediatric and adult diseases affected by SAMD9 and/or SAMD9L mutations.

The present disclosure also provides a synthetic nucleic acid composition as a SAMD9 or SAMD9L inhibitor.

In another aspect, disclosed herein is a synthetic nucleic acid comprising a stem-loop with a SAMD9 cleavage site or a stem-loop with a SAMD9L cleavage site, wherein said nucleic acid further comprises at least 10 nucleotides. In some embodiments, the nucleic acid comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides.

In one embodiment, the stem-loop comprises a stem segment with greater than 2 nucleotides. In one embodiment, the stem-loop comprises a stem segment with 2, 3, 4, 5, 6, 7, 8, 9, 10, 11, 12, 13, 14, 15, 16, 17, 18, 19, 20, or more nucleotides. In another embodiment, the stem-loop comprises a loop segment with 7 or 8 nucleotides. In another embodiment, the stem-loop comprises a loop segment with 5, 6, 7, 8, 9, 10, or more nucleotides.

In other embodiments, the loop segment comprises a methylated purine at a third position of the loop segment. In other embodiments, the methylated purine is a 2′-O-methyl purine. In other embodiments, a pyrimidine is at a first position of the loop segment, and wherein said pyrimidine is a SAMD9 cleavage site or a SAMD9L cleavage site. In some embodiments, the pyrimidine is separated from the methylated purine by one nucleotide. In some embodiments, the synthetic nucleic acid is a SAMD9 inhibitor or a SAMD9L inhibitor.

It should be understood that a human SAMD9 was found to be a virus activatable ACNase that specifically depletes phenylalanine tRNA (tRNAP^{Phe}). SAMD9 ACNase can be activated by viral infection or by SAMD9 mutations associated with a spectrum of developmental or immunological disorders, showing tRNAP^{Phe} deficiency as a major toxic effect in these human diseases and serves as a candidate for therapeutic strategies. In some embodiments, the viral infection is a poxvirus infection.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present disclosure which are apparent to one skilled in the art.

### Example 1. SAMD9/SAMD9L as an ACNase of Phenylalanine tRNAs

Transfer RNAs (tRNAs) are one of the most conserved components of protein synthesis machinery. An effective strategy for microbes to defend against viruses or competitors is to deplete specific tRNAs and shut off protein synthesis with anticodon nucleases (ACNases), but this strategy has not been observed in multicellular organisms. Human SAMD9 is a virus-activatable ACNase that specifically depletes phenylalanine tRNA (tRNA^{Phe}), causing codon-specific ribosomal pausing and inducing stress signaling. SAMD9 ACNase can be activated by poxvirus infection or by SAMD9 mutations associated with a spectrum of developmental or immunological disorders, revealing tRNA^{Phe} deficiency as a major toxic effect in these human diseases and presenting a new therapeutic strategy. The specificity of the ACNase, located to a N-terminal SAMD9 effector domain, is largely determined by a eukaryotic tRNA^{Phe} specific post-transcriptional modification at the wobble position, making eukaryotic tRNA^{Phe} a universal but specific target for SAMD9. The SAMD9 ACNase differs from the microbial ACNases in structure and substrate specificity, showing convergent evolution resulted in a universal immune defense strategy targeting tRNAs.

### Example 2. Human SAMD9 Mediates the Antiviral and Antiproliferative Activities by Acting as a tRNA^{Phe} Endoribonuclease

In vertebrate animals, interferon-regulated innate immune system serves as the first line of defense against viruses. Innate antiviral factors can pose as host restriction factors that block the replication of specific groups of viruses. Two mammalian interferon-stimulated gene products, SAMD9 and SAMD9L (SAMD9/9L), are restriction factors for poxviruses, which in turn have developed inhibitors of SAMD9/9L. Poxvirus mutants lacking the SAMD9/9L inhibitors fail to replicate in mammalian cells, due to a block in protein synthesis. In addition to their antiviral activities, SAMD9/9L also possess antiproliferative activities, particularly in myeloid cells. Germline mutations in SAMD9/9L cause a range of human diseases, including nearly 10% of the inherited bone marrow failure syndromes and a rare multi-system developmental disorder. The mutations are mostly missense mutations that result in a gain of function (GoF) in inhibition of cell proliferation and protein synthesis.

SAMD9/9L are thought to be members of the Signal Transduction ATPases with Numerous Domains (STAND) superfamily of immune sensors with a characteristic tripartite domain architecture, consisting of a C-terminal sensor that recognizes a stress-associated molecular pattern, a central Nucleotide-binding, and Oligomerization Domain (NOD) that mediates the formation of a supramolecular complex, and an N-terminal effector that is allosterically activated to exert the downstream effect. Consistently, an N-terminal effector domain was recently identified in SAMD9/9L, and its functions were found to be closely linked to its ability to bind double-stranded nucleic acid (dsDNA). In this example, SAMD9 inhibition of viral replication and cell proliferation through its effector domain was discovered, which unexpectedly shares a functional connection with microbial ACNases.

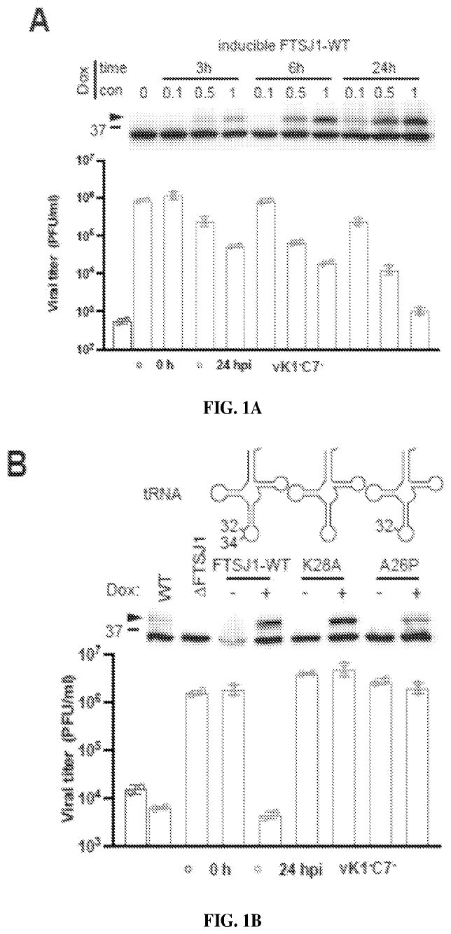

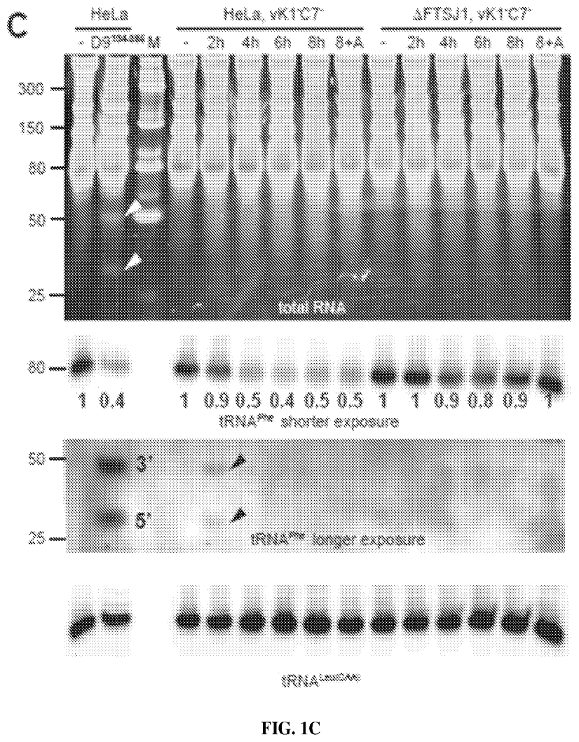

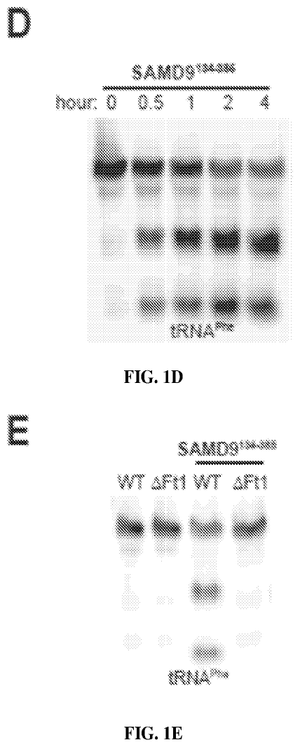

FTSJ1-mediated 2′-O methylation of tRNA wobble position is critical for host restriction of a SAMD9-sensitive vaccinia virus mutant. SAMD9, WDR6, and FTSJ1 were previously identified by a human genome-wide RNA interference screen as host restriction factors for vK1⁻C7⁻, a vaccinia virus (VACV) mutant that lacks both viral SAMD9/9L inhibitors. However, how they are connected in restricting VACV was unknown. FTSJ1 is an RNA methyltransferase that interacts separately with THADA and WDR6 to install 2′-O-ribose methylation at positions 32 and 34 of a few tRNA species, respectively. The co-identification of WDR6 but not THADA from the same genome-wide screen shows that tRNA methylation at position 34 is important for the host restriction. To test this FTSJ1 was knocked out from HeLa cells and the FTSJ1 KO cells (ΔFTSJ1) were reconstituted with a Dox-inducible FTSJ1 allele. FTSJ1 deficient cells failed to restrict vK1⁻C7⁻. Induction of FTSJ1-WT expression to different levels restored the restriction in a dose-dependent manner (FIG. 1A). By contrast, FTSJ1 with K28A or A26P mutation failed to restore the restriction (FIG. 1B). The K28A mutation of the catalytic site completely inactivates the methyltransferase activity, while the A26P mutation specifically disrupts methylation at position 34 but leaves methylation at position 32 intact. These results show that 2′-O methylation at position 34—the wobble position of the anticodon—is critical for host restriction of vK1⁻C7⁻.

SAMD9 is a VACV-activatable endonuclease with an exquisite specificity for 2′-O-methylated tRNA^{Phe}. In human cells, tRNA^{Phe}, tRNA^{Trp}, and tRNA^{Leu(CAA)} are known to be methylated at position 34 by FTSJ1. Among them, tRNA^{Phe} has been identified as the most biologically important substrate for FTSJ1 across eukaryotes. The functional connection between SAMD9 and FTSJ1/WDR6 was examined in host restriction of vK1⁻C7⁻ using tRNA^{Phe}. WT and ΔFTSJ1 HeLa cells were infected with vK1⁻C7⁻ and analyzed tRNA^{Phe} by Northern blot (FIG. 1C). In WT HeLa cells, tRNA^{Phe} level decreased continuously during the first few hours of infection, reaching a steady state of 40-50% of the original level by 4-6 hour post infection (hpi). Remarkably, two tRNA^{Phe} fragments appeared at 2 hpi when full-length tRNA^{Phe} level was still relatively high, showing that tRNA^{Phe} was cleaved into two fragments before being completely degraded. The degradation only requires the early phase of viral replication, as it was not affected when replication was blocked at genome replication by cytosine arabinoside (AraC). In ΔFTSJ1 cells, tRNA^{Phe} level was also reduced after vK1⁻C7⁻ infection, but the degree of reduction was relatively small. tRNA^{Phe} was not degraded in HeLa cells after WT VACV infection or in SAMD9 and SAMD9L KO (ASAMD9&L) cells after vK1⁻C7⁻ infection, showing that SAMD9 or SAMD9L is required for the degradation.

An effector domain in SAMD9/9L that binds to double stranded (ds) nucleic acids was identified. Remarkably, the incubation of recombinant SAMD9¹³⁴⁻³¹⁵ protein with total RNA from HeLa cells produced two distinct RNA fragments, which were abundant enough to be visualized with only nucleic acid staining (FIG. 1C). Northern blot identified the large (~50 nt) and the small (~30 nt) fragments to be 3′- and 5′-end of tRNA^{Phe}, respectively. They were identical in size to the two tRNA^{Phe} fragments transiently present in infected cells. With increasing incubation time, an increasing amount of tRNA^{Phe} was cleaved by SAMD9¹³⁴⁻³⁸⁵, but the cleavage products were not degraded (FIG. 1D).

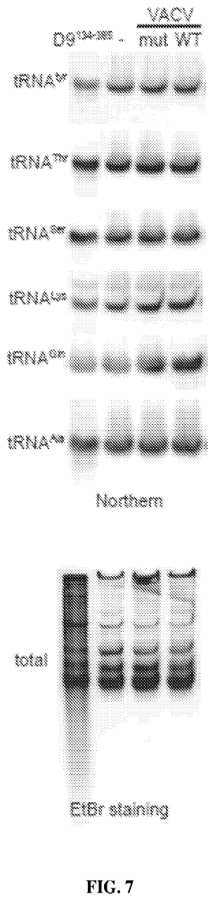

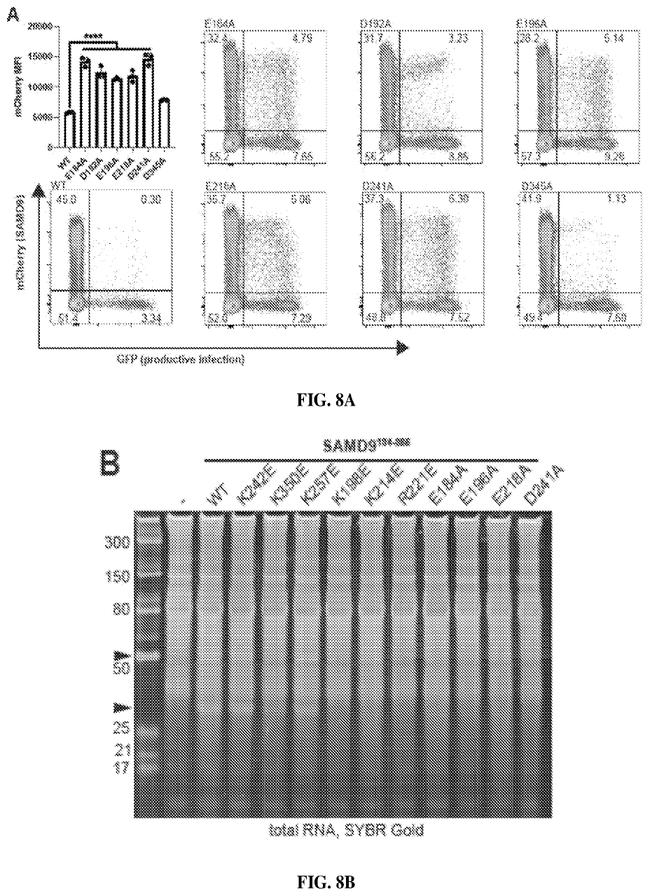

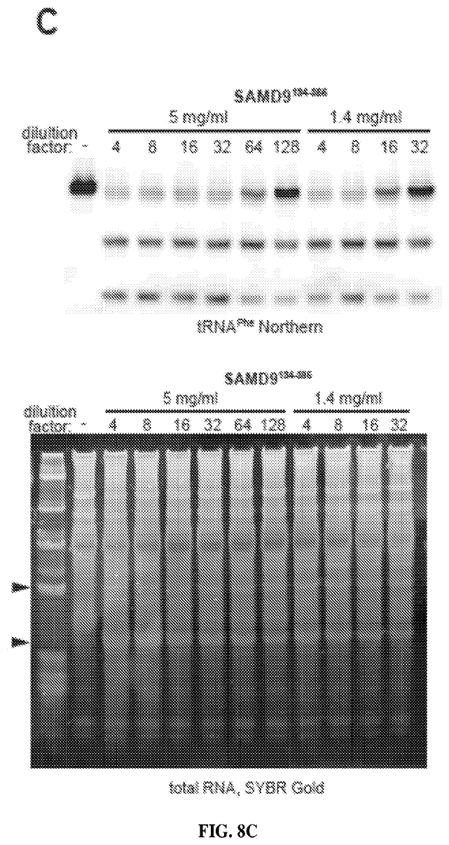

Notably, tRNA^{Leu(CAA)} as well as six other tRNAs were not cleaved by SAMD9¹³⁴⁻³⁸⁵ or degraded in cells infected with vK1⁻C7⁻ (FIG. 1C & FIG. 7). Importantly, SAMD9¹³⁴⁻³⁸ did not cleave tRNA^{Phe} from ΔFTSJ1 cells (FIG. 1E), supporting an exquisite specificity for 2′-O methylated tRNA^{Phe}.

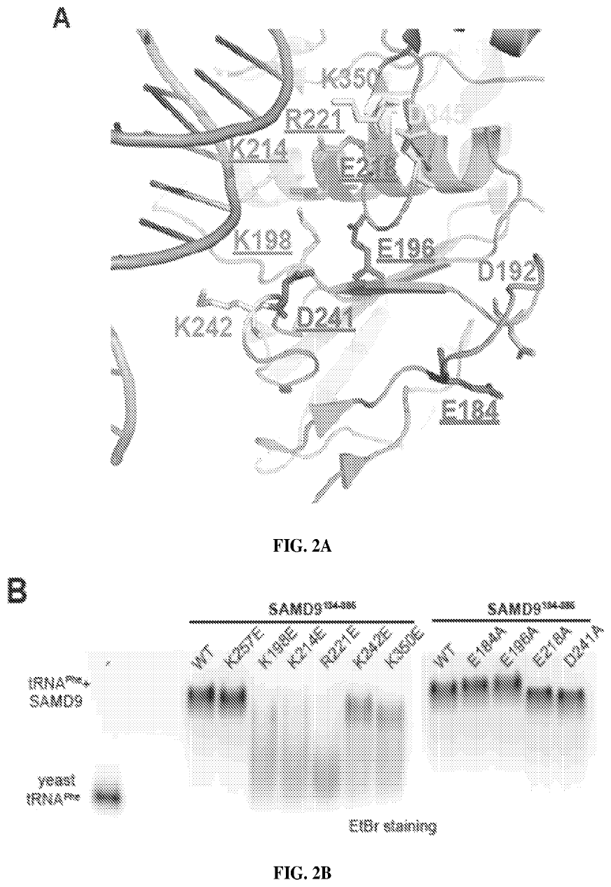

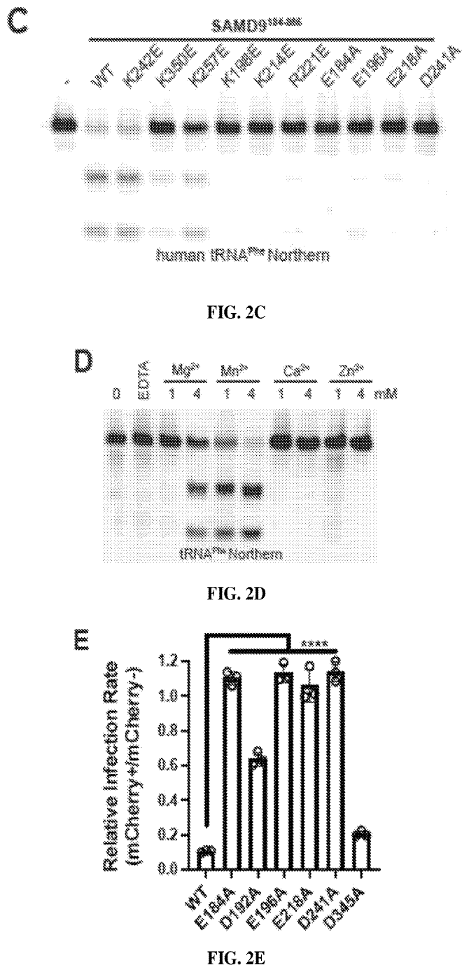

tRNA binding and active site of SAMD9¹³⁴³⁸⁵. The structure of SAMD9¹⁵⁶⁻³⁸⁵ was crystalized in complex with DNA and identified a dsDNA binding site that is essential for antiviral and antiproliferative activities of SAMD9 (FIG. 2A). With a gel mobility shift assay, SAMD9¹³⁴⁻³⁸⁵ was found to form a stable complex with purified yeast tRNA^{Phe}, and the binding is mediated by the previously identified dsDNA binding residues, including K198, K214 and R221. Specifically, K198E, K214E and R221E mutations abolished while K350E and K242E mutations reduced the binding (FIG. 2B). Consistently, these mutations affected the cleavage of human tRNA^{Phe} by SAMD9¹³⁴⁻³⁸⁵ to similar degrees (FIG. 2C).

A divalent cation is required for SAMD9¹⁵⁶⁻³⁸⁵ cleavage of tRNA^{Phe}. While Mn²⁺ is preferred over Mg²⁺, Ca²⁺ and Zn²⁺ do not support the cleavage of tRNA^{Phe} (FIG. 2D). This rather stringent selection of divalent cation is a characteristic of nucleases that use two metal ions, coordinated by acidic residues, for catalysis. Close examination of the structure of SAMD9¹⁵⁶⁻³⁸⁵/dsDNA complex located a cluster of conserved acidic residues near the dsDNA binding site. To test if this cluster could represent the active site, mutagenesis studies of conserved acidic residues near the dsDNA binding site was performed. Out of the six acidic residues, four residues (E184, E196, E218, and D241) were found as essential for SAMD9 antiviral activity, while D192 was partially required (FIG. 2D). These four acidic residues were not required for SAMD9¹³⁴⁻³⁸⁵ to bind but essential for it to cleave tRNA^{Phe} (FIG. 2B & 2E), showing that they are the active site residues of SAMD9.

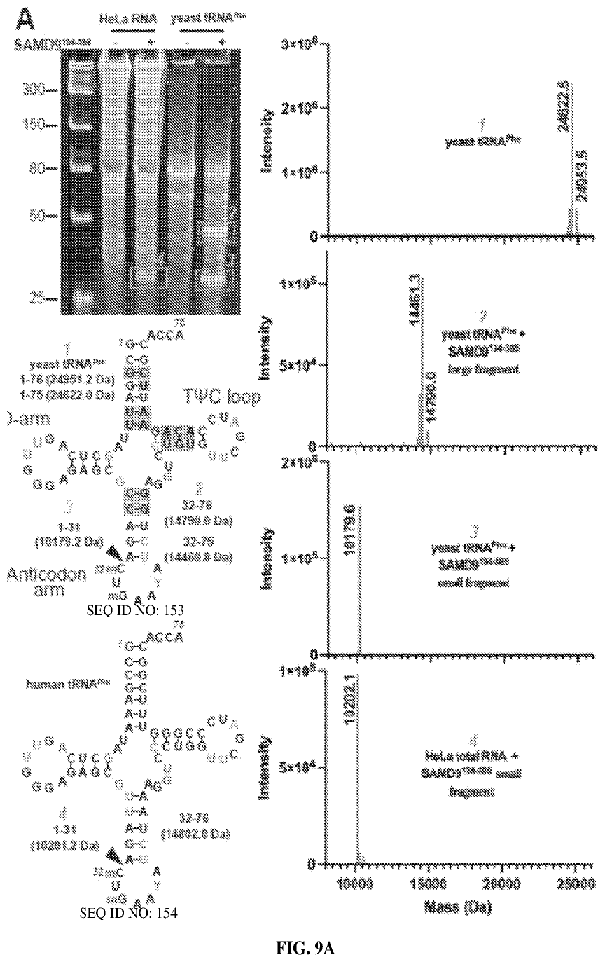

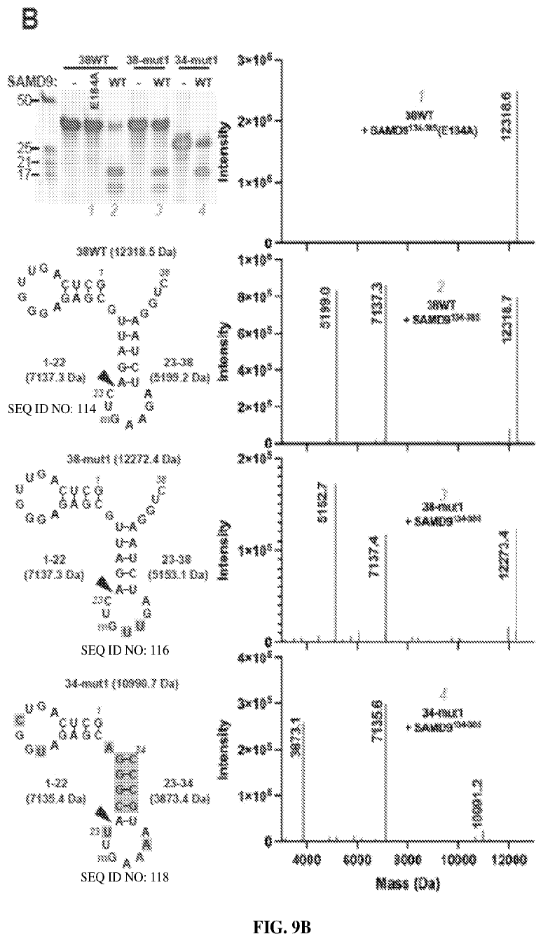

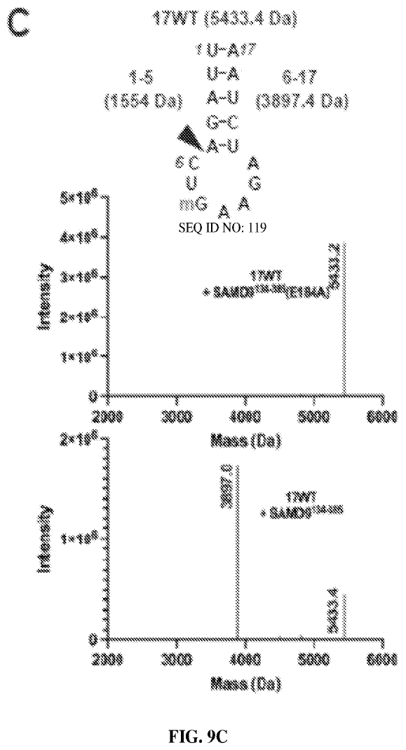

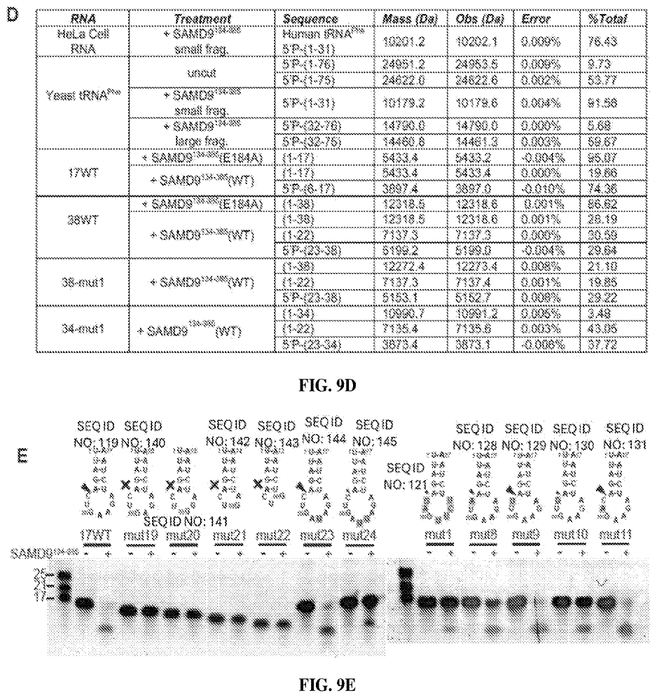

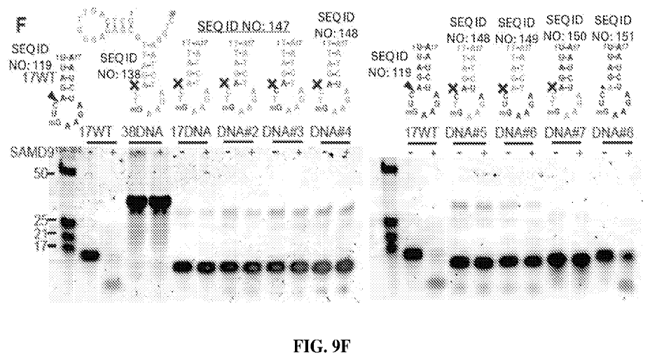

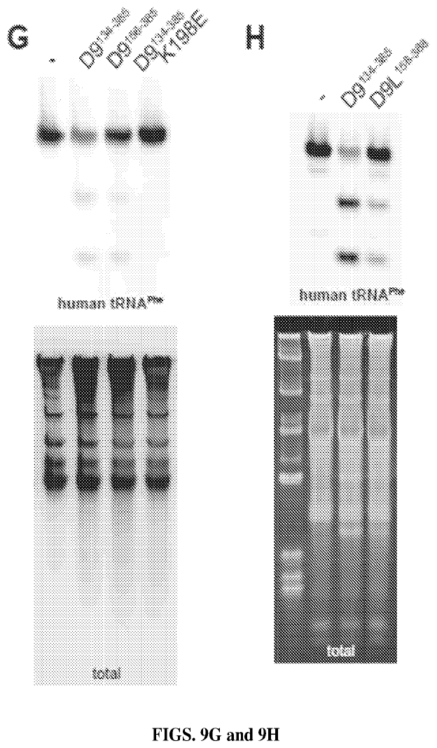

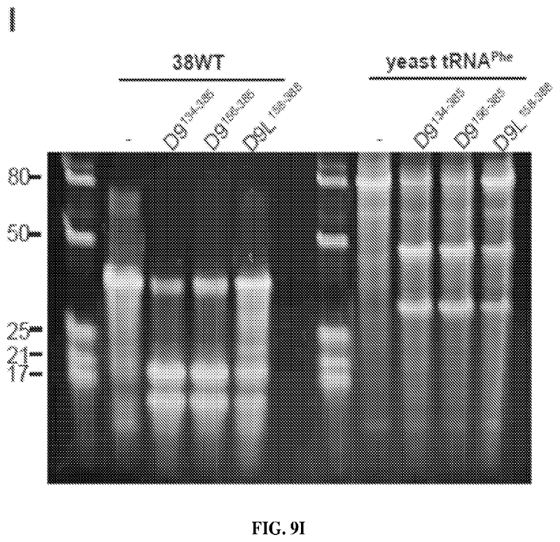

SAMD9¹³⁴⁻³⁸⁵ targets the anticodon stem loop with a 2′-O-methylguanosine. While SAMD9¹³⁴⁻³¹⁵ forms a stable complex with yeast tRNA^{Phe} at 4° C., it was surprising to find it cleaving yeast tRNA^{Phe} at 37° C. To identify the cleavage site, the cleavage products were isolated. Then, their molecular weights (MW) were determined by mass spectrometry (FIG. 9A). This analysis revealed that SAMD9¹³⁴⁻³⁸⁵ cleaves between nucleotide (nt) 31 and 32, producing termini with 5′-phosphate and 3′-OH. Next, the cleavage products were examined using total RNAs from HeLa cells. Only the small cleavage product was purified with sufficient quantity for MW determination, and it belongs to the first 31 nucleotides of human tRNA^{Phe}(FIG. 9A). Therefore, SAMD9¹⁵⁵⁻³⁸⁵ specifically cleaves tRNA^{Phe} amongst the numerous RNA in the human cells by targeting the same cleavage site as that of yeast tRNA^{Phe}, which is 5′ to the first nt of the anticodon loop.

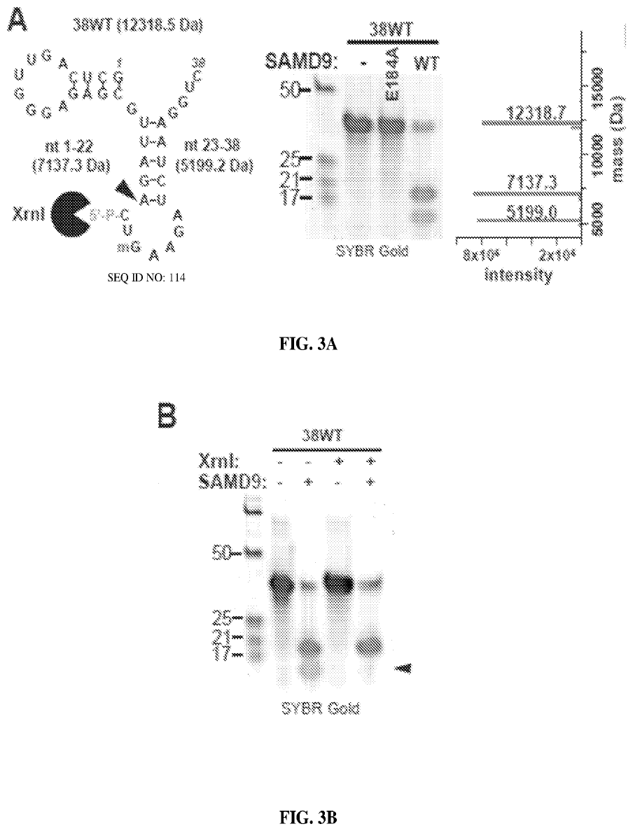

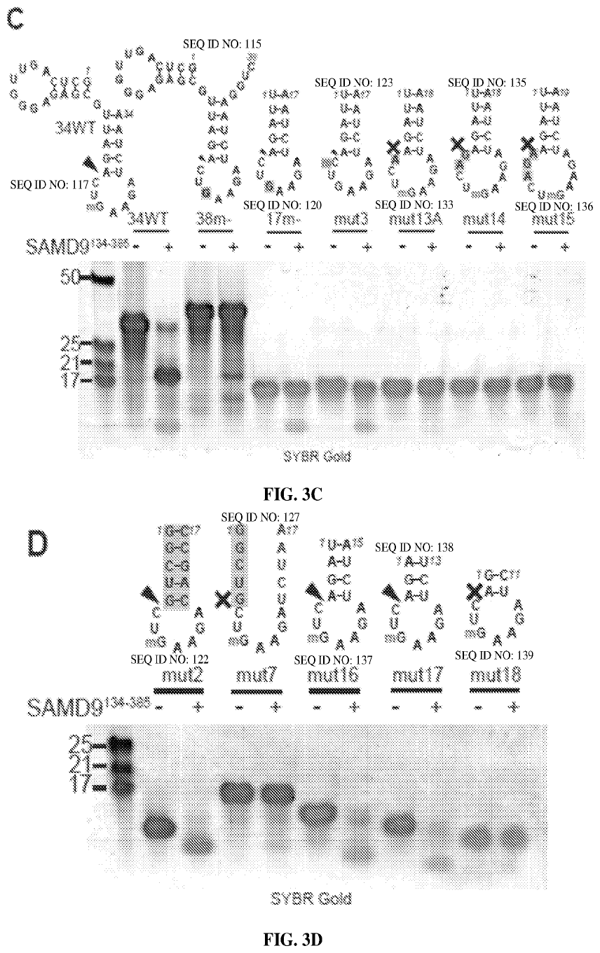

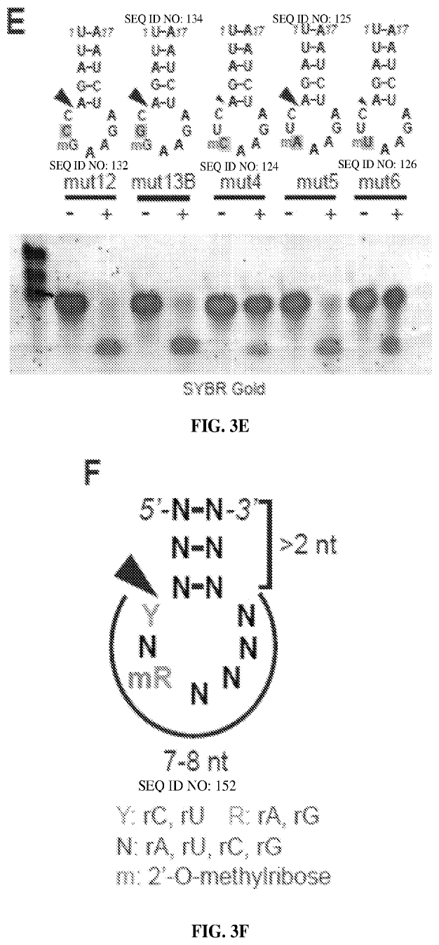

To determine the SAMD9¹³⁴⁻³⁸⁵ substrate specificity, regions of tRNA that are nearly identical between the human and yeast tRNA^{Phe} were the initial focus, which include the D arm and most of the anticodon arm (FIG. 9A). SAMD9¹³⁴⁻³⁸⁵ efficiently cleaved a 38-nt synthetic RNA, containing only the D arm, the anticodon arm, and the variable loop, at 5′ end of the anticodon loop (FIG. 3A). The 3′ cleavage product was readily degraded by exonuclease Xrn-1 (FIG. 3B), confirming that it has a 5′-P, which is required for Xrn-1 degradation. Deletion of the variable loop or mutation of the anticodon stem or the last two bases of the anticodon did not affect the cleavage efficiency or the cleavage site (FIG. 9B). Finally, a 17-nt synthetic RNA containing only the anticodon arm was found to be efficiently cleaved at 5′ end of the anticodon loop (FIG. 9C). Notably, these RNAs were all synthesized with 2′-O methylation at the wobble position of the anticodon but lack all other modifications in human tRNA^{Phe} (FIG. 9A).

The substrate specificity of SAMD9¹³⁴⁻³⁸⁵ was further assessed using additional synthetic variants of the 17-nt RNA. The absence of 2′-O methylation at the wobble position greatly reduced cleavage, even if the position corresponding to tRNA^{Phe} C32 was 2′-O methylated (FIG. 3C). A 17-nt RNA incapable of forming a stem was not cleaved, while a 13-nt RNA capable of forming just a 3-bp stem was cleaved (FIG. 3D). Substituting the wobble base with pyrimidines greatly reduced cleavage, while substituting its adjacent position with any bases had no negative impact (FIG. 3E). Unlike metal-independent RNases²⁵, 2′-OH at 5′ end of the scissile phosphate was not required for the cleavage but substituting ribose with deoxyribose in the stem or the loop abolished the cleavage (FIG. 9F). These and additional results collectively showed that SAMD9¹³⁴⁻³⁸⁵ cleaves a stem loop RNA with the feature shown in FIG. 3F) a loop of 7 or 8 nt and a stem of at least 3 base pairs, 2) a pyrimidine base at the first position of the loop, 3) a 2′-O-methylated purine base at the third position of the loop (corresponding to wobble position of the anticodon). The substrate specificity of SAMD9¹³⁴⁻³⁸⁵ can fully explain the exquisite specificity of SAMD9 on tRNA^{Phe} and the requirement of FTSJ1 for SAMD9 activity, as tRNA^{Phe} is the only tRNA that is known to have the 2′-O methylated purine at position 34. Other eukaryotic tRNAs known or suspected to be methylated by FTSJ1 at position 34, including tRNA^{TrP}(CC^{A}) and tRNAieu(C^{AA}), have pyrimidine base at the wobble position.

A slightly smaller fragment of SAMD9 that has its crystal structure solved, SAMD9¹⁵⁸⁻³⁶⁵ and its corresponding SAMD9L fragment, SAMD9L¹⁵⁸⁻³⁸⁸, were also capable of cleaving the natural tRNA^{Phe} as well as the 38-nt synthetic substrate (FIG. 9G-9I).

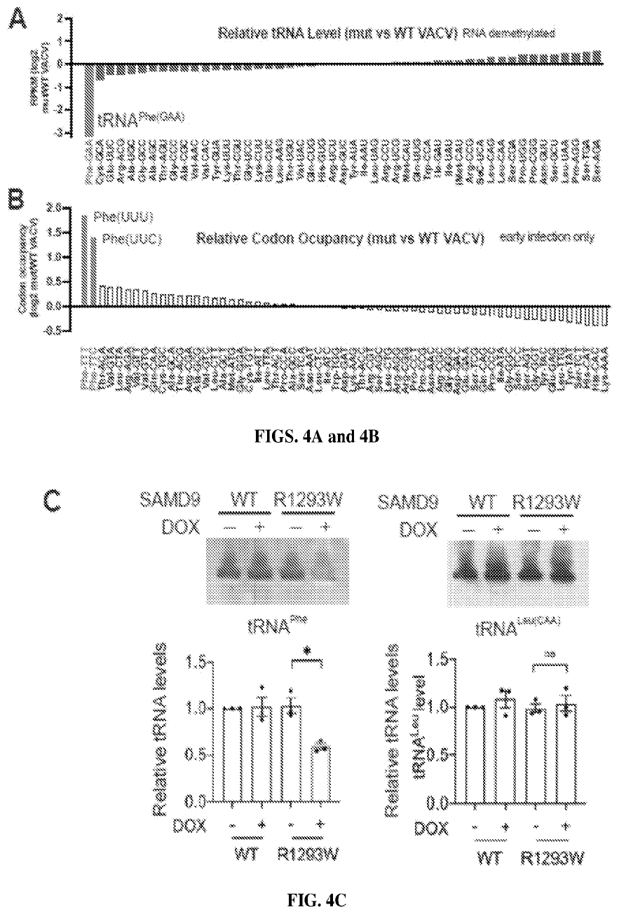

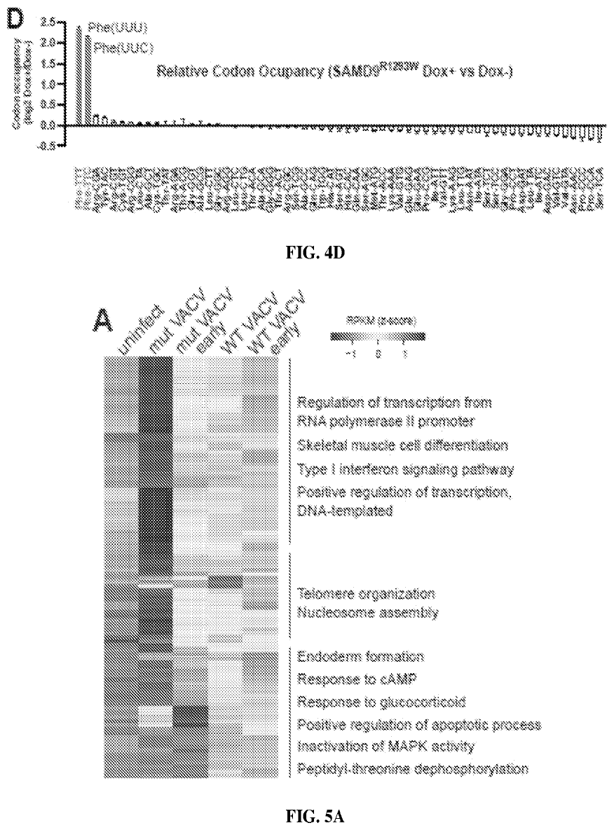

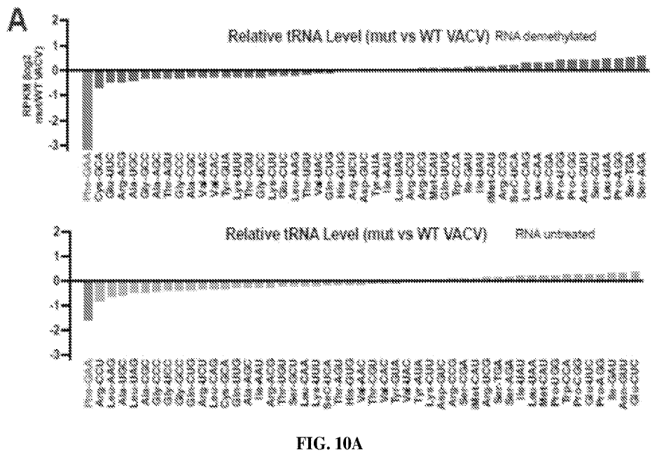

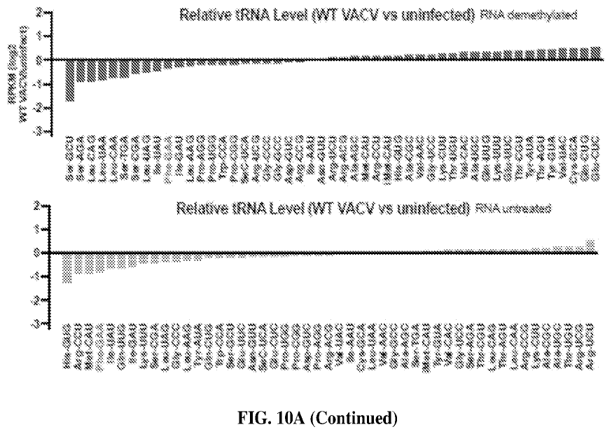

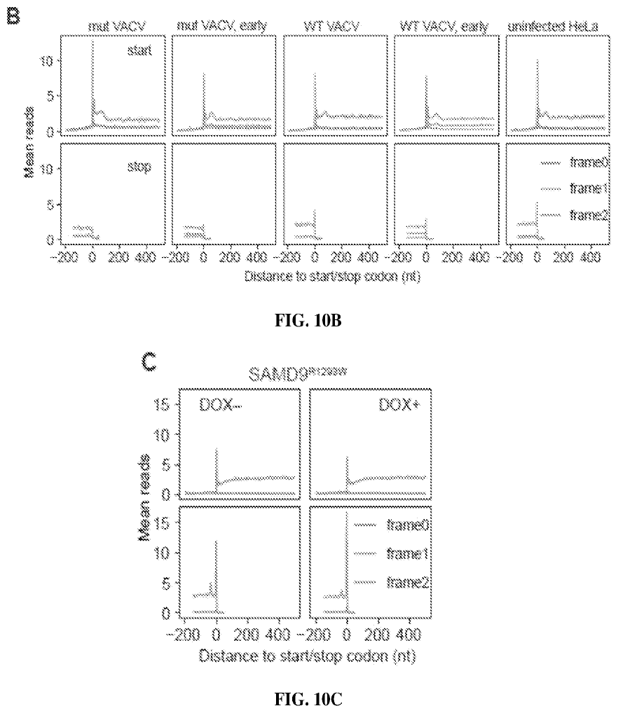

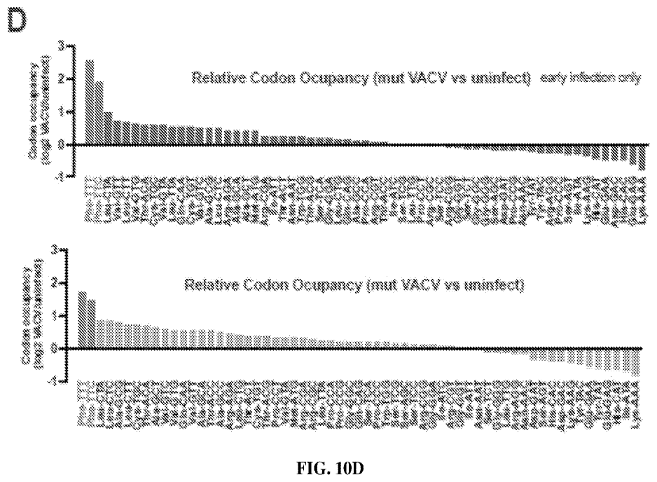

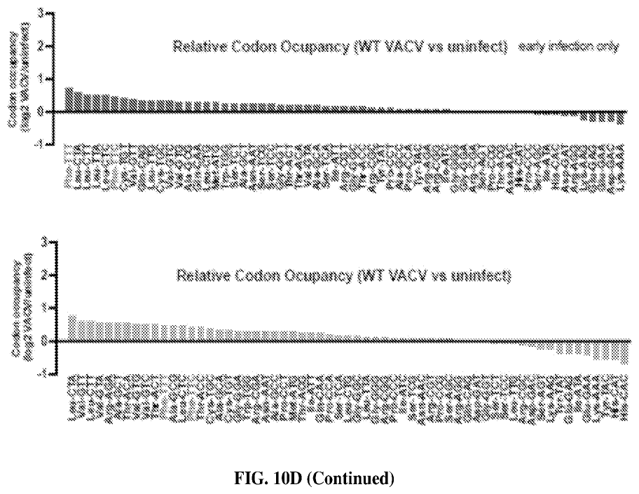

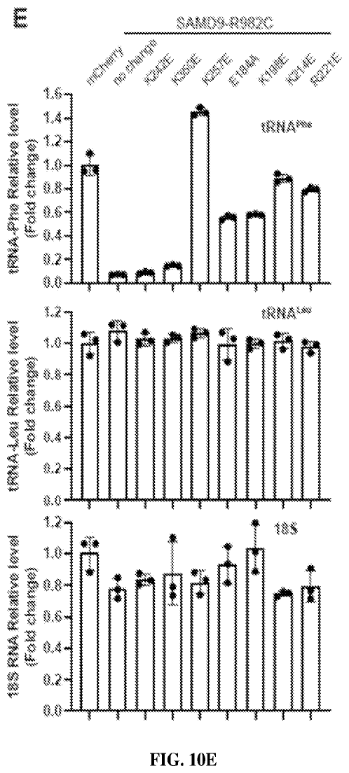

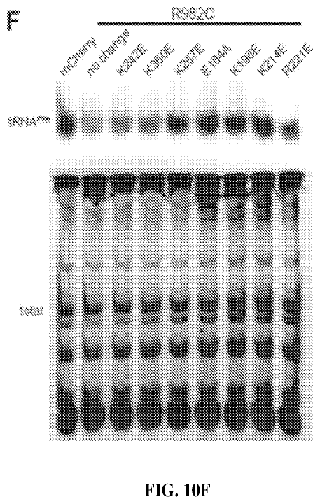

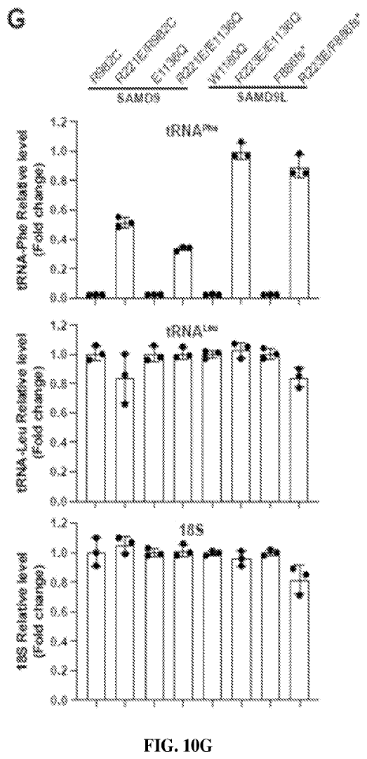

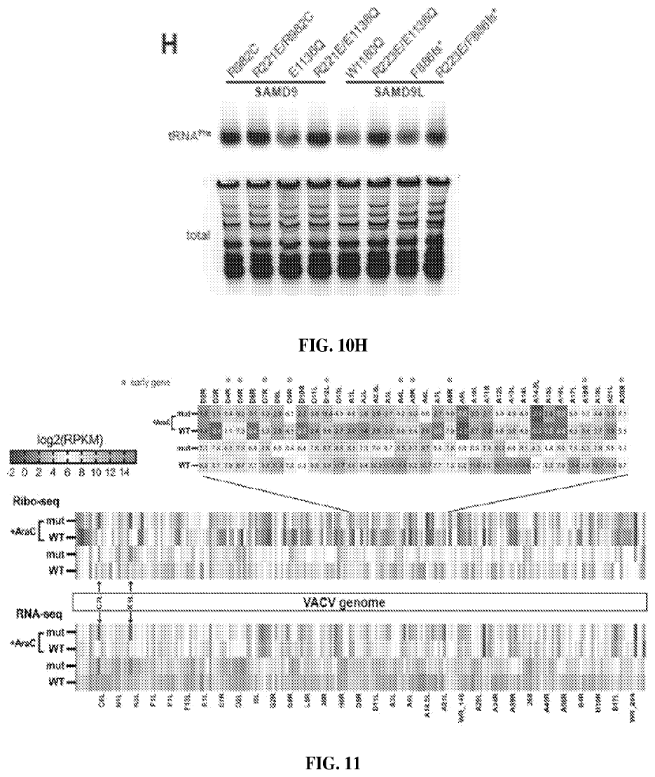

SAMD9 depletes cellular tRNA^{Phe} and causes codon-specific ribosomal pausing. To evaluate the effect of SAMD9 on intracellular tRNAs, a modified tRNA sequencing approach was used to quantify tRNA levels in cells infected with VACV. Relative to cells infected with WT VACV, vK1⁻C7⁻ infection drastically reduced the level of tRNA^{Phe} by nearly 10-fold, but not other tRNAs (FIG. 4A & FIG. 10A). Next, ribosome profiling (Ribo-seq) was conducted to determine the impact of tRNA^{Phe} depletion on global protein synthesis. Notably, cells infected with vK1⁻C7⁻ showed reduced ribosome occupancy in the coding region compared to cells infected with WT VACV or uninfected cells (FIG. 10B). With superior 3-nt periodicity of ribosome footprints, ribosome positions can be determined with high precision as evidenced by prominent peaks at start and stop codons (FIG. 10B). The A-site ribosome occupancy was calculated at individual codons by averaging the read density from the entire transcriptome. This analysis revealed a marked increase in the read density at two Phe codons UUU (~3.6-fold) and UUC (~2.6-fold) that are decoded by the single tRNA^{Phe} in cells infected with vK1⁻C7⁻, relative to that in cells infected with WT VACV or uninfected cells (FIG. 4B & FIG. 10D). To avoid the cascading effect of viral replication on cell physiology and to synchronize WT and vK1⁻C7⁻ VACV replication at the early stage, studies were repeated with virus infection in the presence of AraC to block viral DNA replication. Under this condition, the elevated read density at both Phe codons became more prominent in cells infected with vK1⁻C7⁻ (~5.8-fold for UUU and-3.6-fold for UUC) (FIG. 10D).

To substantiate this finding further, additional studies were performed on BT20 cells with Dox-inducible expression of WT SAMD9 or a gain of function (GoF) mutant SAMD9^{R1293W}. Without virus infection, the expression of SAMD9^{R1293W}, but not WT SAMD9, decreased cellular tRNA^{Phe} level by-50% (FIG. 4C). Importantly, the level of tRNA^{Leu(CAA)} remained comparable, supporting the specificity of tRNA targeting. Furthermore, testing of additional GoF SAMD9/9L variants in 293T cells, including R982C and E1136Q of SAMD9, and W1180R and F886Lfs*11 of SAMD9L, by transient transfection in 293T cells was performed. All the GoF variants tested were depleted of tRNA^{Phe} but had little effect on tRNA^{Leu(CAA)} (FIG. 10E-10H). A single substitution in the effector domain (R221E or R223E) that disrupted tRNA^{Phe} binding also prevented the GoF variants from depleting tRNA^{Phe} Mutations of other tRNA binding residues and the putative catalytic site also prevented SAMD9^{R982C} from depleting tRNA^{Phe}.

Ribo-seq analysis was performed on BT20 cells with Dox-inducible expression of SAMD9^{R1293W}. Remarkably, the two Phe codons UUU and UUC showed a drastic increase of read density at the A-site (~8.6- and 5.3-fold, respectively) upon Dox treatment (FIG. 4D). Similar to vK1⁻C7⁻ infection, the pausing at the UUU codon was greater than at the UUC codon, presumably because the relatively weaker binding affinity of the UUU codon with tRNA^{Phe}, due to the non-Watson-Crick U:G base-pairing at the wobble position, makes it more vulnerable to tRNA^{Phe} depletion.

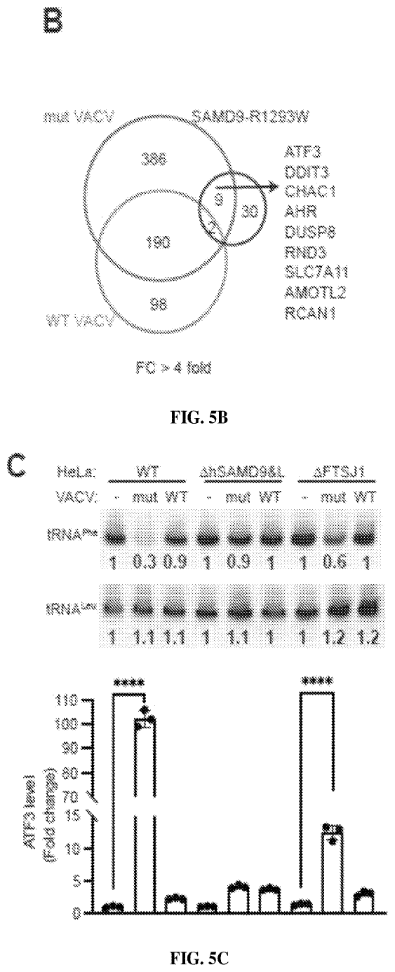

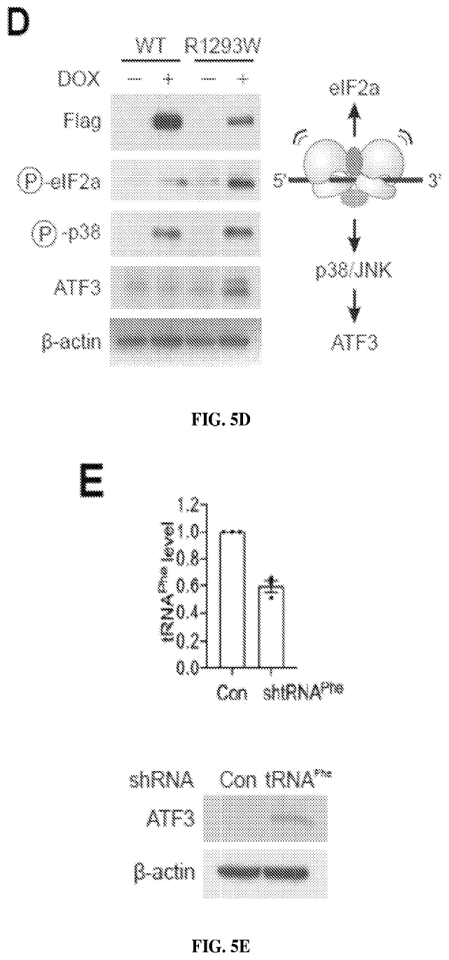

Activated SAMD9 triggers ribotoxic stress. To find out the common signaling cascade downstream of SAMD9 activation, transcriptome analysis was performed on the infected HeLa cells and compared to previous results from a GoF SAMD9 variant (FIGS. 5A-5B). SAMD9^{R1293W} was previously shown to significantly induce 41 genes that are mostly associated with proteotoxic stress response. Nine of 41 genes were also induced by more than 4-fold in response to infection by vK1⁻C7⁻ but not by WT VACV. Amongst the nine genes, ATF3 was induced the most with an increase of >60-fold. This was validated by RT-qPCR analysis, which additionally showed that the ATF3 induction in infected cells requires SAMD9 and partially requires FTSJ1 (FIG. 5C).

Persistent ribosome pausing is expected to cause ribosome collision and subsequent ribotoxic stress, which is characterized by increased eIF2α phosphorylation and ATF3 induction (FIG. 5D). Indeed, BT20 cells with the expression of SAMD9^{R1293W} induced ATF3 expression at mRNA and protein levels (FIG. 5D). Intriguingly, knocking down tRNA^{Phe} was sufficient to induce ribotoxic stress as evidenced by the upregulated ATF3 expression (FIG. 5E). Notably, silencing tRNA^{Leu} showed minimal effects on ATF3 induction, presumably due to its relative abundance.

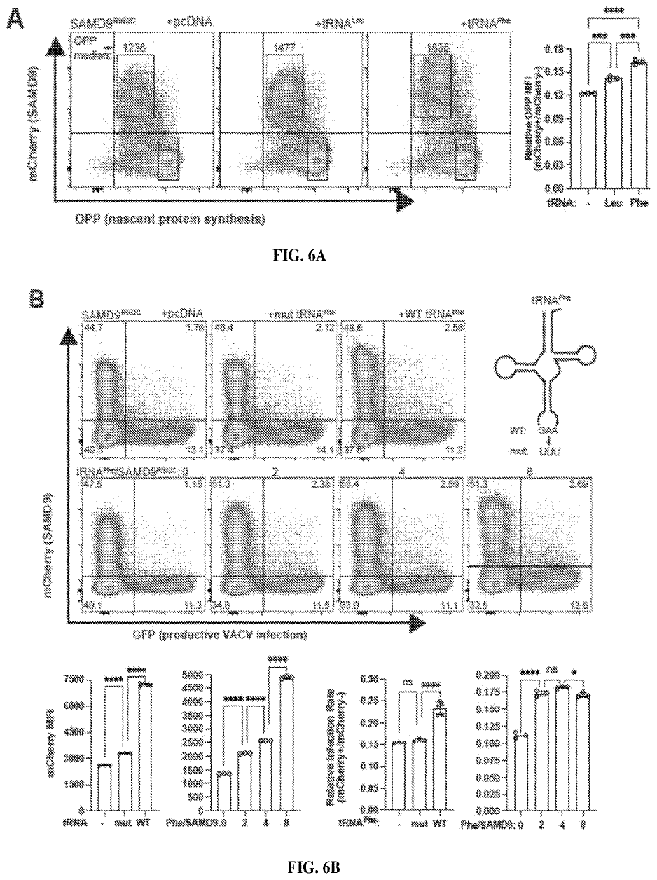

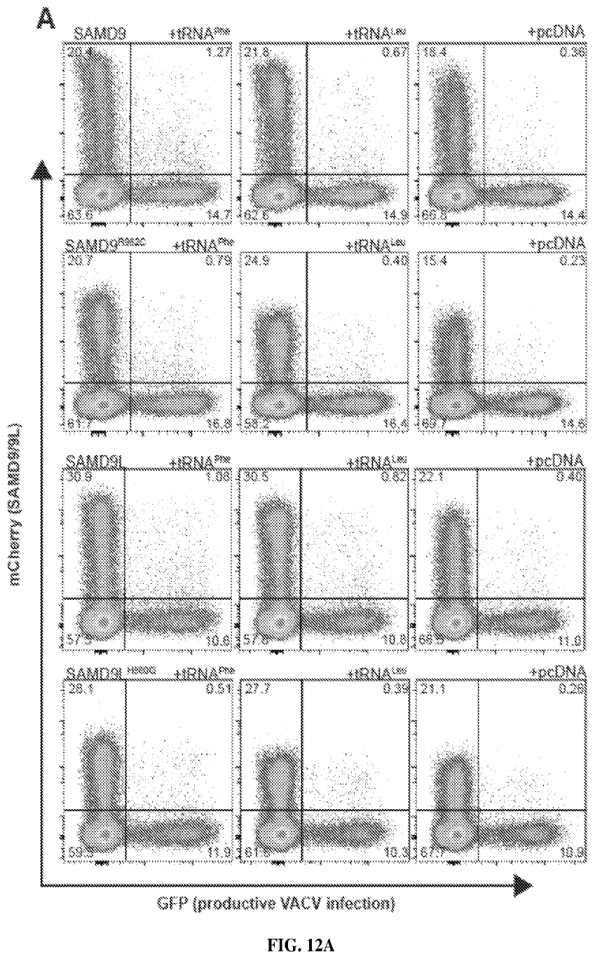

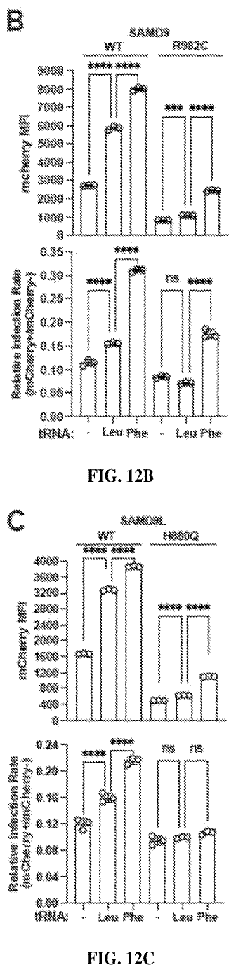

Overexpression of tRNA^{Phe} restores protein synthesis and viral replication. Next, alleviation of SAMD9/9L-mediated inhibition of protein synthesis and VACV replication was tested by ectopic overexpression of tRNA^{Phe}. To assess protein synthesis at single cell level, nascent cellular proteins were fluorescently labelled with a puromycin analog and performed flow cytometry analysis. As previously shown, transfection of mCherry-SAMD9^{R982C} fusion into 293T cells resulted in a strong inhibition of cellular protein synthesis in transfected (mCherry⁺) cells, relative to non-transfected cells (mCherry-) from the same culture well (FIG. 6A). Co-transfection of a plasmid overexpressing tRNA^{Phe} significantly increased cellular protein synthesis, including the synthesis of mCherry-SAMD9^{R982C} itself, in transfected cells. In comparison, co-transfection of a plasmid overexpressing tRNA^{Leu} had a minor effect. The effect of tRNA^{Phe} on cellular protein synthesis could also be detected from the total cell population by Western blot of puromycin-labeled proteins (FIG. 12).

To quantify the antiviral activity, 293T cells transfected with mCherry-SAMD9^{R982C} were infected with vK1⁻C7⁻ and analyzed by flow cytometry. The antiviral activity of SAMD9^{R982C} was reflected by ~10-fold reduction in infection rate in mCherry-SAMD9^{R982C}-expressing cells relative to that in non-transfected cells from the same culture well (FIG. 6B). Co-transfection of the tRNA^{Phe} plasmid increased the relative infection rate in transfected cells by-60%, even though the cellular level of mCherry-SAMD9^{R982C} was more than doubled by tRNA^{Phe} overexpression, indicating that the antiviral activity of SAMD9^{R982C} was attenuated. Importantly, a mutant tRNA^{Phe} with a 3-nt anticodon substitution had no effect on viral replication. tRNA^{Phe} overexpression increased the cellular synthesis of SAMD9^{R982C} proteins in a dose-dependent manner, but the increase in the relative infection rate was nearly constant, probably because further reduction in SAMD9 antiviral activity was counterbalanced by the increased SAMD9 protein level in the same cells. Finally, similar results were obtained using WT and additional GoF mutants of SAMD9/9L by showing that overexpression of tRNA^{Phe} restored protein synthesis and viral replication (FIG. 12C).

### DISCUSSION

SAMD9 and SAMD9L have emerged as important antiviral factors and myeloid tumor suppressors in recent years with the discoveries of SAMD9/9L as critical host restriction factors for poxviruses and GoF SAMD9/9L mutations as the cause for a spectrum of human diseases with myeloid involvement. They were predicted to function similarly to the STAND superfamily of signaling molecules, but their mechanisms of action had been elusive. Mammalian STAND proteins characterized to date are intracellular sensors utilizing their effector domains as an adaptor to recruit other cellular proteins. For example, the APAF1 effector is a CARD domain that recruits and activates caspase 1 to initiate apoptosis. In this example, the SAM D9 effector domain is an ACNase that has a direct effect on cellular protein synthesis and consequently cell proliferation and viral replication. Having an intrinsic effector function is unprecedented for mammalian STAND proteins but has been discovered recently in a family of bacterial antiviral STAND proteins, which contain N-terminal effectors with nuclease activities. This arrangement may allow the antiviral STAND proteins to respond to viral infection more rapidly.

The selected depletion of an essential tRNA by SAMD9 represents a previously unknown antiviral and antiproliferative mechanism in multicellular organisms. Analogous mechanisms are found in the microbial world, but the microbial proteins involved differ from SAMD9 in terms of structure and substrate specificity. Microbial ACNases characterized to date can be categorized into ribonucleases with BECR, HEPN or PIN domain. For example, PrrC contains a HEPN domain and cleaves tRNA^{lys(UUU)}; Colicin E5 contains a BECR domain and cleaves four different tRNA species; Enteric bacterial toxin VapC contains a PIN domain and cleaves tRNA^{fMET}. By contrast, SAMD9 ACNase has an Alba2 fold in its core and specifically target tRNA^{Phe}. Additionally, different post-transcriptional modifications at ASLs may influence the ACNase activities. PrrC activity is enhanced by the 6-threonylcarbamoyl adenosine modification at position 37, while the SAMD9 ACNase specificity is largely determined by the 2′-O-methylated purine base at the wobble position, which is present only in eukaryotic tRNA^{Phe} but not in other tRNAs or most of the prokaryotic tRNA^{Phe}. Notably, the 2′-O-methylation in tRNA^{Phe} is important for the organismal health. The lack of such modification causes slow growth for two diverse species of yeast and intellectual disability for humans. Steady state tRNA^{Phe} level was selectively reduced in the brain of FTSJ1 KO mice due to hypomodification, leading to decreased translation of a subset of the genes that are important for brain functions. Interestingly, GoF SAMD9L mutations are associated with ataxia-pancytopenia (ATXPC) syndrome characterized by cerebellar ataxia, showing that tRNA^{Phe} deficiency in the brain underlies the neurological manifestation of the disease.

Despite the difference in structure and specificity, SAMD9 and the microbial ACNases have the similar effects of depleting specific tRNAs and causing codon-specific inhibition of protein synthesis. This highlights the importance of tRNAs as regulators of protein synthesis and innate immunity in all domains of live. Some members of the mammalian Schlafen family also cleave tRNAs and serve as host restriction factors for several viruses, including the human immunodeficiency virus 1 (HIV-1). However, the Schlafen proteins cleave type II tRNAs 15 (tRNA^{Ser}) on the acceptor stem at the 3′-terminus and do not alter their steady state level. In the case of SAMD9/9L, tRNA^{Phe} deficiency has a major toxic effect associated with virus activated SAMD9 and GoF SAMD9/9L mutations and that overexpression of tRNA^{Phe} restores protein synthesis and virus replication, showing that increasing the steady state level of tRNA^{Phe} by overexpression or by inhibiting the ACNase activity is a therapeutic strategy for diseases associated with GoF SAMD9/9L mutations.

### Methods

Reagents. The following antibodies were used in the immunoblotting: anti-FTSJ1 (Santa Cruz, SC-390355), anti-Flag (Sigma, F1804), anti-ATF3 (Cell Signaling 18665S), anti-β-actin (Sigma A5441), anti-phosphorylated eIF2α (Cell signaling 3398P), anti-eIF2α (Cell signaling 5324), and anti-puromycin antibody (Developmental Studies Hybridoma Bank, PMY-2A4). Yeast Xrn-1 (NEB, M0338), yeast tRNA (Sigma, R4018), the microRNA marker (NEB, N2102S), and the Low Range ssRNA Ladder (NEB, N0364S) were acquired commercially.

Viruses. Wild type VACV WR strain and the K1L and C7L deletion mutant (vK1⁻C7⁻) were previously described. They were propagated and titrated on VERO cells.

Cells. VERO (ATCC CCL-81) and HeLa 229 (ATCC CCL-2.1) cells were from ATCC. HEK 293FT cells were from Thermo Fisher Scientific (cat. no. R70007). HeLa cells with SAMD9 and SAMD9L KO (ΔSAMD9&L) and BT cells with Dox-inducible expression of SAMD9-WT or SAMD9^{R1293W} were described before.

Plasmids construction. To overexpress tRNA^{Leu}(CAA) in vivo, the sequence including promoter region, tRNA sequence and terminator region were amplified by PCR using genomic DNA extracted from BT20 cells as template and cloned into EcoRI and PstI sites of pcDNA3.1. To overexpress tRNA^{Phe} in vivo, Q5 Site-Directed Mutagenesis Kit (NEB) was used to replace the tRNA^{Leu} sequence with tRNA^{Phe}. To knock out FTSJ1 gene, a pair of complementary oligonucleotides that contain the guide sequence for FTJS1 (5′-ATCTGTACCACACCTGGTAG-3′ (SEQ ID NO. 55)) was cloned into lentiCRISPRv2 (Addgene plasmid #52961) that had been digested with Esp3I, essentially as described.

Lentiviral plasmids for expressing FTSJ1 were constructed by PCR amplification of a FTSJ1 cDNA clone (Transomic Technologies) and subcloning the PCR fragment into NheI and BamHI sites of pCW57.1 (Addgene #41393), a doxycycline inducible lentiviral vector with rtTA-VP16-2A-puro. To decrease FTSJ1 basal expression level, the minimal CMV promoter upstream of the FTSJ1 gene was modified to match that in pTRE3G-IRES vector (Takara Bio) by using a synthetic DNA fragment (Eurofins). FTSJ1 K28A and A26P mutations were introduced into the plasmid through PCR with primers containing the desired mutations and subcloning the PCR fragments between NheI and EcoRI sites of the vector.

Plasmids for expressing mCherry-SAMD9/9L fusion (pcDNA6.2/mCherry-SAMD9/9L) were described previously. Specific mutations of SAMD9/9L were introduced into the plasmids through recombinant PCR based site-directed mutagenesis as described previously.

Generation of HeLa cell lines that express FTSJ1 allele under the Dox-inducible promoter. A FTSJ1 KO HeLa cell line was first generated by transient transfection of the plasmid containing the guide sequence for FTSJ1 to HeLa cells, following by selection with puromycin (3 μg/ml) for 2 days. Cells that survived the selection were cultured in the absence of puromycin, and multiple cell clones were isolated and tested for FTSJ1 expression by Western blotting. Cell clones that showed no FTSJ1 expression by Western blot were also tested for the FTSJ1 genotype by Sanger sequencing as described previously. The FTSJ1 KO cells were transduced with lentivirus encoding FTSJ1 WT or mutant alleles, similar as described previously. Transduced cells were selected with puromycin at 3 μg/ml.

Viral growth analysis. Cells in 12-well plates were incubated with 1 PFU per cell of the VACV for 2 h at room temperature. Following adsorption, the cells were washed twice with phosphate-buffered saline (PBS). One set of the cells was harvested immediately as the 0 hour post infection sample, while the other set were moved to 37° C. incubator to initiate viral entry and harvested at 24 hour post infection. The viral titers in the cell lysates were determined by plaque assays on VERO cells.

Protein purification. Recombinant SAMD9¹³⁴⁻³⁸⁵, SAMD9¹⁵⁶⁻³⁸⁵, and SAMD9L¹⁵⁸⁻³⁸⁸ proteins were expressed in E. coli as a SUMO fusion with a 6xHis-tag and purified as previously described.

Assay of SAMD9/9L activities in HEK 293T cells. HEK 293T cells in 24-well plates were transfected with pcDNA6.2/mCherry-SAMD9/9L plasmids before they were subjected to the following assays. For assessing antiviral activities, cells that had been transfected for 36 hours were infected with vK1⁻C7⁻/GFP⁺ at a MOI of 1 for 15 hours. For assessing protein synthesis level, cells that had been transfected for 24 hours were treated with 10 μM of O-propargyl-puromycin (OPP) for 30 min before they were processed with Click-iT Plus OPP Alexa Fluor 488 Protein Synthesis Assay Kit according to manufacturer's instruction (Thermo Fisher Scientific). The cells were harvested using Trypsin-EDTA Solution, fixed with 4% paraformaldehyde for 20 minutes, washed twice with Hanks' Balanced Salt Solution (HBSS) (Sigma Life Science) supplemented with 1% Bovine Serum Albumin (BSA), resuspended in 400 μL of HBSS, filtered with 40-micron mesh, and analyzed with an LSR-II cell analyzer (BD Biosciences). Flow data were analyzed using FlowJo software (TreeStar).

Protein synthesis rates in total transfected cells were also assessed by labeling the cells with 10 μg/ml of puromycin (InvivoGen) for 10 min followed by Western blot with an anti-puromycin antibody (PMY-2A4, Developmental Studies Hybridoma Bank).

EMSA. The binding of the purified SAMD9 proteins with yeast tRNA was studied by EMSA on a 0.8% native agarose gel as previously described.

In Vitro Cleavage Assay. 10 μg of total RNA extracted from HeLa cells or 20 pmol of synthetic RNAs was incubated with 0.8 to 14 μg of recombinant SAMD9¹³⁴⁻³⁸⁵ protein in the cleavage buffer at 37° C. for 60 min. The cleavage buffer was made of 40 mM Tris-HCl, pH 8.0, 20 mM KCl, 4 mM MgCl₂ (or MnCl₂) and 2 mM DTT. The cleavage products were mixed with gel loading dye (Invitrogen, 8546G) and resolved on 8% or 15% denaturing polyacrylamide gel containing 8 μM urea. The gels were stained with SYBR Gold (Invitrogen, S11494) or subjected to Northern blot analysis.

Northern blot Analysis. Northern blots were performed as described. Briefly, RNAs resolved on denaturing urea-polyacrylamide gel were transferred onto a positive charged nylon membrane. RNAs on the membrane were visualized and photographed after EtBr staining. The staining was washed away, and the RNAs on the membrane were crosslinked with short-wave UV light. The membrane was probed with digoxigenin-labeled DNA oligonucleotides against specific tRNAs. The labeling of oligonucleotides was performed with DIG oligonucleotide 3′-End labeling kit (Roche). The signal is detected with chemiluminescence with CDP-Star as the substrate (Roche).

Mass Spec. Oligonucleotide masses were determined with electrospray-ionization liquid chromatography mass spectroscopy by Novatia, LLC (Newtown, PA).

Immunoblotting. 12% SDS-PAGE gel was used to separate proteins with different mass and proteins were transferred to PVDF or nitrocellulose membranes (Fisher). Membranes were blocked in blocking buffer containing Tris-buffered saline (TBS), 5% nonfat milk, and 0.1% Tween-20 for 1 h at room temperature with shaking. Primary antibodies were diluted in blocking buffer (1:1,000) and added to membrane overnight at 4° C. After incubation with horseradish peroxidase-coupled secondary antibodies for 1 h at room temperature, the membrane was washed by TBST three times, and immunoblots were visualized using enhanced chemiluminescence (ECL-Plus, GE Healthcare).

Lentiviral shRNAs. shRNA sequence targeting tRNA^{Phe} (GAA) is 5′-TCAGTTGGGAGAGCGTTAGAC-3′ (SEQ ID NO. 56). shRNA sequence targeting tRNA^{Leu} (CAA) is 5′-TCTAAGGCGCCAGACTCAAGT-3′ (SEQ ID NO. 57). All the shRNA targeting sequences were cloned into DECIPHER pRSI9-U6-(sh)-UbiC-TagRFP-2A-Puro (Cellecta). Lenti-X 293T cells (Clontech) were used to package lentiviral particles. The supernatants containing virus were collected and filtered at 48 h after transfection. Then the lentivirus was added to cells for 24 h. After selection by 2 μg/μL puromycin, tRNA knockdown cells were collected and characterized.

qRT-PCR. For detecting mRNA levels, the complementary DNA (cDNA) was obtained by reverse transcription using a High-Capacity cDNA Reverse Transcription Kit (Invitrogen) or iScript Select cDNA Synthesis Kit (Bio-Rad). For detecting tRNA levels, the complementary DNA (cDNA) was obtained by rtStarrm tRNA-optimized First-Strand cDNA Synthesis Kit (ArrayStar). Real-time PCR was performed using Power SYBR Green PCR Master Mix (Applied Biosystems) and run on a LightCycler 480 Real-Time PCR System (Roche Applied Science) or CFX96 Real-Time system (Bio-Rad). Relative expression was calculated using the 2^{-ΔΔCt} method.

tRNA-seq. Total RNA was deacylated in 50 mM Tris-HCl (pH 9.0) at 37° C. for 45 min and then resolved by 10% polyacrylamide TBE-urea gel (Invitrogen). tRNA (60 to 100 nt) were collected by gel excision and resolved in RNA elution buffer (300 mM NaOAc pH 5.2, 1 mM EDTA, 0.1 U/μL SUPERase In) overnight. After ethanol precipitation, tRNA was treated with demethylase (rtStar™ tRNA-optimized First-Strand cDNA Synthesis Kit, ArrayStar) and cleaned up. 1 μg tRNA was used for library construction. Briefly, tRNA was dephosphorylation in the 15 μL mixture containing 1×T4 polynucleotide kinase buffer, I U/μL SUPERase In, and 1 U/μL T4 polynucleotide kinase at 37° C. for 30 min and followed by 65° C. for 20 min. After ethanol precipitation, RNA was pelleted by centrifugation for 15 min at 20,000g 4° C. and dissolved in nuclease free H₂O. Poly(A) tailing and 5′ adenylation reaction was carried in the mixture containing 1×Poly(A) tailing reaction buffer, 1 mM ATP, 1 U/μL SUPERase In, 1 U/μL Poly(A) polymerase and 1 U/μL Ezra at 37° C. for 30 min and followed by 65° C. for 20 min. RNA sample was then mixed with 1 μL 1 μM ligation adaptor (5′-AATGATACGGCGACCACCGAGATCTACACTCTTTCCCTACACGACGCTCTTCCGA TCT rGrGrG-3′ (SEQ ID NO. 58)) and heated at 65° C. for 2 min, then immediately put on ice for 1 min. Ligation was performed in a 5 μL mixture containing 1×T4 RNL2 reaction buffer, 1 U/μL SUPERase In, 15% PEG8000, and 20 U/μL T4 RNA ligase 2 truncated at 22° C. for 1.5 h. For reverse transcription reaction, 1 μL 1 μM RT primer (5′-GCATCTCGGTGGTCCAATCTGAACTCCAGTCGTGTGACTGGAGTTCAGACGTGTG CTCTTCCGATCTTTTTTTTTTTTTVN-3′ (SEQ ID NO. 59)) was added to the ligation products and denatured at 65° C. for 2 min and cooled down on ice for 1 min. The linker ligated RNA was then mixed with 1×first strand buffer, 0.5 mM dNTP, 5 mM DTT, 2 U/μL RNaseOUT, and 200 U/μL SuperScript IV at 50° C. for 1 h, followed by 80° C. for 10 min. PCR was performed in a 20 μL mixture containing 1×HF buffer, 0.2 mM dNTP, 0.5 μM forward PCR primer (5′-AATGATACGGCGACCACCGAGATCTACACTCTTTCCCTACACGACGCTCT-3′ (SEQ ID NO. 60)), 0.5 μM reverse PCR primer with barcode (See below) and 0.5 U Phusion polymerase. The PCR was initiated at 98° C. for 30s, then 98° C. for 5s, 68° C. for 20s, 72° C. for 15 s for 12 cycles. PCR products were separated on a nondenaturing 8% polyacrylamide TBE gel. Expected DNA at 200-240 bp was excised and soaked by 400 μL DNA elution buffer (300 mM NaCl, 1 mM EDTA) overnight. After removal of gel debris using Spin-X column, samples were ethanol precipitated and dissolved in 15 μL nuclease-free H₂O. Finally, samples were sequenced (Illumina HiSeq) by using sequencing primer.

library-PCR-R-13:
(SEQ ID NO. 61)
5′-CAAGCAGAAGACGGCATACGAGAT TGTTGACT
GTGACTGGAGTTCAGACGTGTGCTCT-3′
library-PCR-R-14:
(SEQ ID NO. 62)
5′-CAAGCAGAAGACGGCATACGAGAT ACGGAACT
GTGACTGGAGTTCAGACGTGTGCTCT-3′
library-PCR-R-15:
(SEQ ID NO. 63)
5′-CAAGCAGAAGACGGCATACGAGAT TCTGACAT
GTGACTGGAGTTCAGACGTGTGCTCT-3′
library-PCR-R-16:
(SEQ ID NO. 64)
5′-CAAGCAGAAGACGGCATACGAGAT CGGGACGG
GTGACTGGAGTTCAGACGTGTGCTCT-3′
library-PCR-R-18:
(SEQ ID NO. 65)
5′-CAAGCAGAAGACGGCATACGAGAT GTGCGGAC
GTGACTGGAGTTCAGACGTGTGCTCT-3′
library-PCR-R-19:
(SEQ ID NO. 66)
5′-CAAGCAGAAGACGGCATACGAGAT CGTTTCAC
GTGACTGGAGTTCAGACGTGTGCTCT-3′
library-PCR-R-20:
(SEQ ID NO. 67)
5′-CAAGCAGAAGACGGCATACGAGAT AAGGCCAC
GTGACTGGAGTTCAGACGTGTGCTCT-3′
library-PCR-R-21:
(SEQ ID NO. 68)
5′-CAAGCAGAAGACGGCATACGAGAT TCCGAAAC
GTGACTGGAGTTCAGACGTGTGCTCT-3′

Ribo-seq. Ribo-seq in BT20 cells was performed as follows. Ribosome fractions separated by sucrose gradient sedimentation were pooled and digested with E. coli RNase I (Ambion, 750 U per 100 A260 units) by incubation at 4° C. for 1 h. SUPERase inhibitor (50 U per 100 U RNase I) was then added into the reaction mixture to stop the digestion. Total RNA was extracted using TRIzol reagent. RNA was separated on a 15% polyacrylamide TBE-urea gel (Invitrogen) and the ribosome protected fractions were excised (25-35 nt). After gel soaking and RNA precipitation, RNA was collected for library construction as described above. Finally, expected DNA at 160-180 bp was excised and collected for sequencing.

Ribo-seq in VACV infected cells was performed essentially as described. The steps include RNase I treatment, ribosome recovery, footprint fragment purification, 3′adaptor ligation, rRNA depletion, reverse transcription, circularization, and PCR amplification.

Alignment of sequencing reads. The 3′ adapter was trimmed by Cutadapt. Trimmed reads with length <15 nucleotides were excluded. For Ribo-seq and RNA-seq, the remaining reads were mapped to a house-hold database including all putative human rRNAs by using Bowtie. Mismatch is not permitted. The reads that cannot be aligned to rRNAs were aligned to human transcriptome using STAR. To construct the transcriptome, the annotation file from the ENSEMBL database (GRCh38) was used. For each gene, the mRNA with the longest CDS was selected. In the case of equal CDS length, the longest transcript was used. For read alignment, a maximum of two mismatches were permitted. To avoid ambiguity, reads that were mapped to multiple positions were excluded. For tRNA-seq, the adaptor trimmed reads were aligned to human genome (GRCh38) using HISAT2, the reads that were aligned to the human tRNAs were extracted, and then were aligned to a household tRNA database that contains a unique tRNA sequence for each type of tRNAs. Mismatch is not permitted. For the reads that can be aligned to multiple tRNAs, the read is counted as 1/n for each tRNA (n is the total number of tRNAs that can be aligned).

Aggregation plot of footprint reads near the start and stop codons. The ribosome P-site was defined as positions +12, +13, and +14 from the 5′-end of the reads (the first position of the reads is recorded as 0). For each mRNA, the mRNA with total reads in the CDS <32 was excluded from analysis. Ribosome densities at individual mRNA sites were counted and then normalized by the average ribosome density of the CDS. Ribosome densities with the same distance from the start or stop codon were averaged over the whole transcriptome.

Estimation of ribosome density on mRNAs and mRNA levels. For each mRNA, a RPKM (Reads Per Kilobase of transcript, per Million mapped reads) value was calculated, and used to measure the relative ribosome density on individual mRNAs. For Ribo-seq, only the reads aligned to the CDS were used. For RNA-seq, the reads aligned to mRNAs were used. mRNAs with RPKM values <1 were excluded.

Calculation of ribosome occupancy. For each mRNA, ribosome densities at individual codons of the CDS were normalized to the average density of the CDS. Second, ribosome occupancies at the same codons were averaged over the whole transcriptome.

Quantification and statistical analysis. Data were analyzed using GraphPad Prism 9 software.

### Example 3. Inhibitor Discovery for the Human SAMD9 tRNA Endoribonuclease

Mutations in human SAMD9 and SAMD9L (SAMD9/9L) genes cause life-threatening diseases with limited treatment options. Recent studies have revealed SAMD9/9L as latent endoribonucleases targeting phenylalanine tRNA (tRNA-Phe) and that their activities are constitutively activated by pathogenic human mutations. Herein, a continuous fluorophore-based enzymatic assay for human SAMD9 tRNase was developed, exhibiting high sensitivity and reproducibility. The assay enabled an examination of SAMD9 tRNase enzyme kinetics. Furthermore, a high-throughput screen of over 32k small molecules for SAMD9 tRNase inhibitors identified over 40 leads, illustrating the feasibility of small molecule inhibitors targeting the pathogenic effects of SAMD9/9L mutations. Evans Blue and Salvianolic acid B were further validated through additional assays as inhibitors disrupting SAMD9 tRNase binding and cleavage of tRNA-Phe. These inhibitors serve as tools for exploring the mechanism of action of small molecule inhibitors and as starting points for developing drugs with enhanced efficacy and specificity.

The paralogous genes SAMD9 and SAMD9L (SAMD9/9L), located on human chromosome 7, encode two cytoplasmic proteins with over 60% amino acid sequence identity. Functioning as cell-intrinsic antiviral factors, SAMD9/9L proteins are critical restriction factors for poxviruses. Beyond antiviral responses, they play an important but incompletely understood role in myeloid homeostasis. Since 2016, reports have revealed SAMD9/9L mutations as the underlying causes for several life-threatening human diseases involving the myeloid tissues. Germline SAMD9 mutations were first linked to MIRAGE syndrome, a multisystem developmental disorder with myelodysplasia, infection, restriction of growth, adrenal hypoplasia, genital phenotypes, and enteropathy. In parallel, SAMD9L mutations were first linked to ataxia-pancytopenia syndrome, characterized by a progressive neurological phenotype, cytopenia and bone marrow hypoplasia. Subsequent investigations identified SAMD9/9L mutations as a major cause of inherited bone marrow failure (IBMF) syndromes and pediatric myelodysplastic syndromes (MDS), contributing to 8% to 18% of cases in different cohorts. Additionally, frameshift mutations that truncate SAMD9L within a specific region were found to cause SAMD9L-associated autoinflammatory disorder (SAAD) characterized by severe inflammation and B cell deficiency.

These diverse disorders are associated with heterozygous SAMD9/9L mutations that confer a gain-of-function (GoF) of suppressing cell growth in vitro, frequently leading to monosomy 7 in hematopoietic cells. The loss of the chromosome 7 that carries the growth-suppressive SAMD9/9L mutations is a somatic genetic rescue (SGR) mechanism, escaping the hematotoxic effects of SAMD9/9L mutations. Monosomy 7, which predisposes cells to acquiring somatic cancer mutations, has long been associated with myeloid malignancies. To date, hematopoietic stem cell transplantation (HSCT) represents the only reported therapy for treating SAMD9/9L disorders, albeit with limited success.

Cell growth inhibition is the major pathogenic effect of patient-derived SAMD9/9L mutations, but its underlying molecular mechanism has not been completely defined. Earlier studies indicated that SAMD9/9L affect endosomal fusion and growth factor recycling. Recent studies revealed that GoF SAMD9/9L variants induce a global translational repression in both cell lines and hematopoietic progenitor cells. Specifically, SAMD9 has been identified as a poxvirus-activatable endoribonuclease, depleting the essential tRNA-phenylalanine (tRNA-Phe). This depletion leads to ribosomal pausing at phenylalanine codons and triggers stress responses. While the tRNase activity of SAMD9 is latent in uninfected cells, missense mutations identified in human patients render it constitutively active, resulting in a global translational block. Moreover, a domain within the multi-domain SAMD9 protein has been identified as a metal-dependent endoribonuclease that specifically cleave tRNA-Phe. SAMD9 tRNase has a unique substrate specificity for a stem loop RNA with a strategically located 2′-0 methylated purine, which is uniquely found in tRNA-Phe. The structure of SAMD9 tRNase also appears to be distinct from previously characterized endoribonuclease.

The identification of SAMD9 as a new tRNA endoribonuclease provides a molecular target for developing therapies for patients affected by GoF SAMD9/9L mutations. Herein, a quantitative fluorescence assay was established to characterize the enzymatic activities of SAMD9 tRNase and screen for inhibitors. A high throughput screening of over 32 thousand compounds yielded over 40 leads, demonstrating the feasibility of small molecule inhibitors targeting the pathogenic effects of patient-derived SAMD9/9L mutations.

### Methods

Protein and RNA. The recombinant SAMD9¹³⁴⁻³⁸⁵ or SAMD9¹⁵⁶⁻³⁸⁵ protein was expressed and purified from E. coli. In brief, SAMD9¹³⁴⁻³⁸⁵ or SAMD9¹⁵⁶⁻³⁸⁵ fused with SUMO and a 6xHis-tag was purified from soluble cell lysate using a Ni-NTA affinity column. Subsequently, the fusion protein underwent Ulp1 protease cleavage and passed through another Ni-NTA column. The resulting flow-through was collected and subjected to size exclusion chromatography for final purification.

6-Fam-labeled oligonucleotides were synthesized by Integrated DNA Technologies (Coralville, IA) and underwent RNase-Free HPLC purification. The sequence of Phe17-Fret is /56-FAM/UUAGACUmGAAGAUCUAA/3IABkFQ/(SEQ ID NO: 156), where 56-FAM indicates 6-carboxyfluorescein (FAM)-labeling at the 5′ end, 3IABkFQ indicates Iowa Black FQ quencher labeling at the 3′ end, and mG indicates guanidine with a ribose 2′-O methylation. The sequence of Phe17-Fret-noM is/56-FAM/UUAGACUGAAGAUCUAA/3IABkFQ/(SEQ ID NO: 157).

FRET-based in vitro cleavage assay. A 10 μl reaction was set up in a 384-well plate (Corning, plate #4514) at room temperature. The reaction included 1× cleavage buffer (40 mM Tris-HCl, pH 8.0, 20 mM KCl, 4 mM MnCl₂ and 2 mM DTT) and various concentrations of SAMD9¹³⁴⁻³⁸⁵ protein and Phe17-Fret. Phe17-Fret was added last and immediately before reading the fluorescence intensity every two minutes on a microplate reader (BioTek Synergy 2 or BMG Pherastar FS) with excitation at 485 nm and emission at 520 nm.

High-throughput screening of inhibitors. Around 32,000 compounds were screened, including 9,600 compound from ChemBridge NOVACore library, 3,500 compounds from Life Chemicals Covalent library and FSP3 library, 12,800 compounds from Life Chemicals Diverset library, and 5,000 compounds from TargetMol FDA approved and Bioactive library. For screening, the final concentration for the compounds was 15 μM for NOVACore, FSP3 and Diverset, and 10 μM for TargetMol and Covalent. The screening condition was set up as follows at room temperature. 0.5 μl of the compounds from various libraries was dispensed into a 384-well assay plate (Corning, plate #4514) using an Echo liquid handler (Beckman Coulter), followed by the addition of 10 μl of SAMD9¹³⁴⁻³¹⁵ protein at 600 nM with EL406 (BioTek). After incubating for 20-30 min, 5 μl of Phe117-FRET substrate at 1500 nM was dispensed into the well. After another hour of incubation, fluorescence reading was taken by the Pherastar FS plate reader (BMG Labtech). The final reaction volume was 15 μl containing 4 mM MnCl₂, 0 (for covalent library compounds) or 0.5 mM (for other compounds) DTT, 400 nM SAMD9¹³⁴⁻³⁸⁵ protein, and 500 nM phe17-FRET. Compounds that reduced the fluorescence signal by 50% were considered hits and selected for dose-dependent studies, in which 10 3-fold diluted concentration series were tested. The dose-dependent studies were done as described above in a total reaction volume of 10 μl and compound concentrations ranging from 50,000 nM to 2.53 nM. The half-maximal inhibitory concentration (IC₅₀) of each compound was calculated using GraphPad Prism software.

Gel-based cleavage assay. For gel-based assays, the following compounds were purchased from MedChemExpress (Monmouth Junction, NJ): Pyridostatin hydrochloride (HY-15176A), Salvianolic acid B (HY-N1362), and Evans Blue (HY-B1102). The compound was incubated with 220 nM of recombinant SAMD9¹³⁴⁻³⁸⁵ protein for 20 mins before 2 μM of the yeast tRNA^{Phe} (Sigma-Aldrich, R4018) was added. The concentrations for Pyridostatin hydrochloride, Salvianolic acid B, and Evans Blue were 15.6 μM, 6 μM, and 2.4 M, respectively, which are approximately 2×IC50 obtained from the high-throughput screening. After 2 hours of reaction at room temperature, the reaction was stopped by adding the gel loading buffer (Invitrogen, 8546G) and resolving on denaturing polyacrylamide gel containing 8 μM urea. The microRNA marker (NEB, N2102S) and the Low Range ssRNA Ladder (NEB, N0364S) were used as the molecule weight markers. The gels were stained with SYBR Gold (Invitrogen, S11494).

EMSA. The abilities of the compounds to compete with tRNA for binding to SAMD9¹³⁴⁻³⁸⁵ were studied by EMSA on a 0.8% native agarose gel. In brief, purified recombinant SAMD9¹³⁴⁻³⁸⁵ protein (8 M) was mixed with an equal molar of yeast tRNA^{Phe} (Sigma-Aldrich, R4018), and various concentrations of the compound in a total volume of 25 μl. After incubation on ice for 1 hour, the mixtures were analyzed on a 0.8% native agarose gel with 0.05 μg/ml ethidium bromide (EtBr) in 1×Tris-EDTA (TE) buffer under 90V for 60 mins at 4° C. A UVP benchtop UV transilluminator was used for the visualization of the EtBr-stained tRNA. The same gel stained with Coomassie blue was visualized on a Canon CanoScan LIDE 100 scanner.

### Results

### FRET-Based Cleavage Assay.

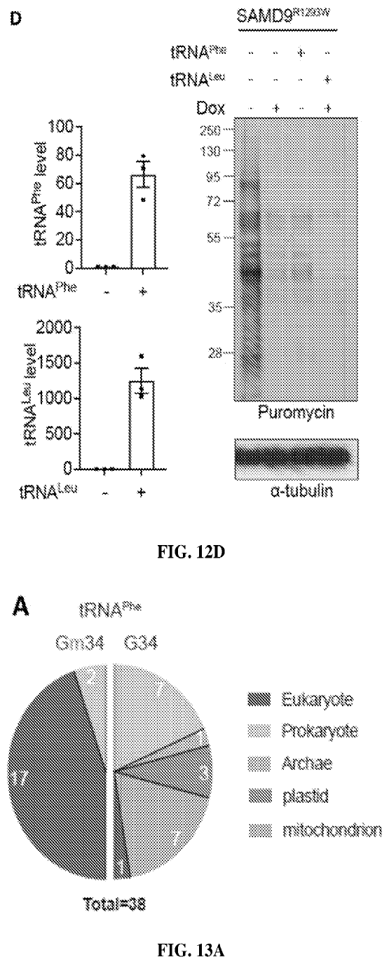

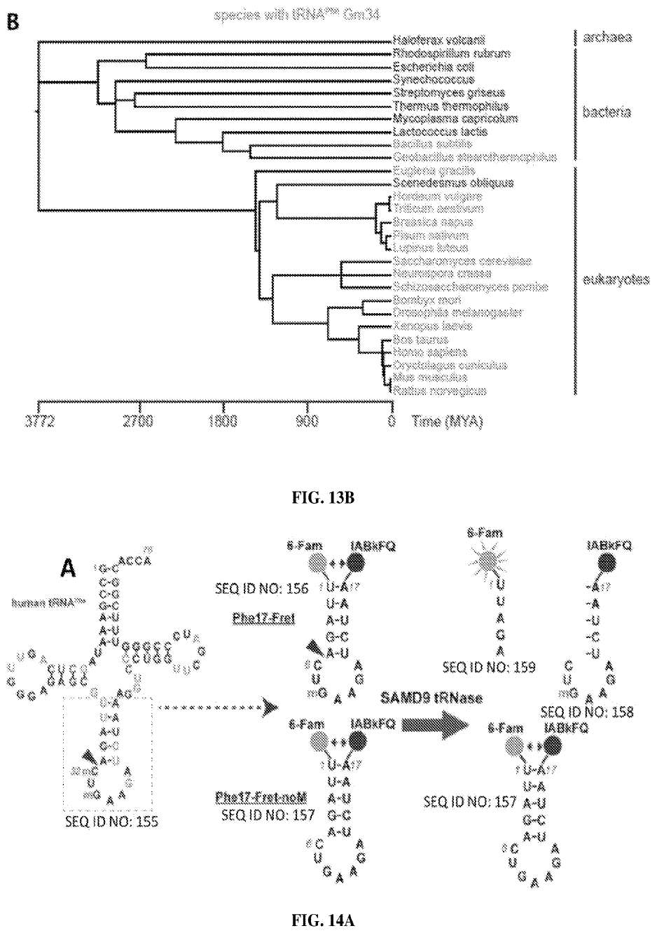

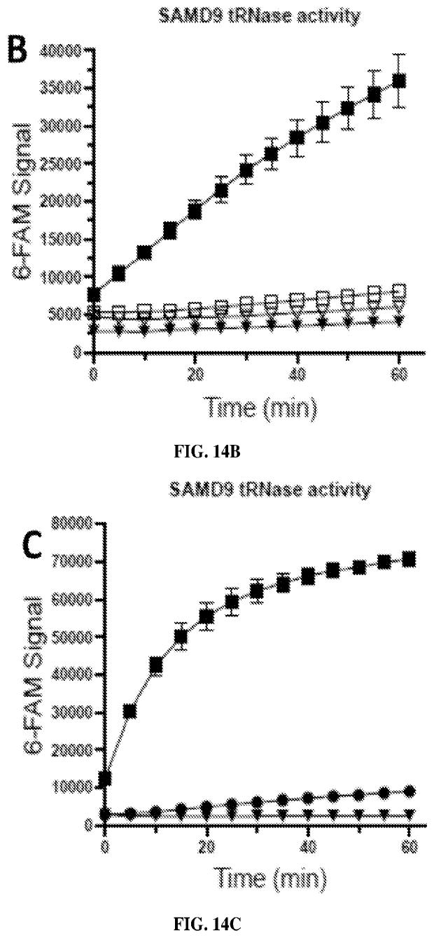

Previously a gel-based assay was used, SAMD9 tRNase was found to specifically cleave a 17-nt synthetic RNA mimicking the anticodon stem loop of human tRNA-Phe. Key features of the RNA that make it a specific substrate include its stem-loop structure, a pyrimidine base at the loop's first position, and a 2′-O-methylated purine base at the wobble position of the anticodon. To continuously monitor the cleavage reaction in real time, another RNA substrate named Phe17-Fret was synthesized, adding to the 17-nt RNA a 6-FAM fluorescent doner dye to its 5′ end and a quencher (IBFQ) to its 3′ end (FIG. 14A). Another RNA, differs from Phe17-Fret only by the omission of 2-O-methylation at the wobble base, was synthesized as a negative control substrate. Both synthetic RNAs exhibited minimal fluorescence due to the close proximity of 3′-IBFQ to 5′-6-FAM in the stem loop, quenching the 6-FAM fluorescence via Förster resonance energy transfer (FRET). Upon adding SAMD9 tRNase to Phe17-Fret, a steady increase in 6-FAM fluorescence occurred (FIG. 14B). In contrast, no change was observed with the non-2′-O-methylated RNA. Moreover, the addition of SAMD9 tRNase with the inactivating E184A substitution to Phe17-Fret resulted in minimal fluorescence (FIG. 14C). These data confirm that the fluorescence signal arises from the cleavage of Phe17-Fret by SAMD9 tRNase, separating the 5′ 6-FAM from the 3′ quencher.

SAMD9 tRNase Enzyme Kinetics.

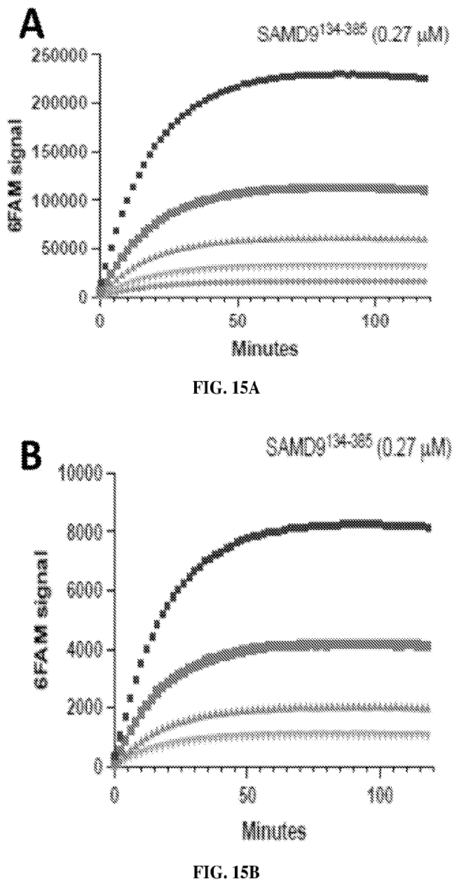

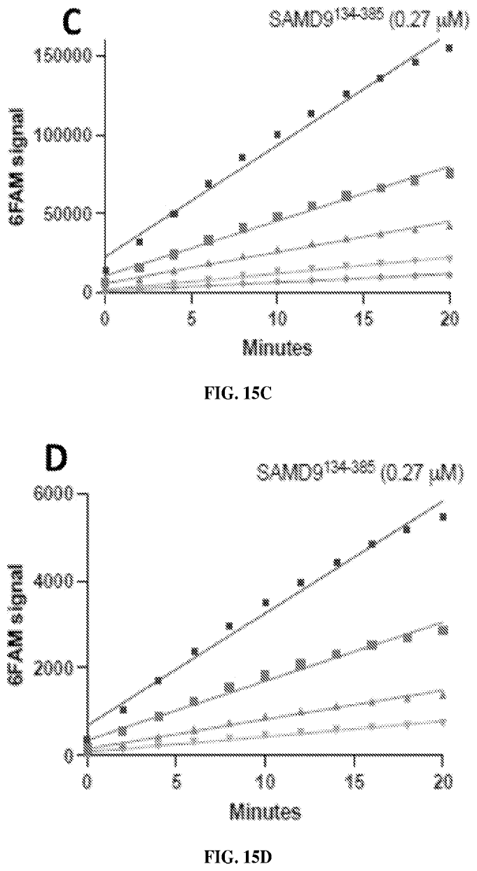

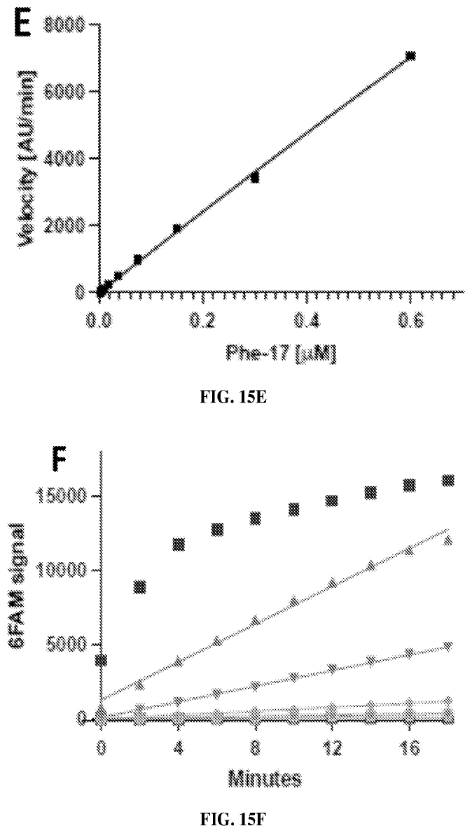

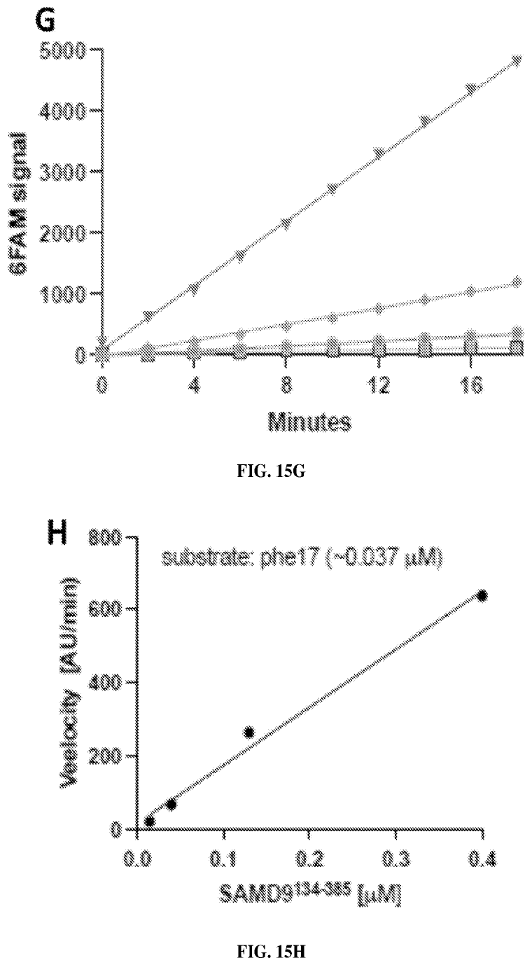

Using the FRET-based cleavage assay, the enzyme kinetics of SAMD9 tRNase was studied (FIGS. 15A, 15B, 15C, 15D, 15E, 15F, 15G, and 15H). In one setting, the enzyme concentrations were varied, while fixing the Phe17-Fret substrate concentration at 0.037 μM. In another setting, the substrate concentrations were varied, while fixing the SAMD9 tRNase enzyme concentration at 0.27 M. With enzyme:substrate ratios from 1:100 to 10:1, the fluorescence signal increased with a steady velocity within the first 20 minutes and approached plateau after 1 hour. Initial velocities during the first 20 minutes were in linear proportion to the enzyme and substrate concentrations.

High-Throughput Screening of Small-Molecule Inhibitors of SAMD9 tRNase.

Additional tests of the Fret-based cleavage assay were performed to optimize the condition for high-throughput inhibitor screening. The tests showed that the fluorescence signal saturated with Phe17-Fret substrate concentration at 800 nM and that the fluorescence signal remained strong for at least 4-5 hours. Using 400 nM enzyme concentration, 500 nM substrate concentration, and a one-hour reaction time, around 32,000 small molecules at a concentration of either 10 or 15 μM were screened. Employing a cutoff of more than 50% reduction in fluorescence signal, 9 hits were identified from the TargetMol FDA approved and Bioactive library, while 34 hits were identified from Life Chemicals Covalent libraries. All hits underwent dose-response studies, with 5 hits from the TargetMol library and 7 hits from the Life Chemicals library showing IC50s of less than 10 μM.

Evans Blue and Salvianolic Acid B are Validated SAMD9 tRNase Inhibitors.

Evans Blue and Salvianolic acid B were the top two hits among the TargetMol library in both the primary screen and the follow-up dose response studies, having IC50s of 1.2 and 3.0 μM, respectively. Evans Blue and Salvianolic acid B were selected, along with Pyridostatin TFA, another TargetMol hit with an IC50 of 7.8 μM, for further studies. It was tested to determine whether these compounds could inhibit SAMD9 tRNase from cleaving its authentic substrate, tRNA-Phe. The cleavage reaction was assessed after two hours of reaction by the gel-based assay. At 2×IC50 concentration from the dose response studies, Evans Blue and Salvianolic acid B blocked, while Pyridostatin TFA partially reduced, the cleavage of yeast tRNA by SAMD9 tRNase.

It was previously shown that SAMD9 tRNase can form a stable complex with tRNA-Phe in an electrophoresis gel mobility shift assay (EMSA). To determine whether the compounds inhibit the cleavage through competitive binding to prevent the complex formation, the abilities of the compounds to disrupt the SAMD9 tRNase:tRNA complex were tested in EMSA. Evans Blue disrupted the complex at a 2:1 molar ratio and completely disrupted it at 20:1, while Salvianolic acid B showed a minor effect at 20:1 and did not completely disrupt it even at 100:1. Pyridostatin caused SAMD9:tRNA aggregate in wells, preventing assessment of its effect on complex formation. Altogether, multiple assays confirm that Evans Blue and Salvianolic acid B effectively inhibit SAMD9 tRNase from cleaving tRNA-Phe.

### DISCUSSION

The discovery of a novel tRNase in SAMD9/9L, along with the revelation that its activity is constitutively activated by pathogenic human mutations, opens a new avenue for developing therapies targeting a spectrum of life-threatening SAMD9/9L disorders. The distinctive fold and substrate specificity of SAMD9/9L tRNase show it as a viable target for selective intervention using small molecules. The present disclosure establishes a facile method for characterizing the enzyme activities and conducted a high-throughput screen that illustrate SAMD9 tRNase as a druggable target. The lead compounds identified from the screen serve as valuable tools for exploring the mechanism of action of small molecule inhibitors and as starting points for the development of drugs with enhanced efficacy and specificity.

Previously, a gel-based assay was employed to elucidate the substrate specificity of SAMD9 tRNase. Notably, the presence of a 2′O-methylated purine base at the wobble position, a distinctive feature unique to eukaryotic tRNA^{Phe}, was found to be essential for substrate recognition. The FRET-based assay developed in the current study faithfully replicates the specificity observed in the gel-based assay, as activity was abolished by either omitting the 2′O-methylation from the substrate or introducing a single mutation in the enzyme's catalytic site. Importantly, the FRET-based assay is rapid and continuous, with high sensitivity and reproducibility, enabling the study of SAMD9 tRNase kinetics and the screening of thousands of small molecules for inhibitors.

After a high-throughput screening of over 32K small molecules, over 40 molecules were found that inhibit the cleavage of the 17-nt by SAMD9 tRNase in a dose-dependent manner. Among the hits showing IC50s of less than 10 μM, Evans Blue and Salvianolic acid B as inhibitors of SAMD9 tRNase cleavage of tRNA-Phe was further validated. Evans Blue was found to be more effective than Salvianolic acid B at disrupting the complex of SAMD9 tRNase with yeast tRNA-Phe, showing that they work by binding at or near the substrate binding pocket of SAMD9 tRNase. Salvianolic acid B is an active ingredient of Salvia miltiorrhiza, which has been widely applied in China for the management of various microcirculation-related disorders. Additional test of the lead compounds as structural studies of SAMD9 tRNase with some of these inhibitors in the future may help the rational design of better inhibitors against SAMD9.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the methods disclosed herein. It is intended that the specification and examples be considered as exemplary only, with a true scope and spirit of the disclosure being indicated by the following claims.

### Sequences

| Name | Sequence | SEQ ID NO |
|---|---|---|
| hATF3-F | CCTCTGCGCTGGAATCAGTC | 1 |
| hATF3-R | TTCTTTCTCGTCGCCTCTTTTT | 2 |
| qPCR-S18-F | CTTCAACCTCCCTCACCATT | 3 |
| qPCR-S18-R | TAGATAGGGCTCAGGCGTTT | 4 |
| tRNA(Phe)-F | GCCGAAATAGCTCAGTTG | 5 |
| tRNA(Phe)-R | CAGGGACCTTTAGATCTTC | 6 |
| tRNA(Leu)-F | GTCAGGATGGCCGAGCGGT | 7 |
| tRNA(Leu)-R | TGTCAGGAGTGGGATTCGA | 8 |
| tRNAPhe-probe-1 | CGAAACCCGGGATCGAACCA | 9 |
| | GGGACCTTTA | |
| tRNAPhe-probe-2 | CTAACGCTCTCCCAACTGAGC | 10 |
| | TATTTCGGC | |
| tRNALeu-probe | TGTCAGAAGTGGGATTCGAA | 11 |
| | CCCACGCCTC | |
| tRNAAla-probe | AGCACGCGCTCTACCACTGA | 12 |
| | GCTACACCCC | |
| tRNAGly-probe | TACCACTGAACCACCAATGC | 13 |
| tRNALys-probe | ACCGACTGAGCTAGCCGGGC | 14 |
| tRNASer-probe | AACCACTCGGCCACCTCGTC | 15 |
| tRNAThr-probe | CCAGTGCTCTAACCACTGAGC | 16 |
| | TATGGAGCC | |
| tRNATyr-probe | TAGTCCTCTGCTCTACCAGCT | 17 |
| | GAACTATTG | |
| tRNA-Phe-Left-side; | GCU CAG UUG GGA GAG CGU | 18 |
| m = 2′-0- | UAG ACUmGAA GAU CUA | |
| methylribose; | AGG UC | |
| Phe-Left-No-O; m = | GCU CAG UUG GGA GAG CGU | 19 |
| 2′-O-methylribose; | UAG ACU GAAGAU CUA AGG | |
| | UC | |
| Phe-anticodon-mut; | GCU CAG UUG GGA GAG CGU | 20 |
| m = 2′-O- | UAG ACU mGUU GAU CUA | |
| methylribose; | AGG UC | |
| Phe-Left-34; m = 2′- | GCU CAG UUG GGA GAG CGU | 21 |
| O-methylribose; | UAG ACUmGAA GAU CUA A | |
| coli-Phe-left-34; m = | GCU CAG UCG GUA GAG CAG | 22 |
| 2′-O-methylribose; | GGC AUUmGAA AAU GCC C | |
| Phe-half-DNA; m = | GCT CAG TTG GGA GAG CGT | 23 |
| 2′-O-methylribose | TAG ACT mGAA GAT CTA | |
| | AGG TC | |
| Phe-17-No-O; | UUA GAC UGA AGA UCU AA | 24 |
| Phe-17-mut1; m = 2′- | UUA GAG AmGU UCU AA | 25 |
| O-methylribose; | | |
| Phe-17-mut2; m = 2′- | GGC UGC UmGA AGA CAG CC | 26 |
| O-methylribose; | | |
| Phe-17-mut3; m = 2′- | UUA GAmC UGA AGA UCU | 27 |
| O-methylribose; | AA | |
| Phe-17-DNA, Phe- | TTA GAC TmGA AGA TCT AA | 28 |
| 17-DNA#2, and Phe- | | |
| 17-DNA#3; m = 2′- | | |
| O-methylribose; | | |
| Phe-17-DNA#4 and | TTA GAC TmGA AGA UCT AA | 29 |
| Phe-17-DNA#5; m = | | |
| 2′-O-methylribose; | | |
| Phe-17-DNA#6; m = | TTA GAC UmGA AGA TCT AA | 30 |
| 2′-O-methylribose; | | |
| Phe-17-WT and Phe- | UUA GAC UmGA AGA UCU | 31 |
| 17-DNA#8; m = 2′- | AA | |
| O-methylribose; | | |
| Phe-17-mut4; m = 2′- | UUA GAC UmCA AGA UCU | 32 |
| O-methylribose; | AA | |
| Phe-17-mut5; m = 2′- | UUA GAC UmAA AGA UCU | 33 |
| O-methylribose; | AA | |
| Phe-17-mut6; m = 2′- | UUA GAC UmUA AGA UCU | 34 |
| O-methylribose; | AA | |
| Phe-17-mut7; m = 2′- | GGC UGC UmGA AGA UCU | 35 |
| O-methylribose; | AA | |
| Phe-17-mut8; m = 2′- | UUA GAA UmGA AGA UCU | 36 |
| O-methylribose; | AA | |
| Phe-17-mut9; m = 2′- | UUA GAU UmGA AGG UCU | 37 |
| O-methylribose; | AA | |
| Phe-17-mut10; m = | UUA GAG UmGA AGA UCU | 38 |
| 2′-O-methylribose; | AA | |
| Phe-17-mut11; m = | UUA GAC AmGA AGA UCU | 39 |
| 2′-O-methylribose; | AA | |
| Phe-17-mut12; m = | UUA GAC CmGA AGA UCU | 40 |
| 2′-O-methylribose; | AA | |
| Phe-17-mut13; m = | UUA GAC GmGA AGA UCU | 41 |
| 2′-O-methylribose; | AA | |
| Phe-17-DNA#7; m = | UUA GAC TmGA AGA UCU AA | 42 |
| 2′-O-methylribose; | | |
| Phe-17-mut13; m = | UUA GAA CUmG AAG UCU | 43 |
| 2′-O-methylribose; | AA | |
| Phe-17-mut14; m = | UUA GAG ACU mGAA UCU | 44 |
| 2′-O-methylribose; | AA | |
| Phe-17-mut15; m = | UUA GAA GAC UmGA UCU | 45 |
| 2′-O-methylribose; | AA | |
| Phe-17-m7-com1; | UUA GAU CUU CAG CC | 46 |
| Phe-17-m7-com2; | UUA GAU AGA ACG CAG CC | 47 |
| Phe-17-mut16; m = | UAG ACU mGAA GAU CUA | 48 |
| 2′-O-methylribose; | | |
| Phe-17-mut17; m = | AGA CUmG AAG AUCU | 49 |
| 2′-O-methylribose; | | |
| Phe-17-mut18; m = | GAC UmGA AGA UC | 50 |
| 2′-O-methylribose; | | |
| Phe-17-mut19; m = | UUA GAC UmGA GAU CUA A | 51 |
| 2′-O-methylribose; | | |
| Phe-17-mut20; m = | UUA GAC UmGG AUC UAA | 52 |
| 2′-O-methylribose; | | |
| Phe-17-mut21; m = | UUA GAC UmGA UCU AA | 53 |
| 2′-O-methylribose; | | |
| Phe-17-mut22; m = | UUA GAU UmGU CUA A | 54 |
| 2′-O-methylribose; | | |
| FTJS1 guide | ATCTGTACCACACCTGGTAG | 55 |
| tRNAPhe (GAA) | TCAGTTGGGAGAGCGTTAGA | 56 |
| | C | |
| tRNALeu (CAA) | TCTAAGGCGCCAGACTCAAG | 57 |
| | T | |
| Ligation adaptor; | AATGATACGGCGACCACCGA | 58 |
| | GATCTACACTCTTTCCCTACA | |
| | CGACGCTCTTCCGATCT GGG | |
| RT Primer; V = | GCATCTCGGTGGTCCAATCTG | 59 |
| guanine/cytosine/ | AACTCCAGTCGTGTGACTGGA | |
| adenine; N = | GTTCAGACGTGTGCTCTTCCG | |
| guanine/cytosine/ | ATCTTTTTTTTTTTTTVN | |
| adenine/thymine | | |
| Forward primer | AATGATACGGCGACCACCGA | 60 |
| | GATCTACACTCTTTCCCTACA | |
| | CGACGCTCT | |
| Library-PCR-R-13 | CAAGCAGAAGACGGCATACG | 61 |
| | AGATTGTTGACT | |
| | GTGACTGGAGTTCAGACGTGT | |
| | GCTCT | |
| Library-PCR-R-14 | CAAGCAGAAGACGGCATACG | 62 |
| | AGATACGGAACT | |
| | GTGACTGGAGTTCAGACGTGT | |
| | GCTCT | |
| Library-PCR-R-15 | CAAGCAGAAGACGGCATACG | 63 |
| | AGATTCTGACAT | |
| | GTGACTGGAGTTCAGACGTGT | |
| | GCTCT | |
| Library-PCR-R-16 | CAAGCAGAAGACGGCATACG | 64 |
| | AGATCGGGACGG | |
| | GTGACTGGAGTTCAGACGTGT | |
| | GCTCT | |
| Library-PCR-R-18 | CAAGCAGAAGACGGCATACG | 65 |
| | AGATGTGCGGAC | |
| | GTGACTGGAGTTCAGACGTGT | |
| | GCTCT | |
| Library-PCR-R-19 | CAAGCAGAAGACGGCATACG | 66 |
| | AGATCGTTTCAC | |
| | GTGACTGGAGTTCAGACGTGT | |
| | GCTCT | |
| Library-PCR-R-20 | CAAGCAGAAGACGGCATACG | 67 |
| | AGATAAGGCCAC | |
| | GTGACTGGAGTTCAGACGTGT | |
| | GCTCT | |
| Library-PCR-R-21 | CAAGCAGAAGACGGCATACG | 68 |
| | AGATTCCGAAAC | |
| | GTGACTGGAGTTCAGACGTGT | |
| | GCTCT | |
| tRNALeu-Forward | ATGCGAATTCCAGACACTGG | 69 |
| | AGAGCCGACCCAGC | |
| tRNALeu-Reverse | ATGCCTGCAGCCCGGTACCTA | 70 |
| | AAAGCCGATTCTT | |
| tRNAPhe-Forward | ATCTAAAGGTCCCTGGTTCGA | 71 |
| | TCCCGGGTTTCGGCACATACT | |
| | TTTCTTTTTCCTCTCCTTAC | |
| tRNAPhe-Reverse | CTTCAGTCTAACGCTCTCCCA | 72 |
| | ACTGAGCTATTTCGGCACACT | |
| | GCATAACAGCCCTGATTT | |
| CRISPR-FTSJ1#1S | CACCGATCTGTACCACACCTG | 73 |
| | GTAG | |
| CRISPR-FTSJ1#1C | AAACCTACCAGGTGTGGTAC | 74 |
| | AGATC | |
| FTSJ1-NheI | CTATCAGCTAGCATGGGACG | 75 |
| | GACGTCAAAGGA | |
| FTSJ1-BamHI | TAATGGATCCTTAAGGTGAAC | 76 |
| | AACTCATTT | |
| EcoRI-FTSJ1-A26P | TGGAATTCCTTATCCAGTTGT | 77 |
| | AGAAGCTTGAAGGGGCTGCG | |
| | AGCA | |
| EcoRI-FTSJ1-K28A | TGGAATTCCTTATCCAATTGT | 78 |
| | AGCAGTGCGAAGGCGCTGCG | |
| | AG | |
| 5′-SacII-hSAMD9 | CTCCGCGGCCGCCCCCTTCAC | 79 |
| | CATGGCAAAGCAACTTAAC | |
| hSAMD9-Hind3 | GGCTTTCTTTGTAAGCTTTGA | 80 |
| | CCAC | |
| h9-E184A-F | AGTCTACAGCCGGCAACAGG | 81 |
| | ACCAGGCAATCT | |
| h9-E184A-R | CTGGTCCTGTTGCCGGCTGTA | 82 |
| | GACTAAAATCC | |
| h9-D192A-F | ATCTCATTGCTCCGATTCATG | 83 |
| | AATTCAAAGCCTTC | |
| h9-D192A-R | GAATTCATGAATCGGAGCAA | 84 |
| | TGAGATTGCCTGGTC | |
| h9-E196A-F | ATCCGATACATGCATTCAAAG | 85 |
| | CCTTCACAAA | |
| h9-E196A-R | AGGCTTTGAATGCATGTATCG | 86 |
| | GATCAATGA | |
| hD9-E218A-F | GAAATTTAGCAATGCGGTTTT | 87 |
| | CCGATTTGCTTCA | |
| hD9-E218A-R | CAAATCGGAAAACCGCATTG | 88 |
| | CTAAATTTCATCTT | |
| hD9-D241A-F | TTTTGGAGTCAAAGCGAAAC | 89 |
| | CCCATGGGAAAATT | |
| hD9-D241A-R | TCCCATGGGGTTTCGCTTTGA | 90 |
| | CTCCAAAATGAAT | |
| hD9-D345A-F | ACTATTTGTGCGAGCGGGGA | 91 |
| | CCAGCTCTAAGGAC | |
| hD9-D345A-R | TAGAGCTGGTCCCCGCTCGCA | 92 |
| | CAAATAGTGAGAA | |
| qATF3-F2 | AGAGGCGACGAGAAAGAAAT | 93 |
| | AA | |
| qATF3-R2 | TCACACTTTCCAGCTTCTCC | 94 |
| qActb-F | CACCATTGGCAATGAGCGGTT | 95 |
| | C | |
| qActb-R | AGGTCTTTGCGGATGTCCACG | 96 |
| | T | |
| hATF3-F | CCTCTGCGCTGGAATCAGTC | 97 |
| hATF3-R | TTCTTTCTCGTCGCCTCTTTTT | 98 |
| qPCR-S18-F | CTTCAACCTCCCTCACCATT | 99 |
| qPCR-S18-R | TAGATAGGGCTCAGGCGTTT | 100 |
| IRNA(Phe)-F | GCCGAAATAGCTCAGTTG | 101 |
| tRNA(Phe)-R | CAGGGACCTTTAGATCTTC | 102 |
| tRNA(Leu)-F | GTCAGGATGGCCGAGCGGT | 103 |
| tRNA(Leu)-R | TGTCAGGAGTGGGATTCGA | 104 |
| tRNAPhe-probe-1 | CGAAACCCGGGATCGAACCA | 105 |
| | GGGACCTTTA | |
| tRNAPhe-probe-2 | CTAACGCTCTCCCAACTGAGC | 106 |
| | TATTTCGGC | |
| TRNALeu-probe | TGTCAGAAGTGGGATTCGAA | 107 |
| | CCCACGCCTC | |
| IRNAAla-probe | AGCACGCGCTCTACCACTGA | 108 |
| | GCTACACCCC | |
| tRNAGly-probe | TACCACTGAACCACCAATGC | 109 |
| tRNALys-probe | ACCGACTGAGCTAGCCGGGC | 110 |
| tRNASer-probe | AACCACTCGGCCACCTCGTC | 111 |
| tRNAThr-probe | CCAGTGCTCTAACCACTGAGC | 112 |
| | TATGGAGCC | |
| tRNATyr-probe | TAGTCCTCTGCTCTACCAGCT | 113 |
| | GAACTATTG | |
| 38WT; m = 2′-O- | GCUCAGUUGGGAGAGCGUUA | 114 |
| methylribose; | GACUmGAAGAUCUAAGGUC | |
| 38m -; m = 2′-O- | GCUCAGUUGGGAGAGCGUUA | 115 |
| methylribose; | GACUGAAGAUCUAAGGUC | |
| 38-mut1; m = 2′-O- | GCUCAGUUGGGAGAGCGUUA | 116 |
| methylribose; | GACUmGUUGAUCUAAGGUC | |
| 34WT; m = 2′-O- | GCUCAGUUGGGAGAGCGUUA | 117 |
| methylribose; | GACUmGAAGAUCUAA | |
| 34-mut1; m = 2′-O- | GCUCAGUCGGUAGAGCAGGG | 118 |
| methylribose; | CAUUmGAAAAUGCCC | |
| 17WT; m = 2′-O- | UUAGACUmGAAGAUCUAA | 119 |
| methylribose; | | |
| 17m -; m = 2′-O- | UUAGACUGAAGA UCUAA | 120 |
| methylribose; | | |
| mut1; m = 2′-O- | UUAGAGAmGUUCUUCUAA | 121 |
| methylribose; | | |
| mut2; m = 2′-O- | GGCUGCUmGAAGACAGCC | 122 |
| methylribose; | | |
| mut3; m = 2′-O- | UUAGAmCUGAAGAUCUAA | 123 |
| methylribose; | | |
| mut4; m = 2′-O- | UUAGACUmCAAGAUCUAA | 124 |
| methylribose; | | |
| mut5; m = 2′-O- | UUAGACUmAAAGAUCUAA | 125 |
| methylribose; | | |
| mut6; m = 2′-O- | UUAGACUmUAAGAUCUAA | 126 |
| methylribose; | | |
| mut7; m = 2′-O- | GGCUGCUmGAAGAUCUAA | 127 |
| methylribose; | | |
| mut8; m = 2′-O- | UUAGAAUmGAAGAUCUAA | 128 |
| methylribose; | | |
| mut9; m = 2′-O- | UUAGAUUmGAAGGUCUAA | 129 |
| methylribose; | | |
| mut10; m = 2′-O- | UUAGAGUmGAAGAUCUAA | 130 |
| methylribose; | | |
| mut11; m = 2′-O- | UUAGACAmGAAGAUCUAA | 131 |
| methylribose; | | |
| mut12; m = 2′-O- | UUAGACCmGAAGAUCUAA | 132 |
| methylribose; | | |
| mut13A; m = 2′-O- | UUAGAACUmGAAGUCUAA | 133 |
| methylribose; | | |
| mut13B; m = 2′-O- | UUAGACGmGAAGAUCUAA | 134 |
| methylribose; | | |
| mut14; m = 2′-O- | UUAGAGACUmGAAUCUAA | 135 |
| methylribose; | | |
| mut15; m = 2′-O- | UUAGAAGACUmGAUCUAA | 136 |
| methylribose; | | |
| mut16; m = 2′-O- | UAGACUmGAAGAUCUA | 137 |
| methylribose; | | |
| mut17; m = 2′-O- | AGACUmGAAGAUCU | 138 |
| methylribose; | | |
| mut18; m = 2′-O- | GACUmGAAGAUC | 139 |
| methylribose; | | |
| mut19; m = 2′-O- | UUAGACUmGAGAUCUAA | 140 |
| methylribose; | | |
| mut20; m = 2′-O- | UUAGACUmGGAUCUAA | 141 |
| methylribose; | | |
| mut21; m = 2′-O- | UUAGACUmGAUCUAA | 142 |
| methylribose; | | |
| mut22; m = 2′-O- | UUAGAUUmGUCUAA | 143 |
| methylribose; | | |
| mut23; m = 2′-O- | UUAGACUmGAUAGAUCUAA | 144 |
| methylribose; | | |
| mut24; m = 2′-O- | UUAGACUmGAUGAGAUCUA | 145 |
| methylribose; | A | |
| 38DNA; m = 2′-O- | GCTCAGTTGGGAGAGCGTTA | 146 |
| methylribose; | GACTmGAAGATCTAAGGTC | |
| 17DNA, DNA#2, and | TTAGACTmGAAGATCTAA | 147 |
| DNA #3; m = 2′-O- | | |
| methylribose; | | |
| DNA#4 and DNA#5; | TTAGACTmGAAGAUCTAA | 148 |
| m = 2′-O- | | |
| methylribose; | | |
| DNA#6; m = 2′-O- | TTAGACUmGAAGATCTAA | 149 |
| methylribose; | | |
| DNA#7; m = 2′-O- | UUAGACTmGAAGAUCUAA | 150 |
| methylribose; | | |
| DNA#8; m = 2′-O- | UUAGACUmGAAGAUCUAA | 151 |
| methylribose; | | |
| 7-8 nucleotide | NNNYNmRNNNNNNN | 152 |
| hairpin; Y refers to | | |
| rC, rU; R refers to | | |
| rA, rG; N refers to | | |
| rA, rG, rU, or rC; m | | |
| refers to 2′-O- | | |
| methylribose | | |
| yeast tRNAPhe; Y = | GCGGAUUUAGCUCAGUUGGG | 153 |
| rC, rU; m = 2′-O- | AGAGCGCCAGAmCUmGAAYA | |
| methylribose | UCUGGAGGUCCUGUGUGUUC | |
| | GAUCCACAGAAUUCGCACCA | |
| human tRNA Phe; Y = | GCCGAAAUAGCUCAGUUGGG | 154 |
| rC, rU; m = 2′-O- | AGAGCGUUAGAmCUmGAAY | |
| methylribose | AUCUAAAGGUCCCUGGUUCG | |
| | AUCCCGGGUUUCGGCACCA | |
| Human tRNA Phe | GCCGAAAUAGCUCAGUUGGG | 155 |
| | AGAGCGUUAGAmCUmGAAG | |
| | AUCUAAAGGUCCCUGGUUCG | |
| | AUCCCGGGUUUCGGCACCA | |
| Phe17-Fret | 6-Fam- | 156 |
| | UUAGACUGmGAAGAUCUAA- | |
| | IABKFQ | |
| Phe17-Fret-noM | 6-Fam- | 157 |
| | UUAGACUGGAAGAUCUAA- | |
| | IABKFQ | |
| Phe17-no-6-Fam | CUmGAAGAUCUAA-IABKFQ | 158 |
| 6-Fam-Phe17 | 6-Fam-UUAGA | 159 |

m = 2′-O-methylribose; r = ribose; V = guanine/cytosine/adenine; N = guanine/cytosine/adenine/thymine; 6-Fam = 6-carboxyfluorescein (FAM); IABKFQ = Iowa Black FQ quencher; no-M = no 2′-O-methylribose; no-6-Fam = no 6-carboxyfluorescein

1. A method of treating a pediatric myelodysplastic syndrome in a subject in need thereof, wherein the method comprises administering a pharmaceutically effective amount of a Sterile Alpha Motif Domain-containing 9 (SAMD9) inhibitor or a Sterile Alpha Motif Domain-containing 9 Like (SAM1D9L) inhibitor.

2. The method of claim 1, wherein the SAMD9 inhibitor or the SAMD9L inhibitor is a synthetic transfer RNA (tRNA).

3. The method of claim 2, wherein the synthetic tRNA is a synthetic phenylalanine tRNA (tRNA^{Phe}).

4. The method of claim 1, wherein the SAMD9 inhibitor or SAMD9L inhibitor decreases an activity of a SAMD9 gain of function (GoF) mutation or an activity of a SAMD9L GoF mutation.

5. The method of claim 1, wherein the SAMD9 GoF mutation or the SAMD9L GoF mutation decreases protein synthesis in the subject.

6. The method of claim 1, wherein the pediatric myelodysplastic syndrome is any one of the following disorders selected from the group consisting of refractory cytopenia of childhood (RCC), refractory anemia with excess blasts (RAEB), and RAEB in transformation (RAEB-t).

7. A method of treating an adult ataxia or an adult pancytopenia in a subject in need thereof, wherein the method comprises administering a pharmaceutically effective amount of a Sterile Alpha Motif Domain-containing 9 (SAMD9) inhibitor or a Sterile Alpha Motif Domain-containing 9 Like (SAMD9L) inhibitor.

8. The method of claim 7, wherein the SAMD9 inhibitor or the SAMD9L inhibitor is a synthetic transfer RNA (tRNA).

9. The method of claim 8, wherein the synthetic tRNA is a synthetic phenylalanine tRNA (tRNA^{Phe}).

10. The method of claim 7, wherein the SAMD9 inhibitor or SAMD9L inhibitor decreases an activity of a SAMD9 gain of function (GoF) mutation or an activity of a SAMD9L GoF mutation.

11. The method of claim 7, wherein the SAMD9 GoF mutation or the SAMD9L GoF mutation decreases protein synthesis in the subject.

12. The method of claim 7, wherein the adult ataxia is any one of the following disorders selected from the group consisting of Friedreich's ataxia, Ataxia-telangiectasia, spinocerebellar ataxias, episodic ataxia, acquired ataxia, idiopathic late-onset cerebellar ataxia (ILOCA), and ataxia with vitamin E deficiency.

13. The method of claim 7, wherein the adult pancytopenia comprises the subject presenting with any one of following blood diseases selected from the group consisting of anemia, leukopenia, and thrombocytopenia.

14. A method of treating a pediatric myelodysplastic syndrome in a subject in need thereof, wherein the method comprises administering a vector comprising a phenylalanine tRNA (tRNA^{Phe}) gene.

15. The method of claim 14, wherein the vector is a DNA plasmid, a bacterial vector, a viral vector, or a nanoparticle.

16. The method of claim 14, wherein the method increases protein synthesis in the subject.

17. The method of claim 14, wherein the pediatric myelodysplastic syndrome is any one of the following disorders selected from the group consisting of refractory cytopenia of childhood (RCC), refractory anemia with excess blasts (RAEB), and RAEB in transformation (RAEB-t).

18. A method of treating an adult ataxia or an adult pancytopenia in a subject in need thereof, wherein the method comprises administering a vector comprising a phenylalanine tRNA (tRNA^{Phe}) gene.

19. The method of claim 18, wherein the vector is a DNA plasmid, a bacterial vector, a viral vector, or a nanoparticle.

20. The method of claim 18, wherein the method increases protein synthesis in the subject.

21.-46. (canceled)
